(19) United States
(12) Patent Application Publication
(10) Pub. No.: US 2024/0139079 A1
(43) Pub. Date: May 2, 2024

(54) HOMOGENEOUS BIOPOLYMER SUSPENSIONS, PROCESSES FOR MAKING SAME AND USES THEREOF

(71) Applicant: 11584022 CANADA INC., Montréal (CA)

(72) Inventor: Thomas DI NARDO, Montréal (CA)

(73) Assignee: 11584022 CANADA INC., Montréal, QC (CA)

(21) Appl. No.: 18/258,914

(22) PCT Filed: Dec. 22, 2021
(86) PCT No.: PCT/IB21/62220

§ 371 Date: Jun. 22, 2023

Related U.S. Application Data
(60) Provisional application No. 63/129,890, filed on Dec. 23, 2020.

(51) Int. Cl. A61K 8/02; A61K 8/60; A61K 8/73; A61Q 17/04; A61Q 19/08

(52) U.S. Cl. CPC A61K 8/027; A61K 8/025; A61K 8/60; A61K 8/731; A61K 8/736; A61Q 17/04; A61Q 19/08

(57) ABSTRACT

Described herein are homogeneous biopolymer suspensions, processes for making same and uses thereof. In embodiments the suspension are aqueous suspension made from natural polymers or biopolymers such as chitin, chitosan and cellulose. Described herein are methods and processes to suspend these biopolymers in polar solutions by submitting the biopolymer(s) and polar solvent(s) to high-shearing conditions such as high mechanical energy. In embodiments the high-shearing conditions and/or high mechanical energy is provided by a ball miller. The compositions and formulations of the invention may find numerous applications particularly in the cosmetic industry if formulated as a paste, an ointment, a cream or a lotion.

### CROSS REFERENCE TO RELATED APPLICATION

The present application relates to U.S. provisional patent application No. 63/129,890 filed on Dec. 23, 2020, the content which is incorporated herein by reference in its entirety.

### FIELD OF THE INVENTION

The invention relates to the field of biopolymers, and more particularly to homogeneous suspensions comprising insoluble or semi-soluble biopolymer(s), processes for making same and uses thereof, particularly in the cosmetic industry.

### BACKGROUND OF THE INVENTION

Natural polymers or biopolymers are polymers that are abundant, natural and, renewable, making it an attractive resource for a commercial product. However most abundant biopolymers such as cellulose and chitin are insoluble, thereby limiting or complicating their use. Providing means to suspend these biopolymers in polar solutions (e.g., aqueous solutions) would thus open new commercial applications for these natural molecules, particularly in the cosmetic industry, which requires constant innovation and is permanently searching for new natural, biocompatible, biodegradable and non-toxic ingredients.

Some methods and processes have been proposed in order to try breaking down biopolymers and/or to try producing homogenous biopolymers suspensions. Such methods and processes are described for example in international PCT publications WO 2020/036872 (e.g., starch) and WO 2020/024053 (e.g., chitin and chitosan), and in patent publications US 2004/0176477 (e.g., chitosan), JP1986149237A (e.g., chitin) and JP1986159430A (e.g., chitin and chitosan). Preparation of chitin nanofibers has been also described in the following scientific publication: Wu et al., BioMacromolecules (2014), dx.doi.org/10.1021/bm501416q; Wang et al., Carbohydrate Polymers (2017), dx.doi.org/10.1016/j.carbpol.2017.09.010; Drobrovol'skaya et al., Natural Polymers (2014), dx.doi.org/10.1134/SO965545X15010022; Zhu et al., Chemistry of Materials (2019), 31, 2078-2087; Ifuku and Saimoto, Nanoscale, 2012, 4, 3308; Lv et al. Food Hydrocolloids (2020), doi.org/10.1016/j.foodhyd.2020.106451. Also, the use ball milling for producing nanoparticles from biopolymers has been described by Rochima et al., (Materials Science and Engineering, 193 (2017) 012043 doi:10.1088/1757-899X/193/1/012043), Wani, T. A. et al. (International Journal of Biological Macromolecules (2020), 154: 166-172), Piras, C. C. et al., Nanoscale Adv. (2019), 1: 937-947, Baheti, V. et al., World Journal of Engineering (2012), 9 (1): 45-50, Lin, H. et al. (Journal of Nano Research (2016), 40: 174-179), Kazemimostaghim, M (Powder Technology (2013), 241: 230-235) and patent publications CN107151276B, CN112500584A, CN103980530A, and CN103316641B. However, these methods and processes suffer from different issues because they generally require the presence of chemicals such as acids and/or bases, because they require other techniques such as sonication or ultrasound, and/or because the resulting products or suspensions are not ideal in terms of viscosity, homogeneity, stability, shape and dimensions of particles and fibers, presence of undesirable chemical compounds, etc.

There is thus a need for suspensions made from abundant insoluble biopolymers that are homogeneous and stable. There is particularly a need for biopolymer compositions comprising biopolymer molecules that have been mechanically processed into a stable homogeneous aqueous suspension. There is a related need for compositions comprising biopolymer fibers having of a greater width and/or greater length than those previously described.

There is also a need for simple and inexpensive methods and process for obtaining such compositions and suspensions. There is particularly a need for methods and process not requiring addition of chemical compounds to avoid the presence of undesirable chemical compounds in the end product.

There is also a need for cosmetic compositions comprising stable and homogeneous suspensions that are free from undesirable chemical compounds and that are made from abundant insoluble or semi-soluble biopolymers.

The present invention addresses these needs and other needs as it will be apparent from the review of the disclosure and description of the features of the invention hereinafter.

### BRIEF SUMMARY OF THE INVENTION

According to one aspect, the invention relates to a biopolymer suspension, comprising a suspension of nano-size insoluble and/or semi-soluble particles (e.g., fibers and/or agglomerated spheres) stably dispersed within a polar solvent.

According to another aspect, the invention relates to a biopolymer composition comprising biopolymer molecules that have been mechanically processed into a stable homogeneous suspension.

According to another aspect, the invention relates to a biopolymer composition comprising a stable homogeneous suspension of an insoluble and/or semi-soluble biopolymer in a polar solvent.

According to another aspect, the invention relates to a biopolymer composition comprising: a stable homogeneous suspension of an insoluble biopolymer in a polar solvent.

According to another aspect, the invention relates to a cosmetic composition comprising a biopolymer composition or a stable homogeneous suspension, as defined herein.

According to another aspect, the invention relates to a mechanical process for obtaining a biopolymer composition, comprising subjecting an insoluble and/or semi-soluble biopolymer to mechanical energy in presence of a polar solvent to obtain a stable homogeneous suspension of said insoluble and/or semi-soluble biopolymer(s).

According to another aspect, the invention relates to a process for obtaining a biopolymer composition, comprising subjecting an insoluble and/or semi-soluble biopolymer to high-shearing conditions in presence of a polar solvent until a change of state is observed and a stable homogeneous suspension of the insoluble and/or semi-soluble biopolymer is obtained.

According to another aspect, the invention relates to the use of a biopolymer suspension or biopolymer composition as defined herein, in the manufacture of a cosmetic composition.

According to another aspect, the invention relates to the use of a biopolymer suspension or biopolymer composition as defined herein, in the manufacture of a seed coating, a surgical implant coating and/or as a food additive.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

In order for the invention to be readily understood, embodiments of the invention are illustrated by way of example in the accompanying figures.

FIG. 1 is an organigram depicting a desired change of state and formulations having decreasing viscosities, in accordance with one embodiment of the present invention.

FIG. 2 is a bar graph showing the results of sizing analysis from SEM of particle size, in accordance with Example 3.

FIG. 3A is a bar graph showing the results of sizing analysis from SEM of fiber width, in accordance with Example 3.

FIG. 3B is a bar graph showing the results of sizing analysis from SEM of fiber length, in accordance with Example 3.

FIGS. 3C to 31 show the powder X-ray diffraction (pXRD) patterns for dry commercial chitin (FIG. 3C) and for samples 3A-F (FIGS. 3D-31, respectively) in accordance with Example 3.

FIG. 4 is a scanning electron microscopy (SEM) micrograph at a 1000× magnification of dried chitin obtained from a chitin suspension, in accordance with Example 2.

FIG. 5 is a scanning electron microscopy (SEM) micrograph at a 1000× magnification of dried chitin obtained from a chitin suspension, in accordance with Example 2.

FIG. 6 is a scanning electron microscopy (SEM) micrograph at a 30 000× magnification of dried chitin obtained from a chitin suspension, in accordance with Example 1.

FIG. 7 is a scanning electron microscopy (SEM) micrograph at a 50 000× magnification of dried chitin obtained from a chitin suspension, in accordance with Example 1.

FIG. 8 is a scanning electron microscopy (SEM) micrograph at a 30 000× magnification of dried chitin obtained from a chitin suspension, in accordance with Example 1.

FIG. 9 is a line graph showing the results of dynamic modulus of suspended chitin at chitin:water ratio of 0.75:20, in accordance with Example 1.

FIG. 10 is a scanning electron microscopy (SEM) micrograph at a 10 000× magnification of a dried chitin obtained from a pretreated chitin suspension, in accordance with Example 2.

FIG. 11 is a line graph showing the results of dynamic modulus of a pretreated chitin suspension at a chitin:water ratio of 1.5:20, in accordance with Example 2.

FIGS. 12A and 12B are pictures of transparent plastic tubes comprising non-milled chitin (FIG. 12A) and suspended milled chitin (FIG. 12B), in accordance with Example 4.

FIGS. 13A and 13B are pictures of transparent plastic tubes comprising non-milled chitosan (FIG. 13A) and suspended milled chitosan (FIG. 1bB), in accordance with Example 4.

FIGS. 14A and 14B are pictures of transparent plastic tubes comprising non-milled alpha-cellulose (FIG. 14A) and suspended milled alpha-cellulose (FIG. 14B), in accordance with Example 4.

FIGS. 15A and 15B are pictures of transparent plastic tubes comprising non-milled cellulose fibers (FIG. 15A) and suspended milled cellulose fibers (FIG. 15B), in accordance with Example 4.

FIGS. 16A and 16B are pictures of transparent plastic tubes comprising non-milled microcrystalline cellulose (FIG. 16A) and suspended milled microcrystalline cellulose (FIG. 16B), in accordance with Example 4.

FIGS. 17A and 17B are pictures of transparent plastic tubes comprising non-milled collagen (FIG. 17A) and suspended milled collagen (FIG. 17B), in accordance with Example 4.

FIGS. 18A and 18B are pictures of transparent plastic tubes comprising non-milled silk (FIG. 18A) and suspended milled silk (FIG. 18B), in accordance with Example 4.

FIGS. 19A and 19B are pictures of transparent plastic tubes comprising suspended milled mixtures of chitin and chitosan, in accordance with Example 5.

FIG. 20 is a picture of a transparent plastic tube comprising a suspended milled mixture of chitin+beeswax, in accordance with Example 6.

FIGS. 21A, 21B, and 21C, are pictures of transparent plastic tubes comprising suspended milled mixtures of chitin and vegetable oil, in accordance with Example 6.

FIG. 22 is a picture of a transparent plastic tube comprising a suspended milled mixture of chitin and soybean oil, in accordance with Example 6.

FIG. 23 is a picture of a transparent plastic tube comprising a suspended milled mixture of chitin with two solvents (glycerol+water), in accordance with Example 7.

FIG. 24A is a line graph of FTIR of silk powder, dried post-suspension, in accordance with Example 8.

FIG. 24B is a line graph of FTIR of cellulose powder, dried post-suspension, in accordance with Example 8.

FIG. 24C is a line graph of FTIR of collagen powder, dried post-suspension, in accordance with Example 8.

FIG. 24D is a line graph of FTIR of alginic acid powder, dried post-suspension, in accordance with Example 8.

FIG. 24E is a line graph of FTIR of chitin powder, dried post-suspension, in accordance with Example 8.

FIG. 24F is a line graph of FTIR of chitosan powder, dried post-suspension, in accordance with Example 8.

FIG. 25A is a line graph of SSNMR of silk, in accordance with Example 8.

FIG. 25B is a line graph of SSNMR of cellulose, in accordance with Example 8.

FIG. 25C is a line graph of SSNMR of collagen, in accordance with Example 8.

FIG. 25D is a line graph of SSNMR of alginic acid, in accordance with Example 8.

FIG. 25E is a line graph of SSNMR of chitin, in accordance with Example 8.

FIG. 25F is a line graph of SSNMR of chitosan, in accordance with Example 8.

FIG. 26A is a line graph of the PXRD of silk powder, dried post-suspension, in accordance with Example 8.

FIG. 26B is a line graph of the PXRD of cellulose powder, dried post-suspension, in accordance with Example 8.

FIG. 26C is a line graph of the PXRD of collagen powder, dried post-suspension, in accordance with Example 8.

FIG. 26D is a line graph of the PXRD of alginic acid powder, dried post-suspension, in accordance with Example 8.

FIG. 26E is a line graph of the PXRD of chitin powder, dried post-suspension, in accordance with Example 8.

FIG. 26F is a line graph of the PXRD of chitosan powder, dried post-suspension, in accordance with Example 8.

FIGS. 27A-27F are line graphs of transmittance of suspensions, in accordance with Example 10, for silk (FIG. 27A), for cellulose (FIG. 27B), for collagen (FIG. 27C), for alginic acid (FIG. 27D), for chitin (FIG. 27E) and chitosan (FIG. 27F).

FIGS. 28A-28C are pictures of SEM imaging, for alginic acid at 15 mins (FIG. 28A and FIG. 28B), 1 hour (FIG. 28C), and 3 hours (FIG. 28D and FIG. 28E), in accordance with Example 11.

FIGS. 29A-29D are pictures of SEM imaging, for cellulose at 15 mins (FIG. 29A), 1 hour (FIG. 29B), and 3 hours (FIG. 29C and FIG. 29D), in accordance with Example 11.

FIGS. 30A-30D are pictures of SEM imaging, for chitin at 15 mins (FIG. 30A), 1 hour (FIG. 30B), and 3 hours (FIG. 30C and FIG. 30D), in accordance with Example 11.

FIGS. 31A-31D are pictures of SEM imaging, for chitosan at 15 mins (FIG. 31A and FIG. 31B), 1 hour (FIG. 31C), and 3 hours (FIG. 31D), in accordance with Example 11.

FIGS. 32A-32G are pictures of SEM imaging, for silk at 15 mins (FIG. 32A and FIG. 32B), 1 hour (FIGS. 32C and 32D), and 3 hours (FIG. 32E, FIG. 32F and FIG. 32G), in accordance with Example 11.

FIG. 33 is a line graph showing rheology polymer sweeps of polymer suspensions and blends thereof, in accordance with Example 13.

FIG. 34 is a line graph showing viscosity of chitin that has been pre-milled or not, in accordance with Example 14.

FIG. 35A depicts the chemical structure of N-Acetyl Glucosamine.

FIG. 35B depicts an estimation from ChemDraw™ of the ¹H NMR spectrum for N-Acetyl Glucosamine ¹H NMR.

FIGS. 36A and 36B depict ¹HNMR spectra for two separate chitin suspensions, in accordance with Example 15.

FIG. 36C depicts ¹HNMR spectra for an N-Acetyl Glucosamine Standard (bottom) compared with chitin suspension #2 (top), in accordance with Example 15.

FIGS. 37A and 37B are pictures showing ginseng powder in water non-milled (FIG. 37A) and ginseng milled and suspended (FIG. 37B), in accordance with Example 19.

FIG. 38A is a graph showing particle size distribution of chitin milled at 200 RPM, in accordance with Example 23.

FIG. 38B is a picture showing SEM imaging of chitin milled at 200 RPM for 180 minutes, in accordance with Example 23.

FIG. 39A is a graph showing particle size distribution of chitin milled at 400 RPM, in accordance with Example 23.

FIG. 39B is a picture showing SEM imaging chitin milled at 400 RPM for 180 minutes, in accordance with Example 23.

FIG. 40 is a graph showing particle size distribution of chitin no mill, in accordance with Example 23.

FIG. 41 is a graph showing particle size distribution of chitin standard mill, in accordance with Example 23.

FIG. 42A is a graph showing particle size distribution of chitosan milled at 200 RPM, in accordance with Example 23.

FIG. 42B is a picture showing SEM imaging of chitosan milled at 200 RPM for 180 minutes, in accordance with Example 23.

FIG. 43A is a graph showing particle size distribution of chitosan milled at 400 RPM, in accordance with Example 23.

FIG. 43B is a picture showing SEM imaging of chitosan milled at 400 RPM for 180 minutes, in accordance with Example 23.

FIG. 44 is a graph showing particle size distribution of chitosan no mill, in accordance with Example 23.

FIG. 45 is a graph showing particle size distribution of chitosan standard mill, in accordance with Example 23.

FIG. 46A is a graph showing particle size distribution of cellulose milled at 200 RPM, in accordance with Example 23.

FIG. 46B is a picture showing SEM imaging of cellulose milled at 200 RPM for 180 minutes, in accordance with Example 23.

FIG. 47A is a graph showing particle size distribution of cellulose milled at 400 RPM, in accordance with Example 23.

FIG. 47B is a picture showing SEM imaging of cellulose milled at 400 RPM for 180 minutes, in accordance with Example 23.

FIG. 48 is a graph showing particle size distribution of cellulose no mill, in accordance with Example 23.

FIG. 49 is a graph showing particle size distribution of cellulose standard mill, in accordance with Example 23.

FIG. 50 is a line graph showing viscosity of a chitin suspension with an emulsifier, a preservative and/or oil, in accordance with Example 27.

FIG. 51 is a line graph showing viscosity of a cellulose suspension with an emulsifier, a preservative and/or oil, in accordance with Example 27.

Further details of the invention and its advantages will be apparent from the detailed description included below.

### DETAILED DESCRIPTION OF EMBODIMENTS

In the following description of the embodiments, references to the accompanying figures are illustrations of examples by which the invention may be practiced. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

The invention generally relates to the preparation of stable homogeneous suspensions of insoluble and/or semi-soluble biopolymers in a polar solvent. Associated aspects concern biopolymer compositions comprising such suspensions, uses thereof for commercial applications such as in cosmetic products, and processes for obtaining the suspensions.

The present inventors have found means to suspend insoluble and/or semi-soluble biopolymers in polar solvents, thereby providing useful commercial applications for these abundant natural molecules. The essence of the invention relies on subjecting the insoluble and/or semi-soluble biopolymers to mechanical energy in presence of a polar solvent under conditions resulting in a stable homogeneous suspension of the insoluble and/or semi-soluble biopolymer. In embodiments the mechanical energy comprises high-shearing conditions and the viscosity of the suspension can be altered by varying these high-shearing and input material conditions.

### Biopolymer Compositions

One aspect of the invention concerns biopolymer compositions comprising biopolymer molecules (e.g., insoluble and/or semi-soluble) that have been mechanically processed into a stable homogeneous aqueous suspension.

A related aspect concerns biopolymer compositions comprising a stable homogeneous suspension of an insoluble and/or semi-soluble biopolymer in a polar solvent.

As used herein, the term “homogeneous suspension” or “homogeneous composition”, refers to a suspension or composition which appears to be uniform, as determined by visual inspection. However, the suspension or composition would still qualify as “homogenous” even if it comprises particles of different dimensions or sizes (e.g., a range of particles sizes or length) or if it comprises particles of different shapes (e.g., spherical particles, fibers, etc.). Preferably, homogeneous suspensions or homogeneous compositions in accordance with the present invention are also “stable”, i.e., upon visual inspection, there is no or limited phase separation of their constituents for hours, days or weeks. Stable homogeneous suspensions or homogeneous compositions may display be some solvent separation (e.g., depending on the biopolymer, solvent content, elapsed time after milling, etc.) but typically they do not display precipitation of solids from the suspension.

As used herein, the term “biopolymer” refers to natural polymers produced by the cells of living organisms. Biopolymers consist of monomeric units that are covalently bonded to form larger molecules. The present invention encompasses polypeptides, polysaccharides and polynucleotides biopolymers that are insoluble or semi-soluble in water as defined hereinafter. Other examples of biopolymers include natural rubbers (polymers of isoprene), suberin and lignin (complex polyphenolic polymers), cutin and cutan (complex polymers of long-chain fatty acids) and melanin. In embodiments the biopolymers used as starting materials and obtained in the suspensions are substantially pure, i.e., they consist of only purified natural polymers. Preferably, the biopolymers are substantially free from chemical residues and any of such chemical residue is absent or present in undetectable or trace amounts (see definition of “substantially free from chemical residues” hereinafter).

As used herein, the term “insoluble biopolymer” refers to a biopolymer that is “insoluble” in a polar solvent (particularly water) and this term encompasses equivalent terms such as “non-water-soluble”, or “not soluble in water”, or “water-insoluble” or “indissoluble”. Insolubility can typically be observed by a separation, i.e., two separate phases in an aqueous mixture, for instance biopolymer deposits/sediments at a bottom or floating at the top of the aqueous mixture. In accordance with the present invention, examples of insoluble biopolymers include, but are not limited to, chitin, chitosan, cellulose, hemicellulose, lignin, amylose, actin, fibrin, collagen, silk, fibroin, keratin, wool, alginic acid and mixtures thereof.

As used herein, the term “semi-soluble biopolymer” refers to a biopolymer that may be solubilized in a polar solvent such as water, but under certain conditions (e.g., molecular weight, heat, addition of chemicals such as acids, alcohols, surfactants, etc.). In accordance with the present invention, examples of semi-soluble biopolymers include, but are not limited to gelatin, pectin, starch, amylopectin, agarose, hyaluronic acid, RNA, DNA, xanthan gum, latex, polymannans, suberin, cutin, cutan, and mixtures thereof.

As used herein, the terms “insoluble biopolymer” and the term “semi-soluble biopolymer” are meant to contrast with the term “soluble biopolymer”, the latter referring to a biopolymer that can be solubilized in a polar solvent such as water. A biopolymer is considered soluble when there is no observed phase separation between the biopolymer and the solvent in a mixture consisting essentially of the biopolymer and the solvent. The present invention is directed to the use of insoluble and/or semi-soluble biopolymers and is not meant to encompass biopolymer suspensions made from soluble biopolymers. Examples of known soluble biopolymers (or source of biopolymers) that are excluded from the scope of the present invention include those failing the phase separation test as defined hereinbelow.

Those skilled in the art appreciate the fact that, for certain compounds, the molecular weight can have an influence on solubility in a particular solvent, e.g., higher molecular weight biopolymers are typically less soluble than smaller molecular weight biopolymers. Therefore, in accordance with the present invention, the same biopolymer can fill into different categories (i.e., “insoluble”, “semi-soluble” and “soluble”), its molecular weight typically determining its behaviour in a solvent (i.e., insoluble, semi-soluble, or soluble).

In accordance with the present invention, it is envisionable to have a “phase separation test” to identify in advance biopolymers that are most suitable for obtaining a biopolymer suspension in accordance with the present invention, wherein a polymer which phase separates would be a good candidate for obtaining a biopolymer suspension in accordance with the present invention. In one embodiment the phase separation test may comprise combining the biopolymer in a powder form with the desired solvent at standard temperature and pressure (STP), where the polymer either dissolves fully in the solvent (soluble) or partially dissolves or swells (semi-soluble) or does not dissolve and fully phase separates (insoluble).

The good candidates for obtaining a biopolymer suspension in accordance with the present invention would be the biopolymers that would pass the phase separation test, i.e., compounds that phase separates when mixed with a solvent. For instance, it has been found that typically pectin and gelatin would fail the phase separation test, whereas lignin would pass sometimes, depending on its source. Examples of biopolymers that would fail the test, i.e., biopolymers that do not separate because they are already soluble include, but are not limited to, sodium hyaluronate, sodium alginate, hydrolyzed collagen, carrageenan, guar gum, and xantham gum. Without wishing to be bound to any theory, as indicated hereinbefore, solubility likely depends on the molecular weight of the biopolymer. Those skilled in the art will be able to identify insoluble and semi-soluble biopolymers that are useful in accordance with the recent invention in view of the present definitions, the present detailed description and/or the numerous examples provided hereinafter in the Exemplification section.

As mentioned above, the present invention encompasses mixtures of two, three, four, five or more insoluble biopolymers including, but not limited to, chitin+chitosan, chitin+cellulose, chitin+collagen, chitin+silk, chitosan+silk, chitosan+cellulose, chitosan+collagen, cellulose+collagen, cellulose+silk, collagen+silk, etc. The present invention also encompasses mixtures of two, three, four, five or more semi-soluble biopolymers including, but not limited to agarose+DNA, xanthan gum+starch, latex+alginate, xantham gum+DNA, guar gum+cutan, etc. It may also be envisioned to mix together two, three, four, five or more insoluble and semi-soluble biopolymers including but not limited to chitin+agarose, chitosan+agarose, chitin+gelatin, chitin+xanthan gum, chitosan+xanthan gum, chitin+sodium hyaluronate, chitosan+sodium hyaluronate, cellulose+sodium hyaluronate, chitin+agarose, chitosan+agarose, cellulose+agarose,

In accordance with the present invention, suitable solvents include those that are able to form hydrogen bonds between the solvent and the biopolymer as greater hydrogen bonding ability will increase suspension stability. Suitable solvents include polar protic solvents, polar aprotic solvents and mixture thereof.

In embodiments the solvent is a polar solvent which allows to suspend the biopolymers molecules into a stable homogeneous suspension. In embodiments the solvent is a polar solvent which allows to suspend the biopolymers molecules into a stable colloidal homogeneous suspension. The polar solvent may be a polar protic solvent or a polar aprotic solvent. The polar solvent may be an aqueous solvent. The present invention encompasses the use of more than one solvent in the same or in different categories.

Envisioned examples of polar protic solvents that could be used include, but are not limited to, water, ethanol, propanol, methanol, glycerol, isopropanol, acetic acid, nitromethane, n-butanol, formic acid, isopropanol, 1-propanol, ethanol, methanol, acetic acid, water, glycerol, ethylene glycol, diethylene glycol, pentanol, cyclohexanol, hexanol, heptanol, octanol, 2-amino ethanol, benzyl alcohol, aniline, diethylamine and mixtures thereof. In embodiments the polar protic solvent is water (e.g., distilled water).

Envisioned examples of polar aprotic solvents that could be used include, but are not limited to, acetone, ethyl acetate, acetonitrile, dimethyl formamide, dimethyl sulfoxide, hexamethylphosphoramide, dichloromethane, dimethylpropyleneurea, hexamethylphosphoric triamide, tetrahydrofuran, dimethylsulfoxide, acetyl acetone, ethyl acetoacetate, benzonitrile, pyridine, diglyme, ethyl benzoate, methoxybenzene, tetrahydrofuran, pentanone, methyl acetate, ether, and mixtures thereof.

Envisioned examples of aqueous solvents that could be used include, but are not limited to, water, ethanol, propanol, methanol and glycerol, etc. and mixtures thereof. In embodiments the solvent is water (e.g., distilled water). Furthermore, numerous examples of potentially useful polar protic solvents and dipolar aprotic solvents are provided hereinafter.

Those skilled in the art will be able to identity the solvent(s) that fits best for a particular use. For instance, some solvents may be less preferable to others because they may not be safe for human applications. Likewise, ethanol and propanol may for instance be useful for a hand sanitizer but not for a face cream while solvents such as ethylacetate, acetonitrile, dimethyl formamide, dimethyl sulfoxide may be useful for industrial applications but not necessarily for human or cosmetic applications.

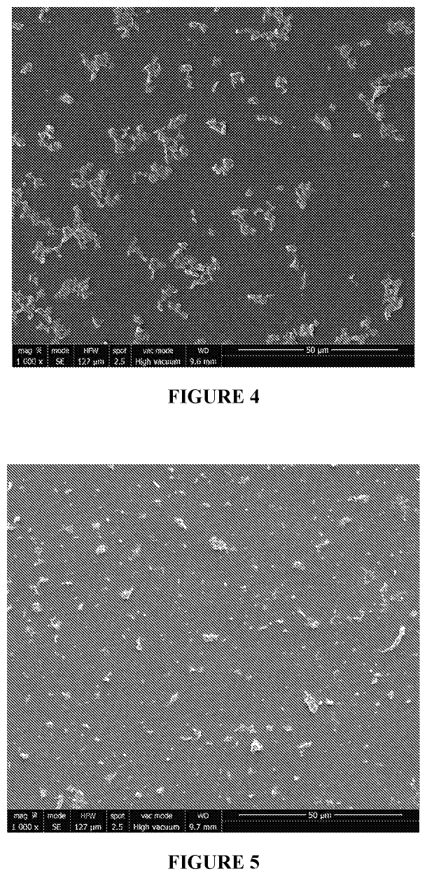

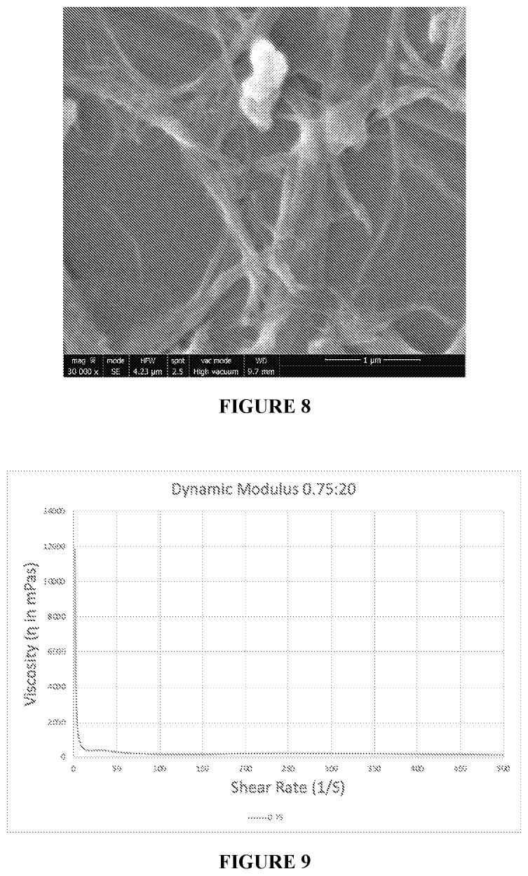

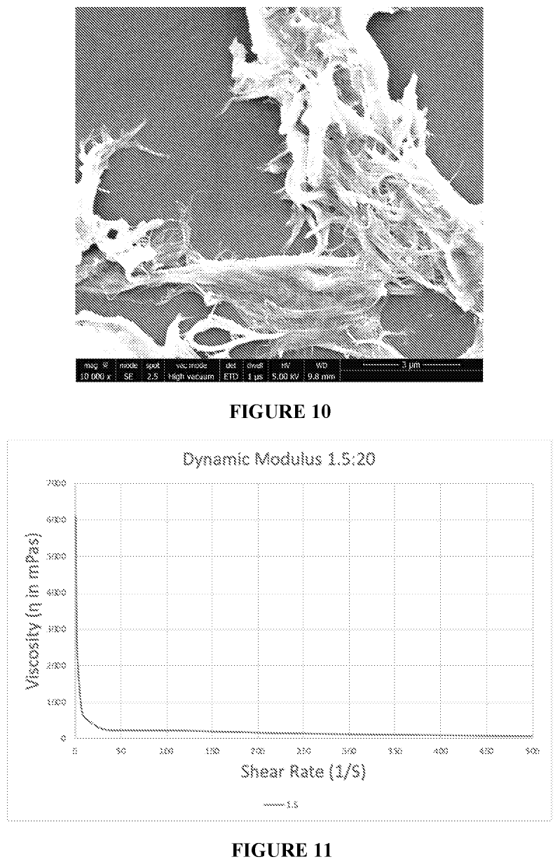

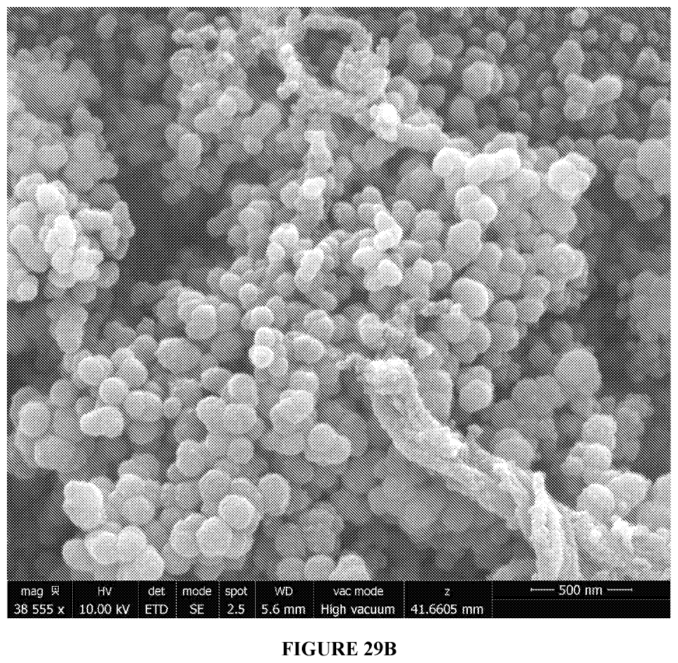

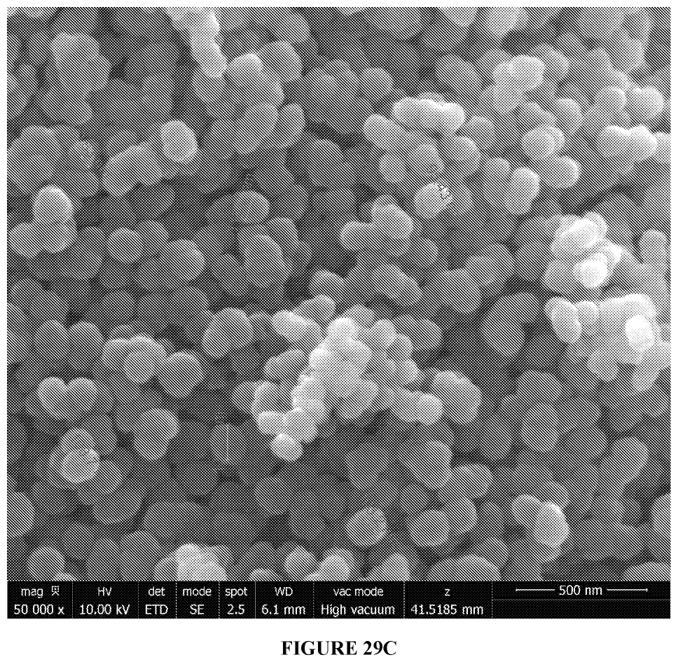

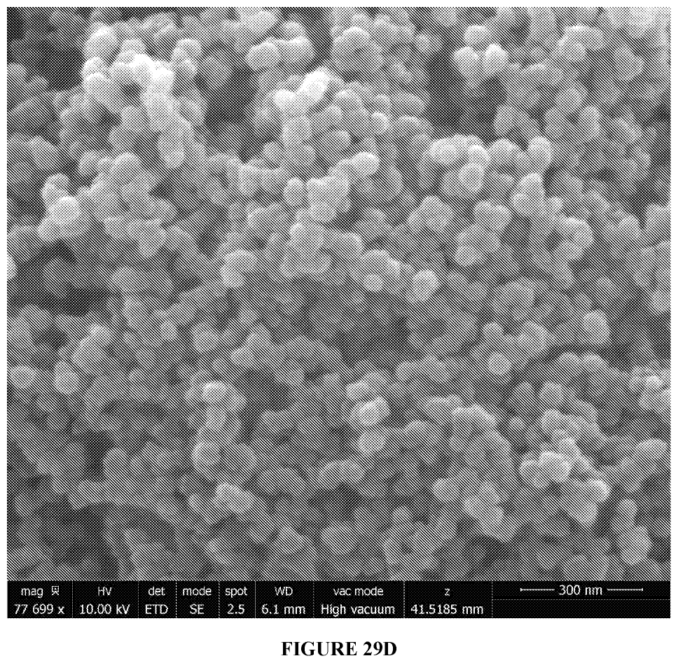

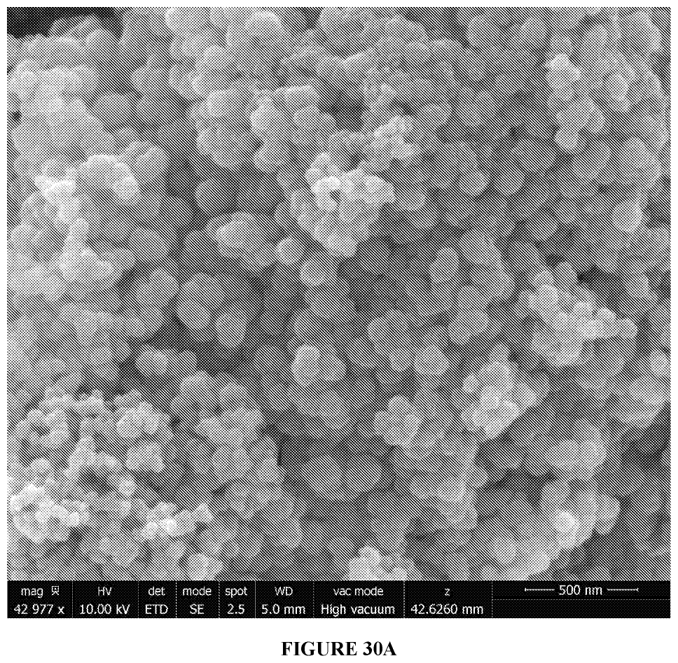

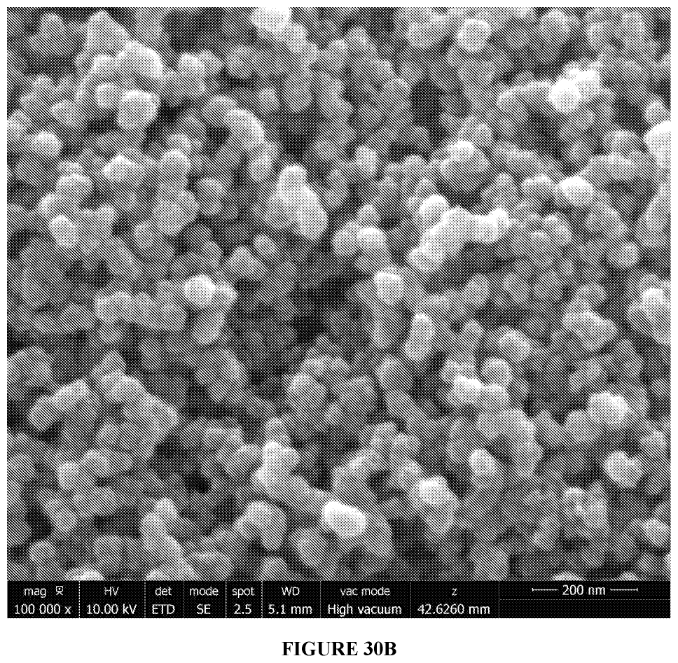

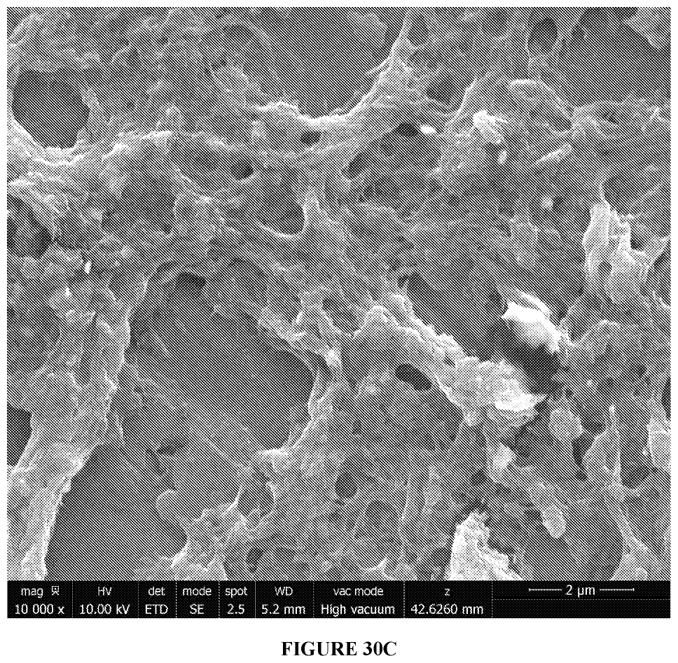

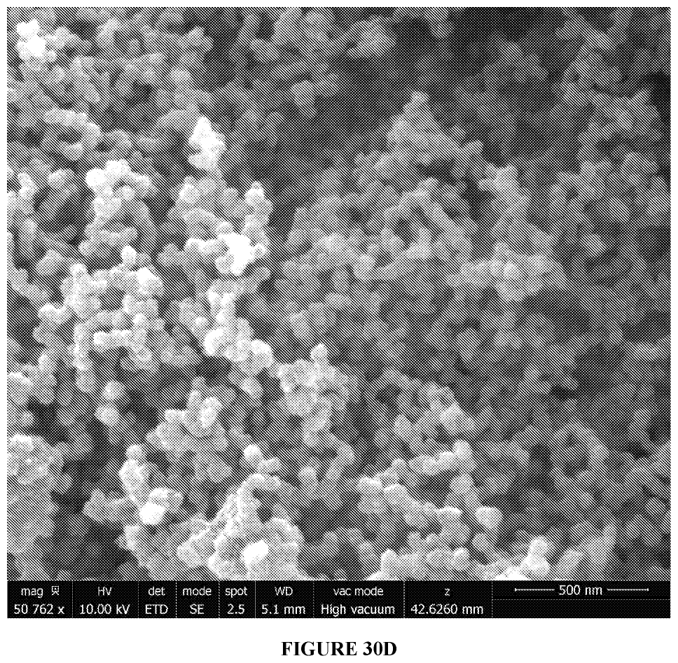

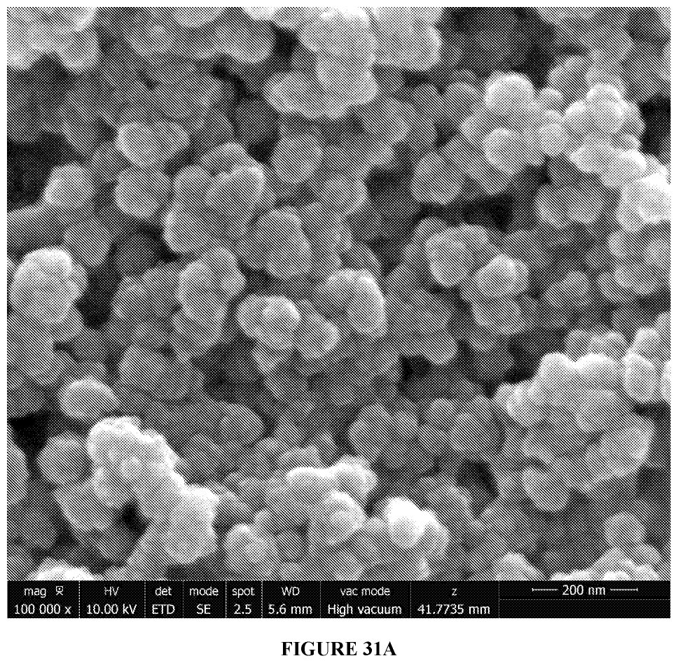

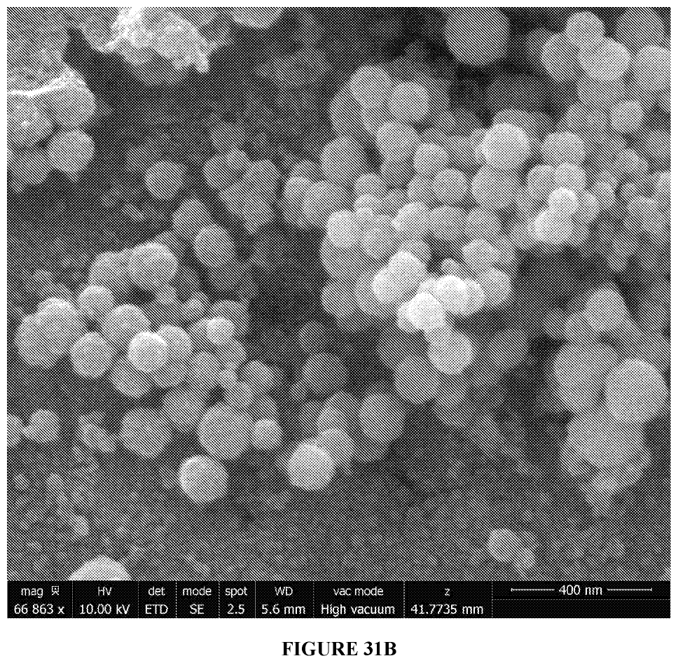

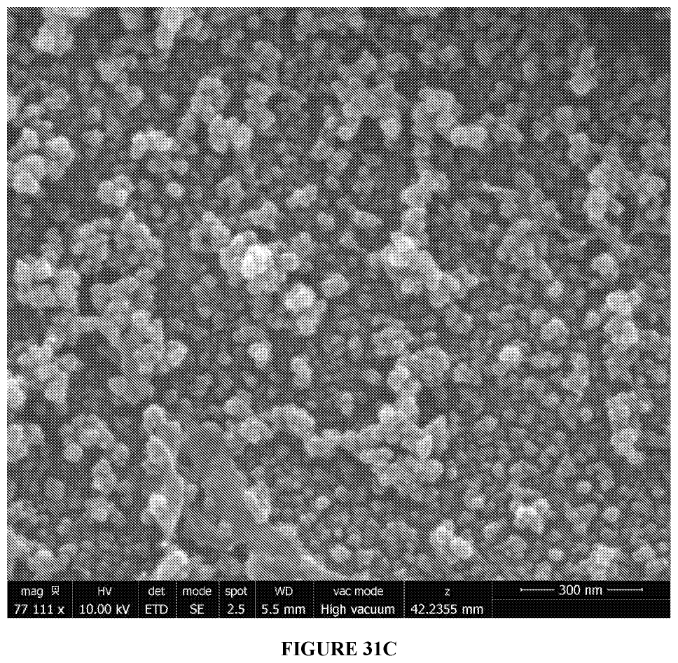

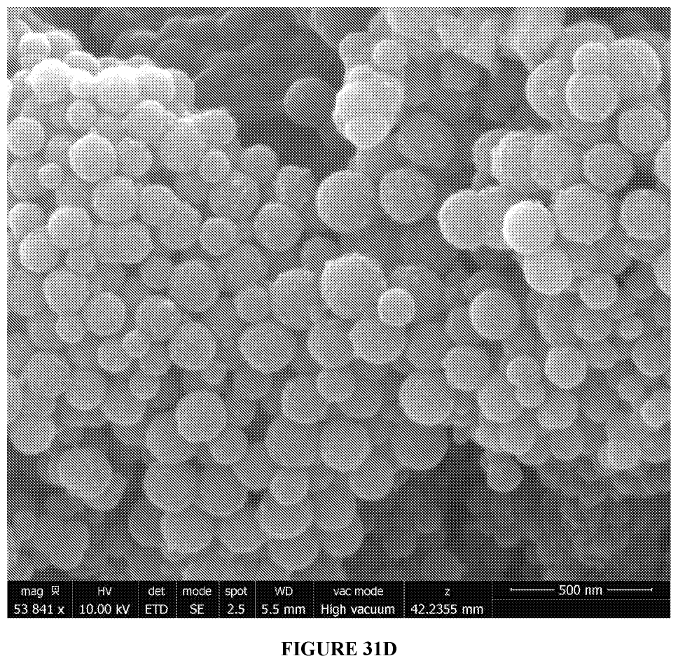

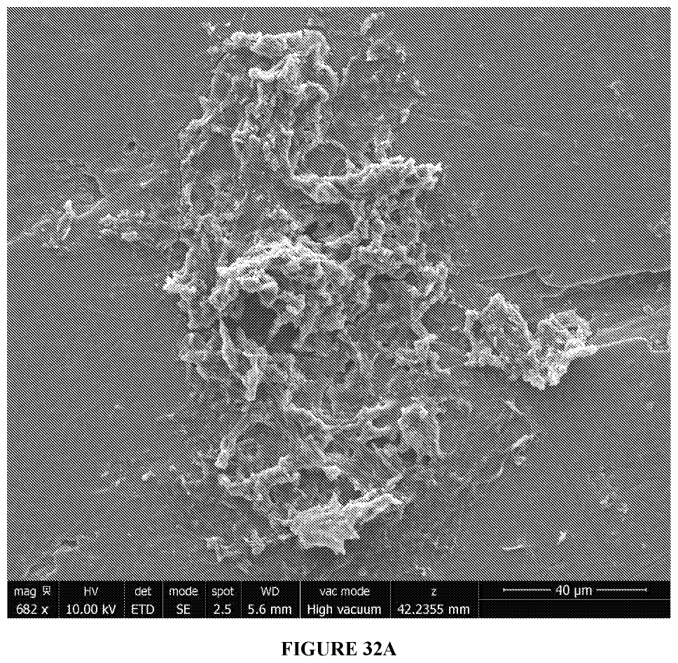

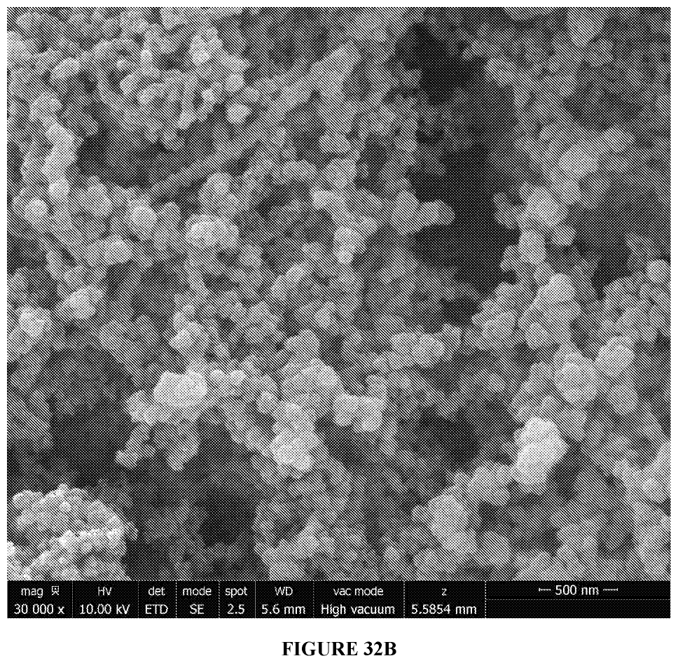

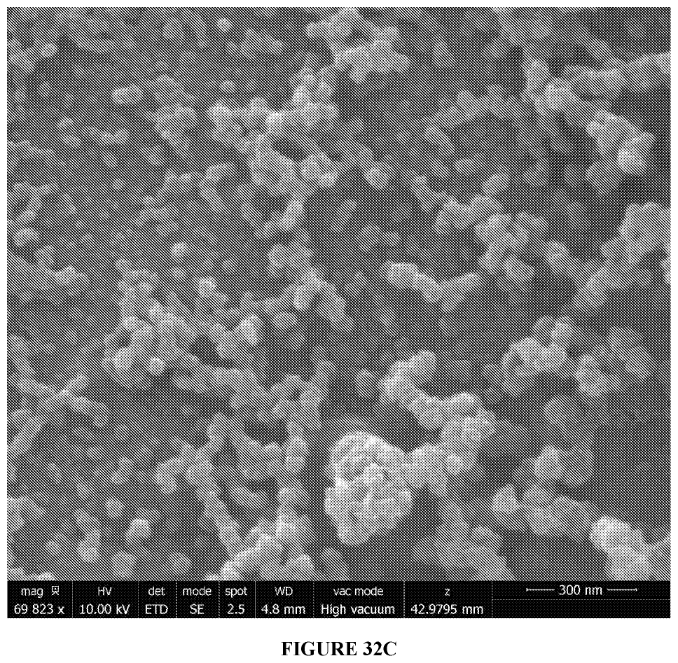

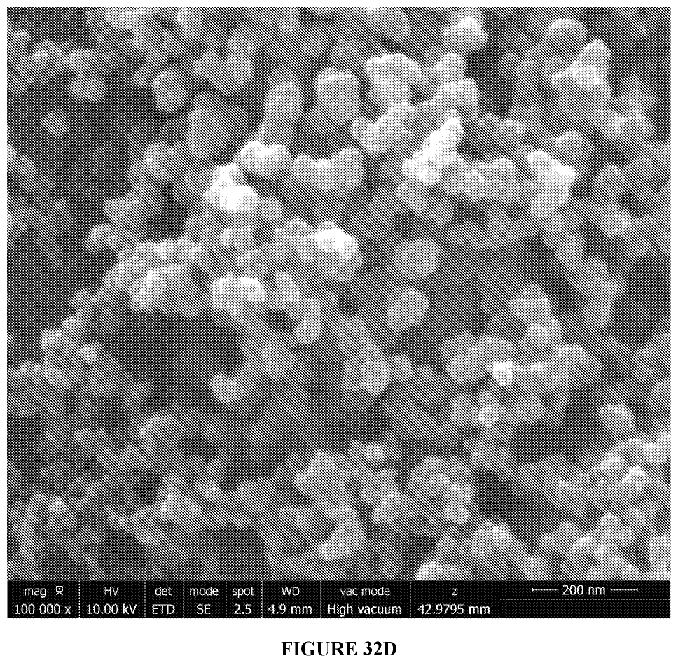

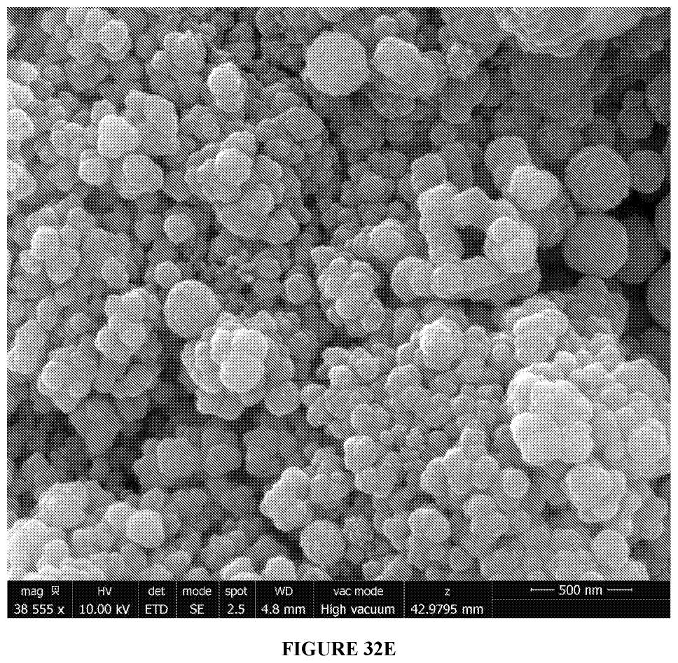

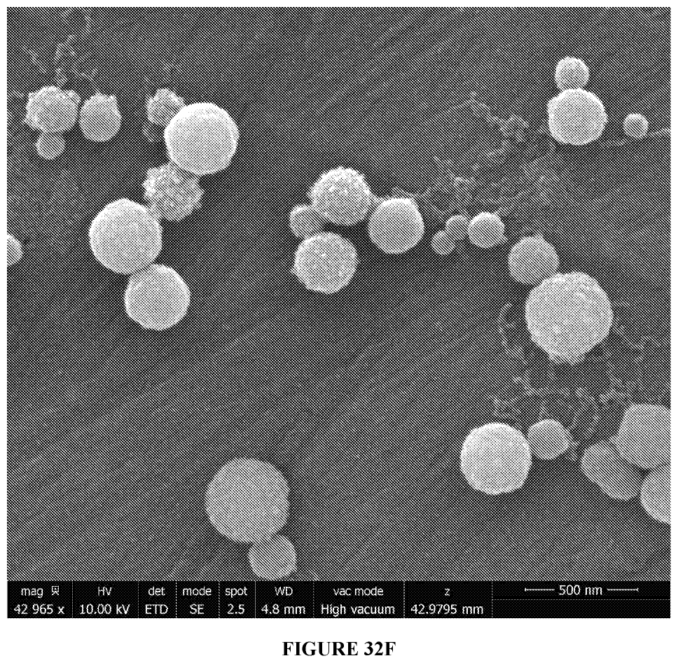

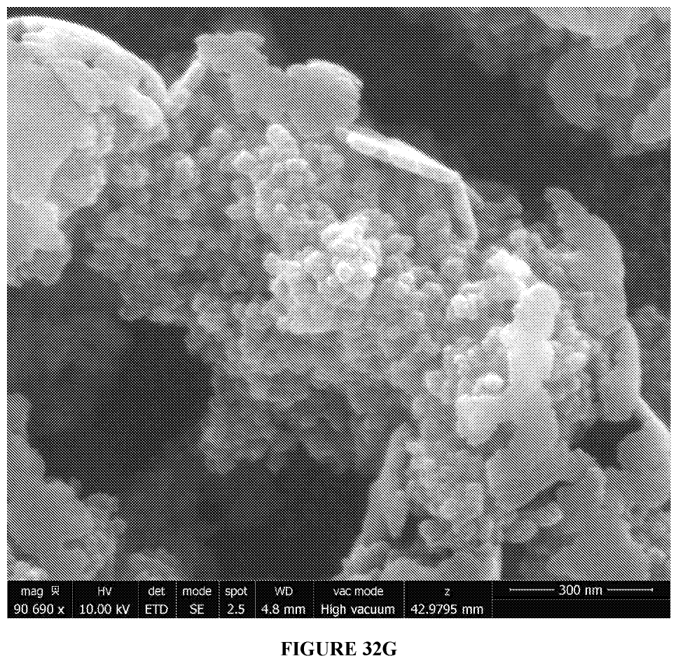

The nano-size insoluble and/or semi-soluble particles that are present in biopolymer suspensions in accordance with the present invention may be shaped like fibers and/or like agglomerated spheres or agglomerated bodies. In embodiments, the biopolymer suspension comprises particles having a shape similar to the particles illustrated in any of FIGS. 4-8, 10, 28A-28E, 29A-29D, 30A-30D, 31A-31D, 32A-32G, 38B, 39B, 42B, 43B, 46B, and 47B.

Without being bound by any theory, it is hypothesized that greater shearing force (e.g., more milling power, longer milling duration, etc.) will cause the original biopolymer molecules (usually found in fiber form) become smaller biopolymer molecules (e.g., spherical bodies), with fibers being typically larger than spherical bodies. For instance, there might be a first defibrillation step where the fibers separate from one another for becoming thinner and shorter. Further in the process the fibers get much shorter and can aggregate into spheres, especially upon drying. As such, it is conceivable in accordance with the present invention to obtain biopolymer suspensions comprising particles of a desired shape or desired size by controlling the shearing force being applied to the original biopolymer molecules (e.g., milling speed, milling power, number and/or size of the ball). Additional factors or conditions that may affect the shape and size of the particles in the final suspension include, but are not limited to, the source or identity of the starting material(s), initial particle size, the quantity of materials, the solvent(s), the additive(s), the numbers and/or size of balls in the case of a milling machine, etc. Accordingly, in embodiments the present invention encompasses modifying or controlling one or more of these parameters and/or shearing conditions (e.g., milling conditions) in order to change the shape and/or size of the particles in the biopolymer suspension. It is also envisionable to do cryo-SEM for imaging the compositions or suspensions in a pseudo wet (frozen) state in order to obtain information on how the particles look in suspension, and compared these images with images in a dried form to further visualized and optimize accordingly the preparation of particles (e.g., fibers, spheres) having desired characteristics (e.g., size, diameter, length, etc.).

In embodiments, the homogeneous suspension is a colloidal homogeneous suspension. In embodiments, the colloidal homogeneous suspension comprises colloids having a range from about 1 nm to about 1 μm.

In embodiments, the stable homogeneous suspension comprises biopolymer fibers. In embodiments the stable homogeneous suspension comprises biopolymer fibers having of a width of about 7 nm to about 5 μm, or about 10 nm to about 5 μm, or about 20 nm to about 5 μm, or about 25 nm to about 5 μm, or about 30 nm to about 5 μm, or about 35 nm to about 5 μm, or about 35 nm to about 3 μm.

In embodiments the stable homogeneous suspension comprises biopolymer fibers having of a width of at least 10 nm, or at least 20 nm, or at least 30 nm, or at least 40 nm, or at least 50 nm, or at least 75 nm, or at least 100 nm, or at least 250 nm, or at least 500 nm, or at least 750 nm, or at least 1 μm, or at least 2 μm, or at least 3 μm, or at least 4 μm, or at least 5 μm, or at least 10 μm or wider.

In embodiments the stable homogeneous suspension comprises biopolymer fibers having of a length of about 50 nm to about 10 μm, or about 100 nm to about 10 μm, or about 500 nm to about 10 μm, or about 750 nm to about 10 μm, or about 800 nm to about 10 μm, or about 900 nm to about 5 μm, or about 1 μm to about 10 μm, or about 1 μm to about 5 μm, or about 1 μm to about 3 μm.

In embodiments the stable homogeneous suspension comprises biopolymer fibers having of a length of at least 50 nm, or at least 100 nm, or at least 250 nm or at least 500 nm, or at least 750 nm, or at least 800 nm, or at least about 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or longer.

In embodiments the stable homogeneous suspension comprises biopolymer fibers having both: (i) a width greater than 20 nm (e.g., at least 25 nm, or at least 40 nm, or at least 50 nm) and a length greater than 50 nm (e.g., at least 100 nm, or at least 500 nm, or at least 1 μm, or at least 2 μm); or (ii) a width greater than 32 nm (e.g., at least 35 nm, or at least 40 nm, or least 50 nm) and a length of than 50 nm (e.g., at least 100 nm, or at least 500 nm, or at least 1 μm, or at least 2 μm); or (iii) a width greater than 20 nm (e.g., at least 25 nm, or at least 40 nm, or least 50 nm) and a length of than 500 nm (e.g., at least 600 nm, or at least 750 nm, or at least 1 μm, or at least 2 μm); or (iv) a width greater than 30 nm (e.g., at least 35 nm, or at least 40 nm, or least 50 nm) and a length of than 800 nm (e.g., at least 900 nm, or at least 1 μm, or at least 2 μm); or (v) a width greater than 8 nm (e.g., at least 10 nm, at least 25 nm, or at least 35 nm, or at least 40 nm, or least 50 nm) and a length of than 340 nm (e.g., at least 350 nm, or at least 500 nm, at least 750 nm, or at least 900 nm, or at least 1 μm, or at least 2 μm); or (vi) a width greater than 11 nm (e.g., at least 15 nm, at least 25 nm, or at least 35 nm, or at least 40 nm, or least 50 nm) and a length of than 166 nm (e.g., at least 200 nm, or at least 350 nm, or at least 500 nm, at least 750 nm, or at least 900 nm, or at least 1 μm, or at least 2 μm); or (viii) a width greater than 32 nm (e.g., at least 35 nm, or at least 40 nm, or least 50 nm) and a length greater than 800 nm (e.g., at least 900 nm, or at least 1 μm, or at least 2 μm, or at least 3 μm, or at least 4 μm, or at least 5 μm).

In embodiments, the stable homogeneous suspension comprises biopolymer fibers wherein the average width and average length of the fibers in the suspension are as defined hereinabove, e.g. an average width greater than 20 nm (e.g., at least 25 nm, or at least 40 nm, or at least 50 nm) and an average length greater than 50 nm (e.g., at least 60 nm, at least 75 nm, or at least 100 nm, or at least 500 nm, at least 750 nm, or at least 1 μm, or at least 2 μm, or at least 3 μm, or at least 4 μm, or at least 5 μm).

In embodiments the stable homogeneous suspension comprises biopolymer fibers having both a crystalline region and an amorphous region. In embodiments the stable homogeneous suspension comprises biopolymer fibers having a globular shape. In embodiments the stable homogeneous suspension is comprised of mainly, or only, of suspended biopolymer nanofibrils.

Those skilled in the art are aware that particle size measurements may vary according to the measurement method and the state of the particles (e.g., particles in a wet state are larger than the same particles in a dry state). Typically, the particles will be in a wet or suspended stage when measured by dynamic light scattering (DLS) and in a dry stage when measured by scanning electron microscopy (SEM).

In embodiments the biopolymer suspension or composition in accordance with the present invention comprises spherical particles and agglomerates and the range of particle sizes, as measured by dynamic light scattering (DLS), is as defined in Table 3 hereinafter.

In embodiments the biopolymer suspension or composition comprises agglomerated spheres of alginic acid having an average size of about 40 nm to about 80 nm, or about 45 nm to about 75 nm, as measured by scanning electron microscopy (SEM). In emblements, the stable homogeneous suspension comprises agglomerated spheres of alginic acid having a median size of about 30 nm to about 70 nm or about 35 nm to about 65 nm, average size of about 40 nm to about 80 nm, or about 45 nm to about 75 nm, as measured by scanning electron microscopy (SEM).

In embodiments the biopolymer suspension or composition comprises agglomerated spheres of cellulose having an average size of about 50 nm to about 80 nm, or about 55 nm to about 75 nm, average size of about 40 nm to about 80 nm, or about 45 nm to about 75 nm, as measured by scanning electron microscopy (SEM). In embodiments the stable homogeneous suspension comprises agglomerated spheres of cellulose having a median size of about 35 nm to about 75 nm or about 40 nm to about 65, average size of about 40 nm to about 80 nm, or about 45 nm to about 75 nm, as measured by scanning electron microscopy (SEM).

In embodiments the biopolymer suspension or composition comprises agglomerated spheres of chitin having an average size of about 45 nm to about 85 nm, or about 50 nm to about 80 nm. In embodiments the stable homogeneous suspension comprises agglomerated spheres of cellulose having a median size of about 45 nm to about 80 nm or about 50 nm to about 75 nm, as measured by scanning electron microscopy (SEM).

In embodiments the biopolymer suspension or composition comprises agglomerated spheres of chitosan having an average size of about 75 nm to about 120 nm, or about 80 nm to about 115 nm, or about 85 nm to about 110 nm, as measured by scanning electron microscopy (SEM). In embodiments the stable homogeneous suspension comprises agglomerated spheres of chitosan having a median size of about 70 nm to about 100 nm or about 75 nm to about 95 nm, as measured by scanning electron microscopy (SEM).

In embodiments the biopolymer suspension or composition comprises agglomerated spheres of silk having an average size of about 40 nm to about 165 nm, or about 45 nm to about 160 nm, as measured by scanning electron microscopy (SEM). In embodiments the stable homogeneous suspension comprises agglomerated spheres of silk having a median size of about 40 nm to about 150 nm or about 45 nm to about 140, as measured by scanning electron microscopy (SEM).

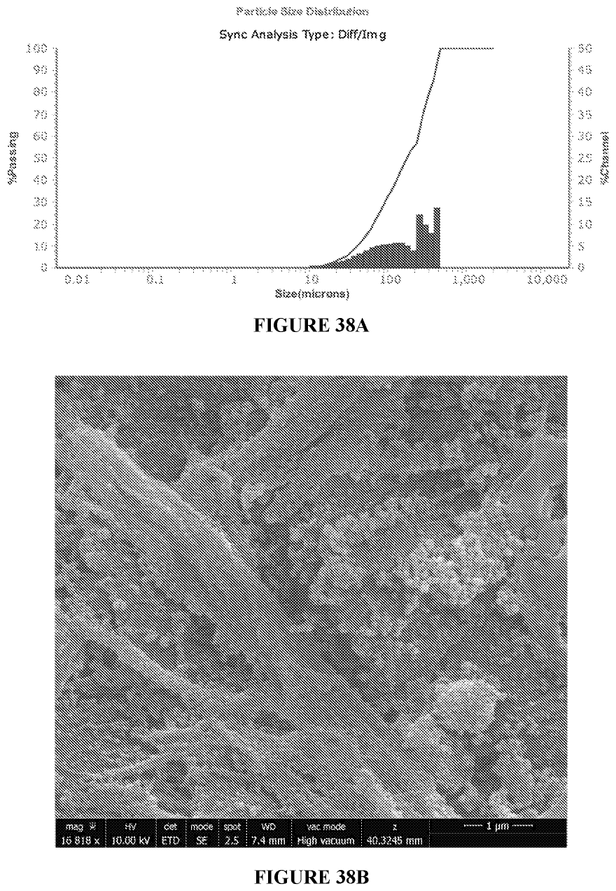

In embodiments the biopolymer suspension or composition in accordance with the present invention comprises particles of one or more of alginic acid, cellulose, chitin, chitosan and silk, wherein the range of particle sizes, as measured by SEM is as defined in Table 4 hereinafter (e.g., Example 11), or as defined in any of Tables 30-44 hereinafter (e.g., Example 23), or as depicted in any one of FIGS. 38A, 39A, 40, 41, 42A, 43A, 44, 45, 46A, 47A, 48 and 49 (e.g., Example 23).

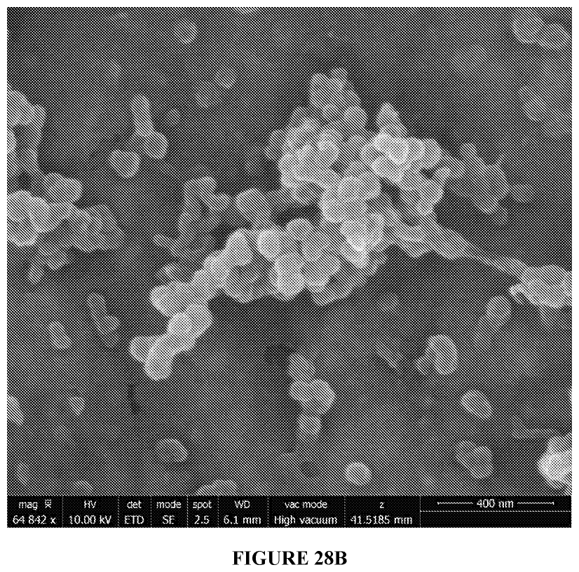

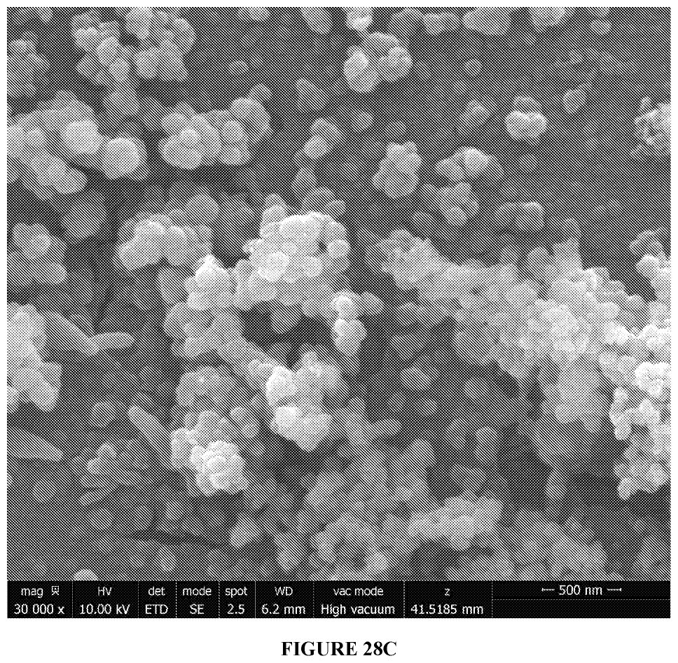

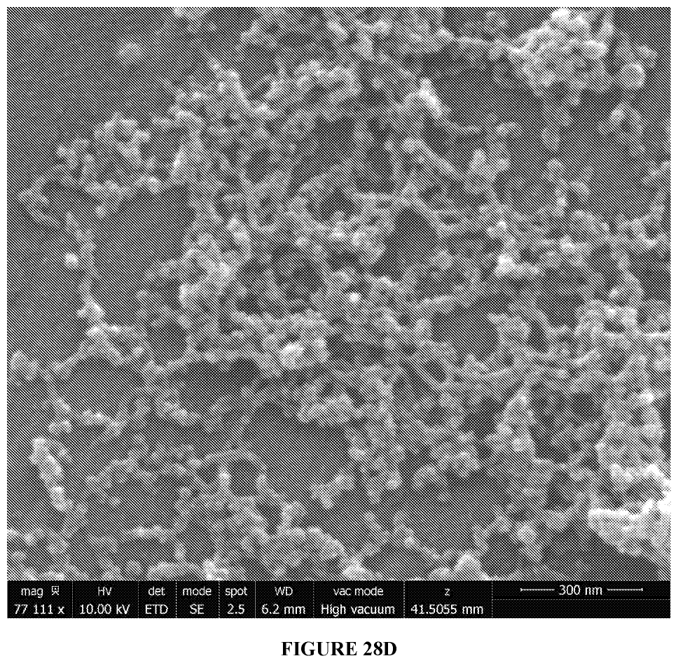

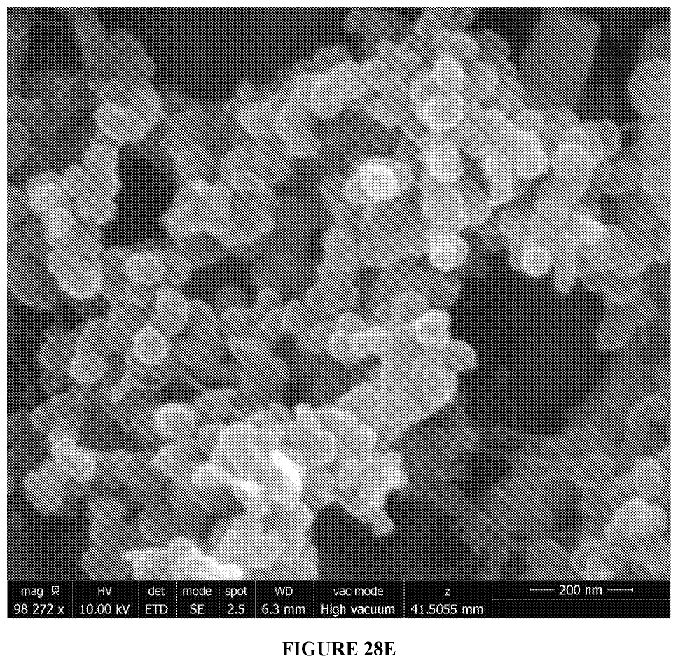

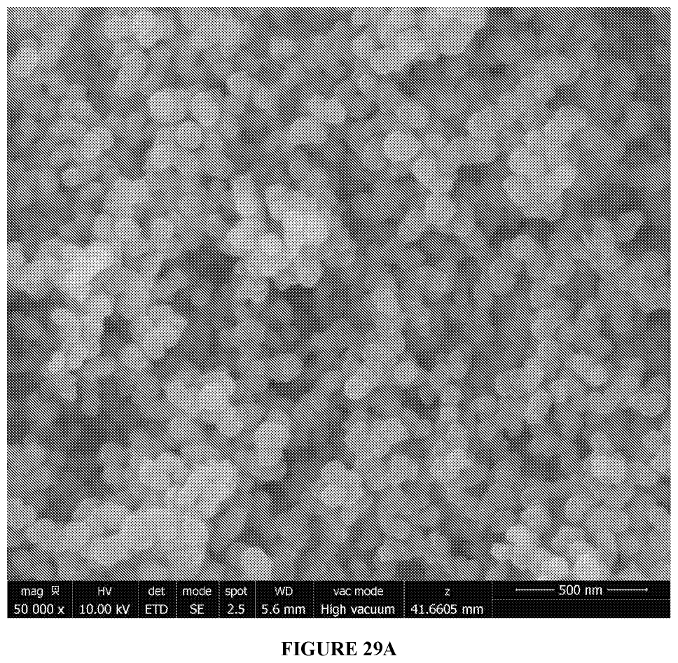

In embodiments, the biopolymer suspension or composition is characterized by visual properties like those depicted in the SEM images shown in any one of FIGS. 28A to 32G (e.g., Example 11) or in any one of FIGS. 38B, 39B, 42B, 43B, 46B and 47B (e.g., Example 23).

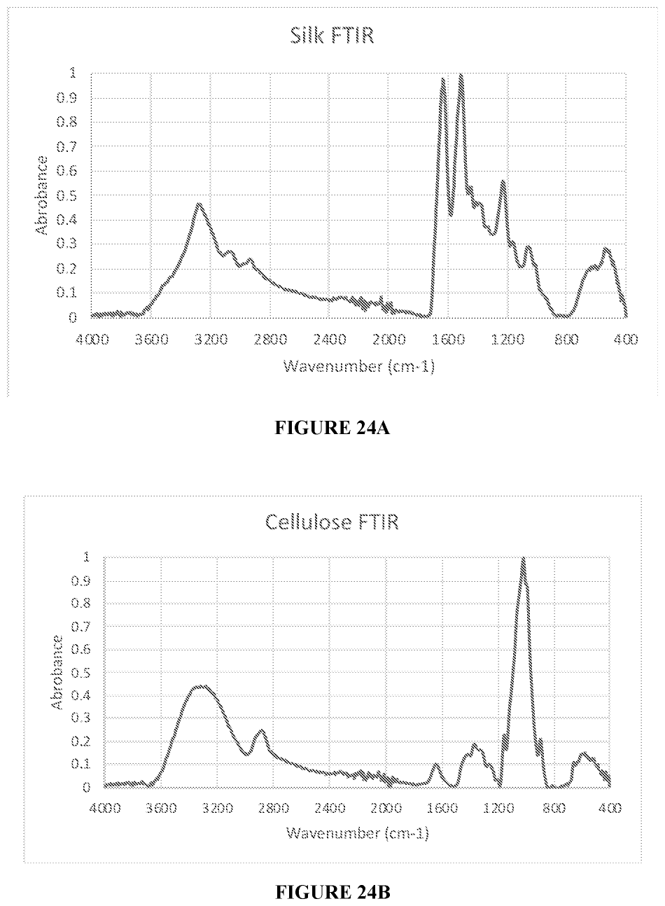

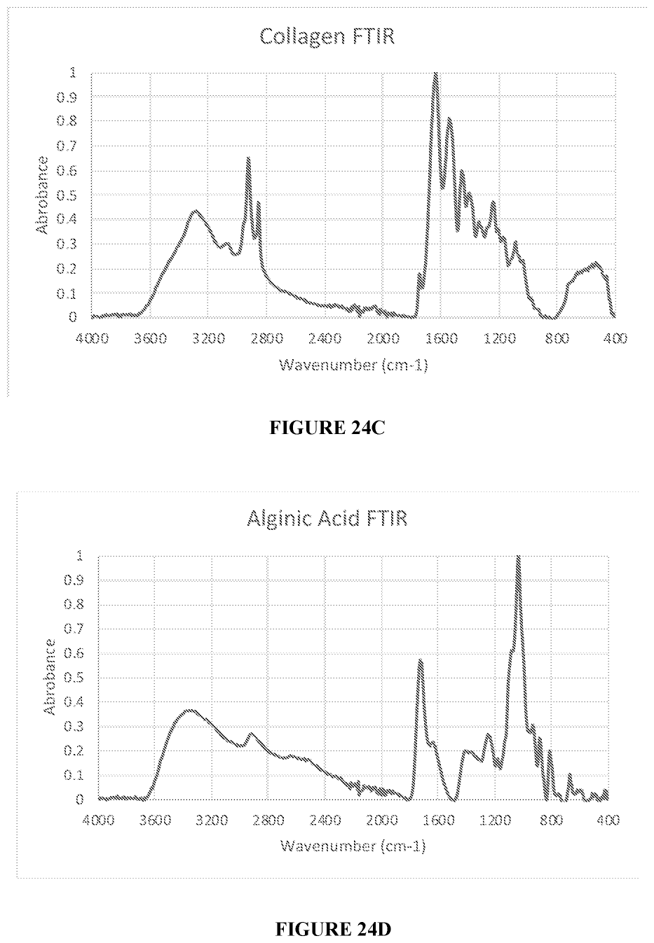

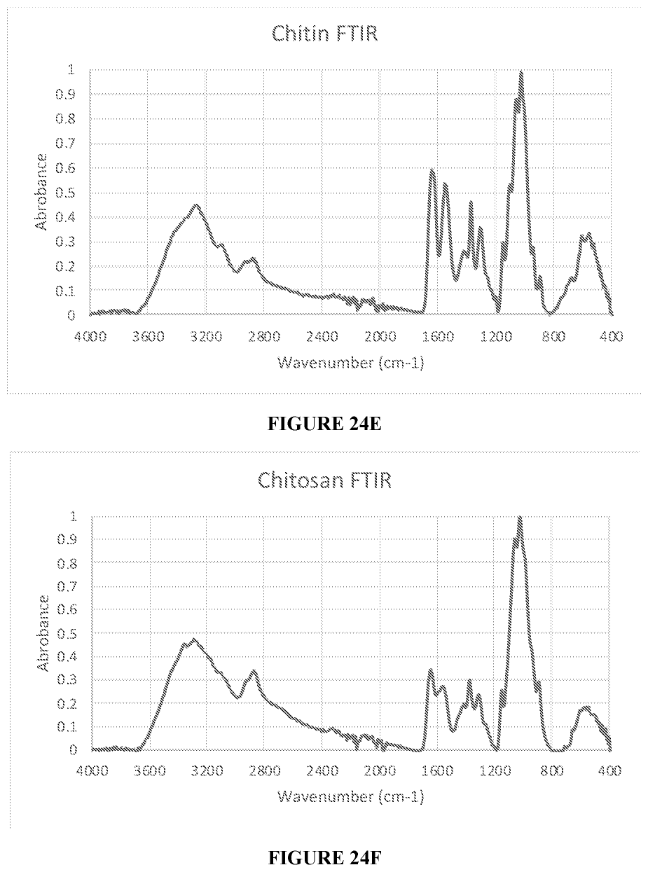

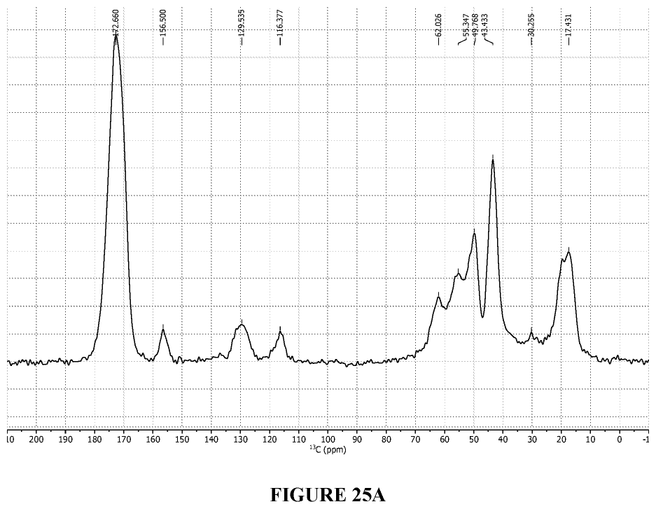

In embodiments, the biopolymer suspension or composition is characterized by a Fourier Transform Infrared Spectroscopy (FTIR) spectrum as depicted in any one of FIGS. 24A to 24F (e.g., Example 8).

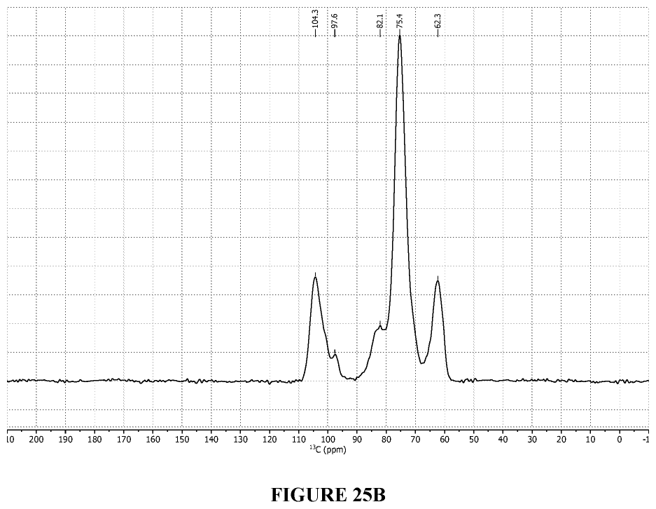

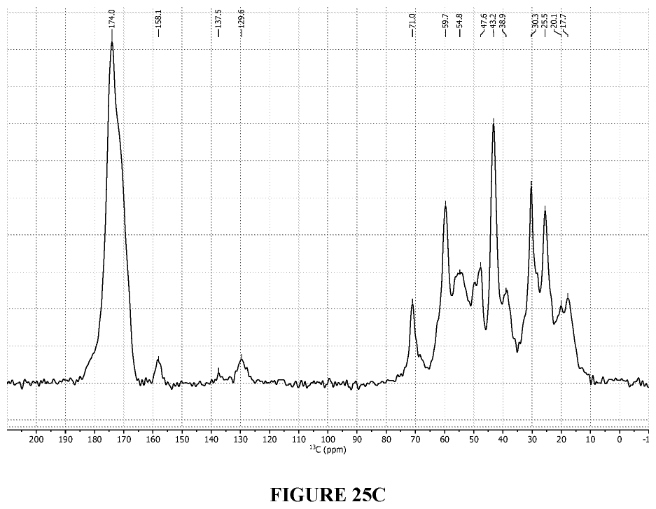

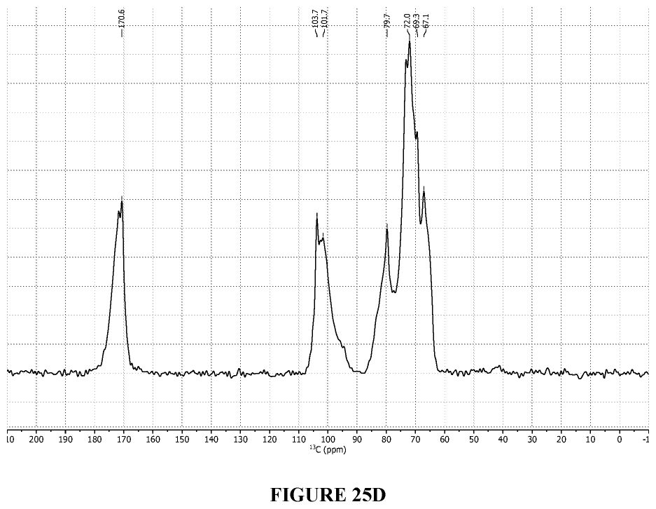

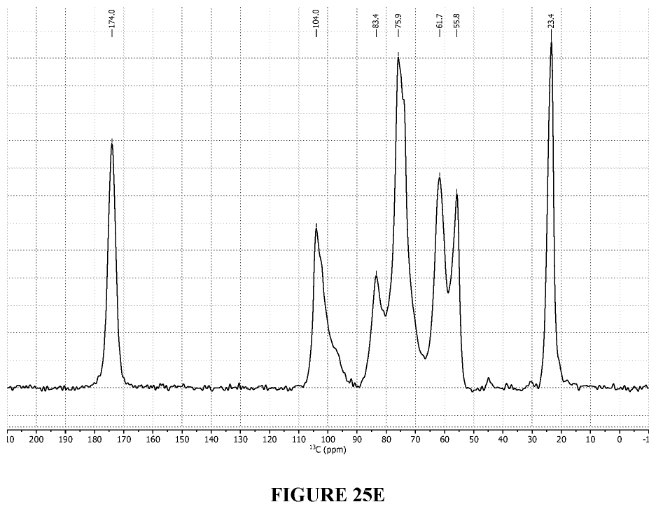

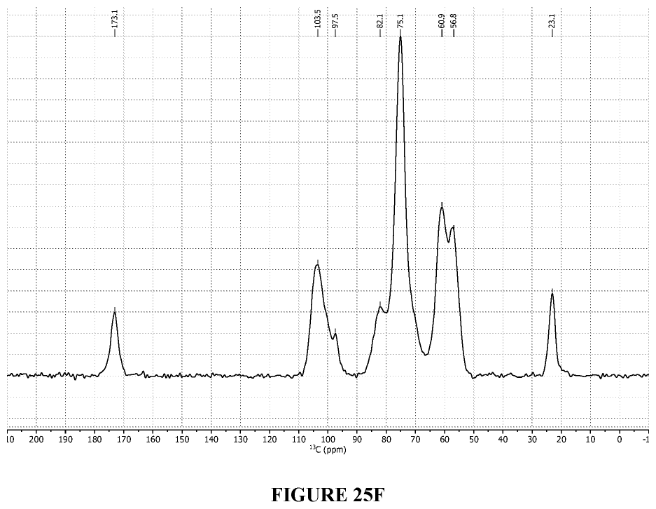

In embodiments, the biopolymer suspension or composition is characterized by Solid-State Nuclear Magnetic Resonance characterization (SSNMR)_as depicted in any one of FIGS. 25A to 25F (e.g., Example 8).

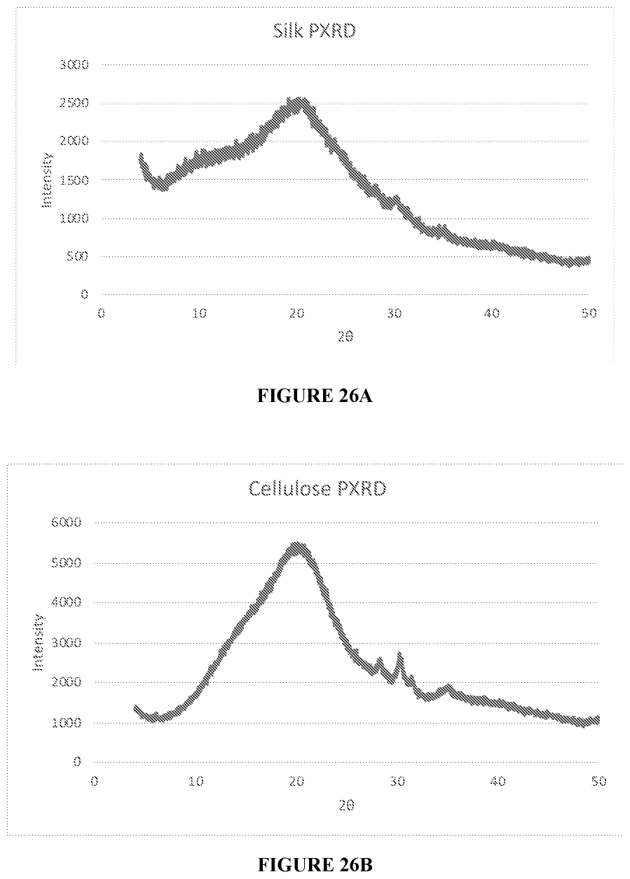

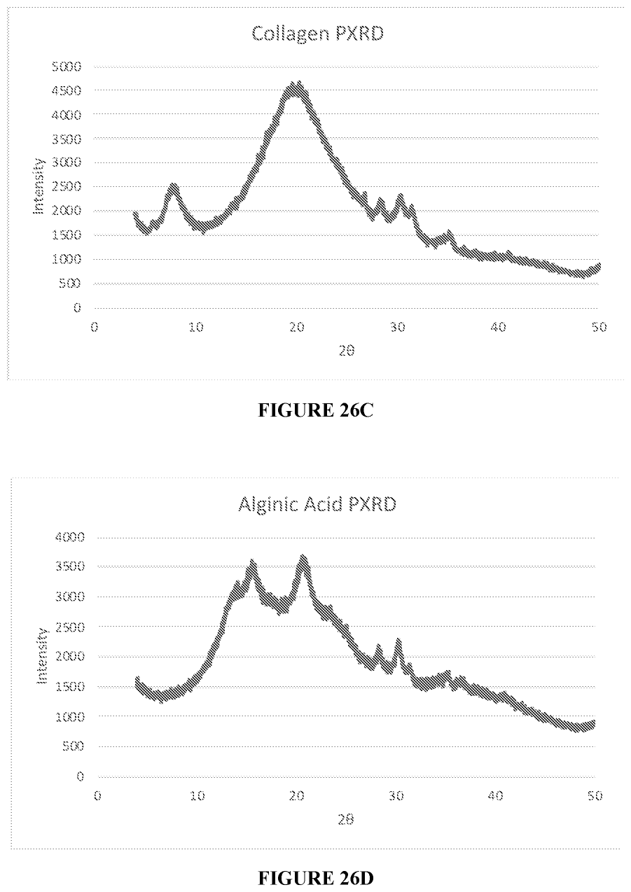

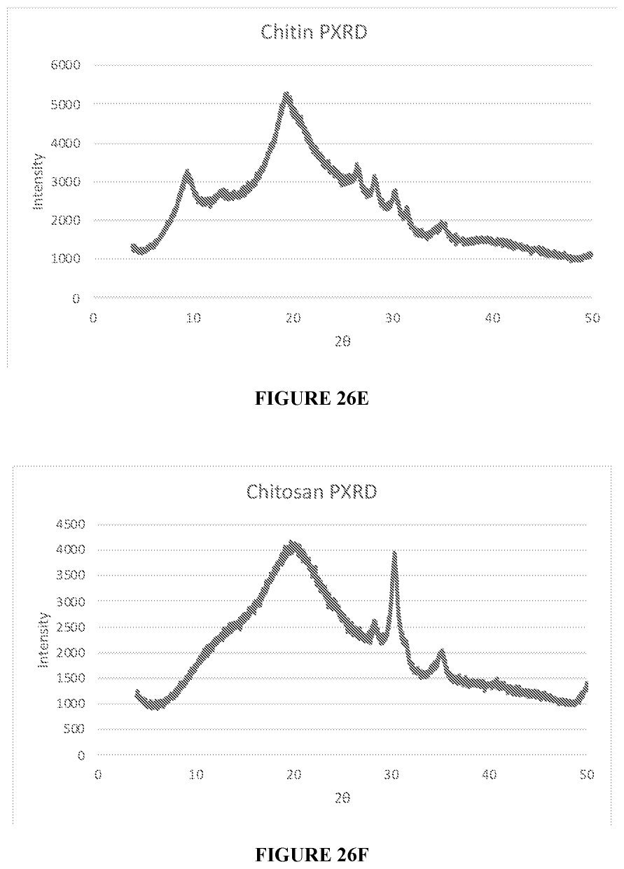

In embodiments, the biopolymer suspension or composition is characterized by Power X-Ray Diffraction (PXRD) pattern(s) as depicted in any one of FIGS. 26A to 26F (e.g., Example 8).

In embodiments, the biopolymer suspension or composition is characterized by Dynamic Light Scattering (DLS) measurements like those reported in Table 3 (e.g., Example 9).

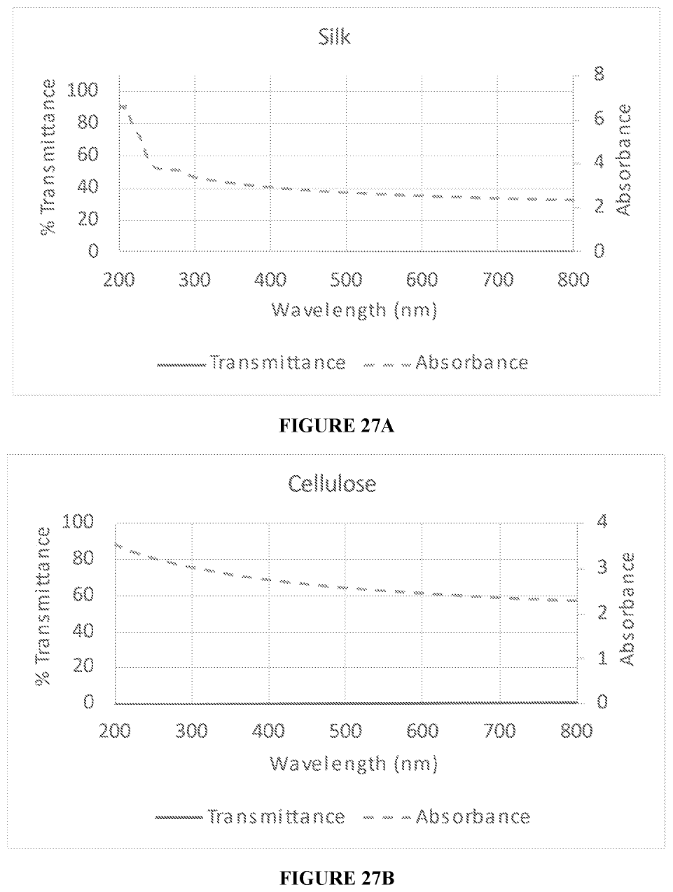

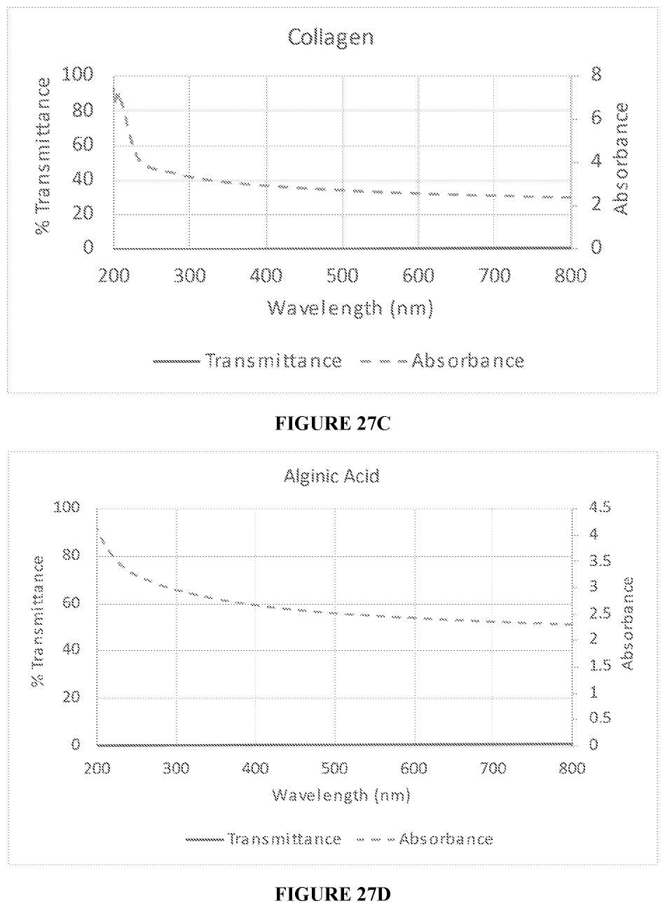

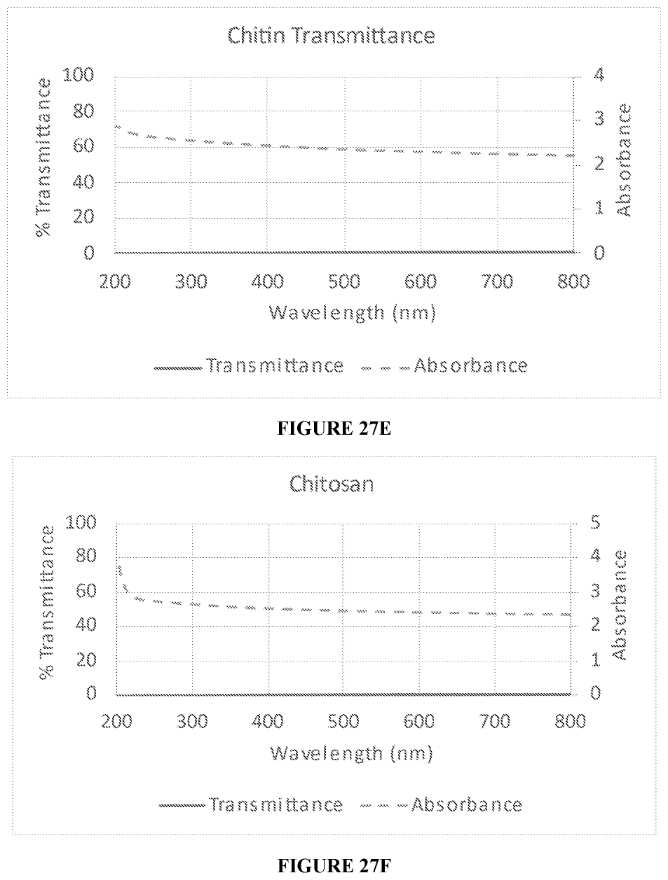

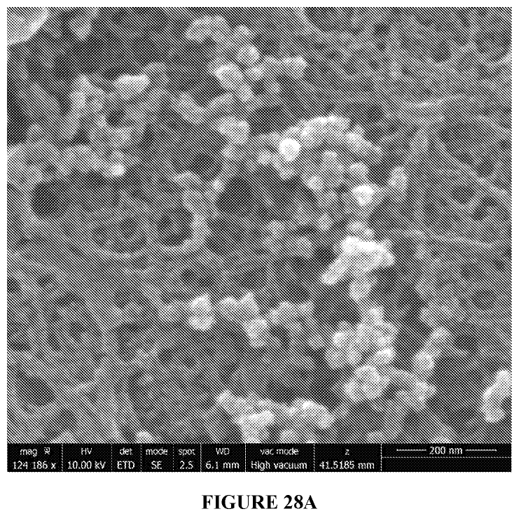

In embodiments, the biopolymer suspension or composition is characterized by a transmittance spectrum as shown in any one of FIGS. 27A to 27F (e.g., Example 10).

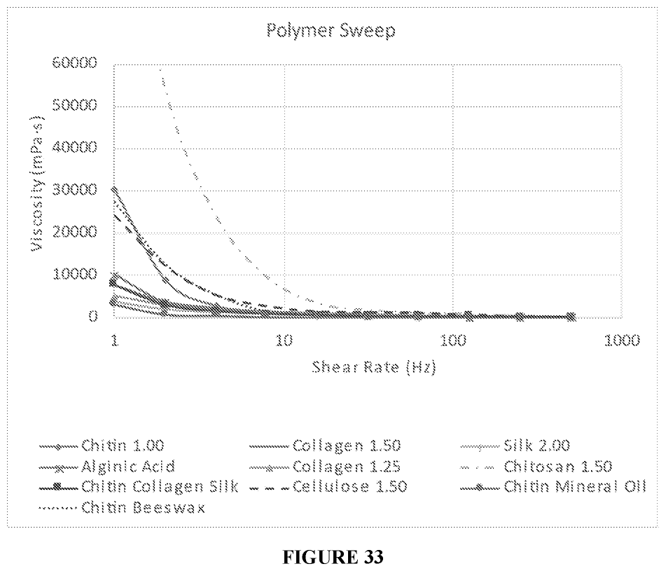

In embodiments, the biopolymer suspension or composition is characterized by a sweep suspension test as reported in Table 5 (e.g., Example 12) or as depicted in FIG. 33 (e.g., Example 13)

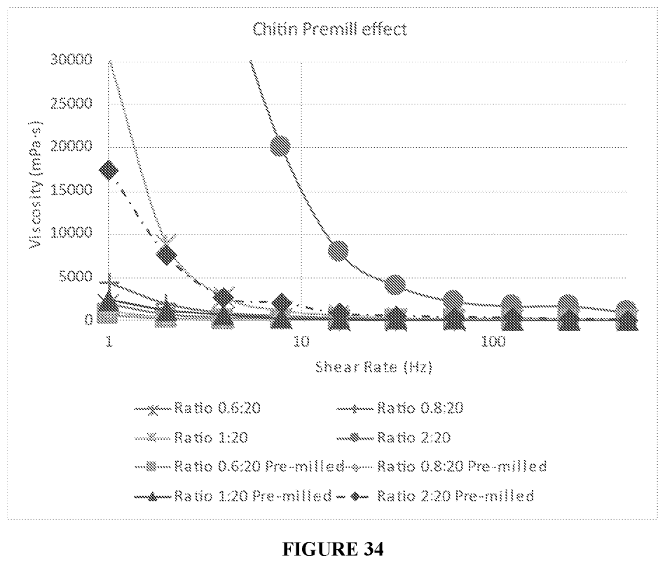

In embodiments, the biopolymer suspension or composition is characterized by a rheological behaviour as depicted in FIG. 34 (e.g., Example 14).

Advantageously, the stable homogeneous suspension of the invention is very stable, i.e., the biopolymer (e.g., fibers, spherical bodies) does not settle at the bottom. In embodiments the insoluble and/or semi-soluble biopolymer(s) remains in suspension for at least 1 week, or at least 1 month, or at least 6 months, or at least 12 months, or at least 18 months, or at least two years, or at least three years or more.

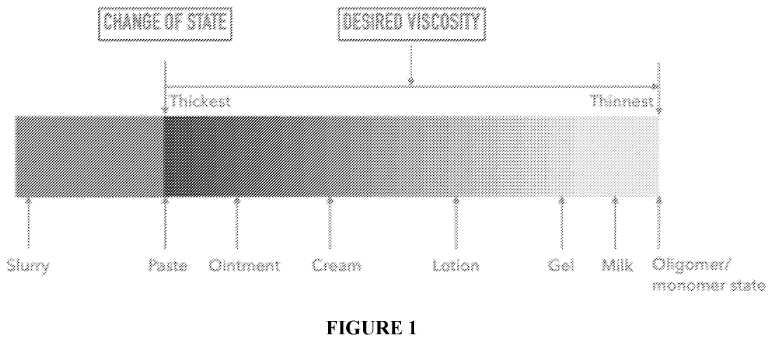

As illustrated in FIG. 1 and explained herein after, it is possible to vary the viscosity of the compositions and suspensions according to the present invention. Indeed, the viscosity can be varied such that biopolymer composition has the viscosity of what is generally referred to as a paste, an ointment, a cream, a lotion, a gel or a milk. In embodiments the stable homogeneous suspension comprises a viscosity of about 25 mPa to about 85 000 mPa.

In embodiments the biopolymer composition or suspension is substantially pure and it consists essentially of the biopolymer(s) and polar solvent(s) (e.g., water). Therefore, such composition or suspension is advantageously substantially free from any chemical residues and other chemicals that may be required in the prior art to produce suspensions comprising biopolymers. As used herein, “substantially free from chemical residues” means that chemical compounds, such as acids, bases, reactive chemicals, organic salts and/or inorganic salts, surfactants, dispersing agents (e.g., Twin 80™), a silanizing reagent, acrylamide, etc. are totally absent or merely present in undetectable or trace amounts in the final composition or final suspension. In embodiments, the biopolymer(s) will constitute at least 98%, or at least 99% or at least 99.9% or at least 99.99% by weight of the organic compounds in the biopolymer composition or suspension, i.e., the biopolymer composition or suspension will contain less than 2% or less than 1%, less than 0.1%, or less than 0.01%, or less than 0.001% by weight of organic components other than the biopolymer(s) or degradation product(s).

The biopolymer composition or suspension may also comprise one or more additives. A not limitative list of additives includes, but is not limited to, preservatives, stabilizers and emulsifiers (e.g., Cetyl alcohol, Glyceryl stearate, Soy butter, PC90, Tara Gum, PSC3, PEG, Guar, Xantham gum, Agarose, Sodium Hyaluronate, Tween 80™, Glycerol (humectant)), thickeners, dyes, powders (e.g., mica, pigment, chalk), inks, colorants, fragrances, essential oils, extracts (e.g., plant extract(s) such as aloe vera), vitamins (e.g., ascorbic acid), acids (e.g., acetic acid, citric acid, stearic acid), oils (cocoa butter, emu oil, olive oil, shea butter, silicone oil, mineral oil), metal oxides (e.g., zinc oxides), salts (e.g., sea salts, sodium lactate), honey, clay, propenyl glycol, polyethylene glycol, dry ingredient (e.g., rose petal powder, orange peel powder, chamomile flowers, calendula petals, etc.), allantoin, acetylglucosamine (GlcNAc), waxes (e.g., beeswax), peptides and proteins, pharmaceutical compounds (e.g., N-Acetyl Glucosamine, lidocaine, capsaicin, baclofen, ketamine, methylsulfonylmethane, orphenadrine, tetracaine, amitriptyline, bupivacaine, cyclobenzaprine, doxepin, gabapentin, guaifenesin, acetaminophen, ibuprofen, naproxen, diclofenac, meloxicam, piroxicam, ketoprofen, any NSAIDs), sugars (e.g. glucose, fructose, galactose, etc.), monomers of any of cellulose, starch, chitin, chitosan, alginic acid, collagen, silk, etc. The additive(s) may be added prior, during and/or after the step of high-shearing conditions and/or high mechanical energy.

In embodiments, the additive or stabilizer is selected from the following stabilizers: Agar, sodium alginate, carrageenans, guar, konjac, tragacanth, locust bean gum, psyllium, tara gum, fenugreek gum, xanthan gum, abietic acid, acetyl mannosylerythritol lipid, acrylamide/sodium acryloyldimethyltaurate copolymer, acrylates/aminoacrylates/C10-30 alkyl peg-20 itaconate copolymer, acrylates/C10-30 alkyl acrylate crosspolymer, acrylates/C5-8 alkyl acrylate copolymer, acrylates/stearyl methacrylate copolymer, acrylates/vinyl isodecanoate crosspolymer, acrylates/vinyl neodecanoate crosspolymer, acrylic acid/stearyl acrylate copolymer, acrylic acid/stearyl methacrylate/dimethicone methacrylate copolymer, bis-acryloyl poloxamer, alcaligenes polysaccharides, alcohols C9-11, allyl methacrylates crosspolymer, sweet almond oil polyglyceryl-4 esters, aluminum behenate, aluminum caprylate, aluminum dicetyl phosphate, aluminum dilinoleate, aluminum dimyristate, aluminum distearate, aluminum isostearate, aluminum isostearates/laurates/palmitates, aluminum isostearates/laurates/stearates, aluminum isostearates/myristates, aluminum isostearates/palmitates, aluminum isostearates/stearates, aluminum lanolate, aluminum monostearate, aluminum myristate, aluminum myristates/palmitates, aluminum/magnesium hydroxide stearate, ammonium acryloyldimethyltaurate/steareth-25 methacrylate crosspolymer, ammonium acryloyldimethyltaurate/steareth-8 methacrylate copolymer, ammonium acryloyldimethyltaurate/vinyl formamide copolymer, ammonium alginate, ammonium phosphatidyl rapeseedate, ammonium polyacryloyldimethyl taurate, ammonium shellacate, amodimethicone glycerocarbamate, AMP-C8-18 perfluoroalkylethyl phosphate, aphanothece sacrum polysaccharide, arachidyl alcohol, astragalus gummifer gum, astragalus gummifer root extract, avocadamide DEA, babassu acid, crosslinked bacillus/glucose/sodium glutamate ferment, dextro,laevo-batyl alcohol, hydrolyzed beeswax, synthetic beeswax, behenyl alcohol, bentonite, benzalkonium montmorillonite, benzalkonium sepiolite, bittern emulsion stabilising, brassica alcohol emollients, brassicyl isoleucinate esylate, butendiol/vinyl alcohol copolymer, butoxyhydroxypropyl cetyl hydroxyethylcellulose, butter decyl esters, butyl acrylate/isopropylacrylamide/peg-18 dimethacrylate crosspolymer, butyl babassuate, butylene glycol cocoate, butylene glycol isostearates, C1-5 alkyl galactomannan, C12-13 alcohols, C12-14 sec-pareth-3, C12-14 sec-pareth-5, C12-14 sec-pareth-7, C12-14 sec-pareth-8, C12-14 sec-pareth-9, C12-14 sec-pareth-12, C12-14 sec-pareth-15, C12-14 sec-pareth-20, C12-14 sec-pareth-30, C12-14 sec-pareth-40, C12-14 sec-pareth-50, C12-15 alcohols, C12-16 alkyl peg-2 hydroxypropyl hydroxyethyl ethylcellulose, C12-18 alkyl glucoside, Hydrogenated C12-18 triglycerides, C14-15 alcohols, C14-18 glycol, C14-22 alcohols, C15-18 glycol, bis-C16-18 alkyl glyceryl undecyl dimethicone, C18-22 alkyl peg-25 methacrylate/diethylaminoethyl methacrylate copolymer, C18-30 glycol, C18-38 alkyl hydroxystearoyl stearate, C20-22 alcohols, C20-30 glycol, C20-40 alcohols, C20-40 alkyl crylene, C22-24 pareth-33, bis-C24-28 hydroxyalkyl olivoyl glutamate, C28-52 olefin/undecylenic acid copolymer, C30-50 alcohols, calcium carboxymethyl cellulose, calcium carrageenan, calcium laurate, calcium myristate, calcium polyglutamate crosspolymer, calcium potassium carbomer, calcium saccharate, calcium starch octenylsuccinate, calcium stearate, callitris quadrivalvis resin, candelilla cera, candelilla wax, candelilla/jojoba/rice bran polyglyceryl-3 esters, Cannabis sativa seed oil glycereth-8 esters, caprylyl dimethicone ethoxy glucoside, caprylyl/capryl wheat bran/straw glycosides, carbomer 934, carboxymethyl cellulose acetate butyrate, carboxymethyl hydroxyethyl cellulose, carboxymethyl hydroxypropyl guar, carnauba wax, carthamus tinctorius oleosomes, hydrogenated castor oil behenyl esters, castor oil phosphate, hydrogenated castor oil stearyl esters, hydrogenated castor oil/sebacic acid copolymer caprate/caprylate, cellulose acetate propionate carboxylate, hydrolyzed cellulose gum, cellulose microcrystalline, ceramide NS/peg-8/succinic acid copolymer, ceratonia siliqua gum, ceresin, ceteareth-6 olivate, cetostearyl alcohol, cetyl alcohol, cetyl dimethicone peg-7 acetate, cetyl dodecenylsuccinate, cetyl hydroxyethyl cellulose, cetyl peg/ppg-7/3 dimethicone, bis-cetyl/peg-8 cetyl peg-8 dimethicone, chitosan lauramide succinimide, chitosan lauroyl glycinate, cholesterol/hdi/pullulan copolymer, citrus aurantium dulcis peel extract, citrus aurantium sinensis fiber, cocamide, cocamide DEA, cocamide MEA, cocamide MIPA, cocamidopropyl lauryl ether, cocoa butter glyceryl esters, coconut alcohol, coconut oil methylpropanediol esters, hydrolyzed corn starch hydroxyethyl ether, cyamopsis tetragonoloba gum, decyl castorate, decyl glucoside, decyl hempseedate, 7-dehydrocholesterol, dehydroxanthan gum, dicapryl sodium sulfosuccinate, diethylene glycol/hydrogenated dimer dilinoleic acid copolymer, digalactosyl glyceryl linoleate/palmitate/oleate, diglycerin/dilinoleic acid/hydroxystearic acid copolymer, dihydrolanosterol, dihydroxyethyl cocamine oxide, dihydroxyethyl lauramine oxide, diisotridecyl lauroyl glutamate, dilauryl maleate/C20 olefin copolymer, dimaltosyl cyclodextrin, hydrogenated dimer dilinoleyl/dimethylcarbonate copolymer, dimethicone crosspolymer, dimethicone ethoxy glucoside, dimethicone/lauryl dimethicone/bis-vinyldimethicone crosspolymer, dimethicone/peg-15 crosspolymer, dimethyl capramide, dimethyl cocamine, dimethyl lauramine isostearate, dioleyl phosphate, dipropylene glycol isobornyl ether, dodecylhexadecanol, bis-ethoxydiglycol cyclohexane 1,4-dicarboxylate, ethyl hydroxyethyl cellulose, bis-ethyl ppg-behenate dimonium methosulfate, bis-(ethyl ppg-3 behenate) dimonium methosulfate, ethylene vinyl acetate copolymer, ethylene/acrylic acid copolymer, ethylene/sodium acrylate copolymer, feruloyl soy glycerides, perfluorocyclohexylmethanol, perfluoroheptane, perfluoromethylcyclohexane, perfluoromethyldecalin, ghatti gum, glucose pentaacetate, alpha-dextro-glucose pentaacetate, glycereth-7 malate, glycereth-8 hydroxystearate, glycereth-7 benzoate, hydrogenated glyceryl abietate, glycol cetearate, acetylated glycol stearate, glycosyl trehalose, grape seed oil glycereth-8 esters, grape seed oil polyglycerin-6 esters, maleated hexene/propylene copolymer, hydroxquinoline sulfate, hydroxyapatite, hydroxybutyl methyl cellulose, bis-hydroxyethoxypropyl dimethicone beeswax esters, bis-hydroxyethoxypropyl dimethicone isostearate, hydroxyethyl acrylate/sodium acryloyldimethyl taurate copolymer, hydroxyethyl cellulose, hydroxyethyl isostearyloxy isopropanolamine, hydroxypropyl cellulose, hydroxypropyl guar gum, hydroxypropyl methyl cellulose, hydroxypropyl xanthan gum, hydroxypropyltrimonium inulin, hydroxypropyltrimonium xanthan gum, hydroxystearic/linolenic/linoleic polyglycerides, hydroxystearic/linolenic/oleic polyglycerides, inulin lauryl carbamate, synthetic japan wax, jojoba oil glycereth-8 esters, hydrogenated lanolin alcohol, lanolinamide DEA, lauryl alcohol, lauryl alcohol diphosphonic acid, lauryl dodecenylsuccinate, lauryl/myristyl wheat bran/straw glycosides, hydrogenated lime seed oil, magnesium alginate, maltitol laurate, maltodextrin, methoxy peg-22/dodecyl glycol copolymer, methoxy peg/ppg-25/4 dimethicone, methyl cellulose, methyl vinyl ether-maleic anhydride copolymer, montmorillonite, myrist/palmitamidobutyl guanidine acetate, myristyl alaninate, myristyl alcohol, oleic/linoleic/linolenic polyglycerides, olive alcohol, hydrogenated olive oil caprylyl esters, hydrogenated olive oil cetyl esters, hydrogenated olive oil decyl esters, hydrogenated olive oil hexyl esters, hydrogenated olive oil lauryl esters, hydrogenated olive oil myristyl esters, hydrogenated olive oil stearyl esters, hydrogenated orange seed oil, ozokerite, palm kernel amide DEA, palm kernel amide MEA, palm kernelamide MIPA, palmamide DEA, palmamide MEA, palmamide MIPA, peanutamide MEA, peanutamide MIPA, pectin, tris(peg-2 phenylalanylcarboxamido) cyclohexane, peg-2 tallowamide DEA, peg-4 peg-12 dimethicone, peg-5 pentaerythrityl dimethylol propionate-2 dendrimer, peg-5 pentaerythrityl dimethylol propionate-3 dendrimer, peg-5 pentaerythrityl dimethylol propionate-4 dendrimer, peg-7 propylheptyl ether, peg-7m, peg-8 dimethicone/polysorbate 20 crosspolymer, peg-8 propylheptyl ether, peg-9m, peg-12 carnauba, peg-12 glyceryl linoleate, peg-14m, peg-20m, peg-23m, peg-65m, peg-90m, peg-100/IPDI copolymer, peg-114 polylactic acid, peg-115m, peg-160m, peg-180m, peg-400, peg-45/dodecyl glycol copolymer, peg-450, peg-500, peg/ppg-10/3 oleyl ether dimethicone, peg/ppg-100/70 tocopheryl ether, bis-peg/ppg-15/5 dimethicone, peg/ppg-18/18 isostearate, peg/ppg-18/18 laurate, peg/ppg-2/5 tocopheryl ether, peg/ppg-20/23 dimethicone, bis-peg/ppg-20/5 peg/ppg-20/5 dimethicone, peg/ppg-2000/200 copolymer, peg/ppg-23/6 dimethicone, peg/ppg-30/10 tocopheryl ether, peg/ppg-5/10 tocopheryl ether, peg/ppg-5/20 tocopheryl ether, peg/ppg-5/30 tocopheryl ether, peg/ppg-50/20 tocopheryl ether, peg/ppg-6/4 dimethicone, peg/ppg-70/30 tocopheryl ether, peg/ppg-8/3 laurate, pentadecyl alcohol, petrolatum wax microcrystalline, phosphatidic acid, phosphatidyl serine, phosphatidylglycerol, pineamidopropyl betaine, poly C10-30 alkyl acrylate, polyacrylate crosspolymer-4, polyacrylate crosspolymer-6, polyacrylate crosspolymer-11, polyacrylate crosspolymer-14, polyacrylate-10, polyacrylate-11, polyacrylate-27, polyacrylate-28, polyacrylic acid, polyester-14, polyester-15, polyethylene/isopropyl maleate/MA copolyol, polyglyceryl-2 diisostearate/ipdi copolymer, polyglyceryl-3 sunflowerseedate/citrate crosspolymer, polyglyceryl-4 diisostearate/polyhydroxystearate/sebacate, polyglyceryl-6 behenate, polypropanediol, polypropylene terephthalate, polyquaternium crosspolymer-2, polyquaternium-65, polyquaternium-83, polyquaternium-102, polyquaternium-103, polysilicone-25, polyurethane-29, polyvinyl acetate, polyvinyl pyrrolidone, potassium alginate, potassium behenoyl hydrolyzed rice protein, potassium behenoyl hydroxyproline, potassium carbomer, potassium carrageenan, potassium stearoyl hydrolyzed rice protein, potassium undecylenoyl alginate, potassium undecylenoyl carrageenan, potassium undecylenoyl hydrolyzed corn protein, potassium undecylenoyl hydrolyzed soy protein, potassium undecylenoyl hydrolyzed wheat protein, hydrolyzed potato tuber extract, ppg-4 jojoba alcohol, ppg-4 laureth-2, ppg-4 laureth-5, ppg-6-laureth-3, ppg-20 tocophereth-5, ppg-10 jojoba acid, ppg-2-buteth-2, propyl ester of PVM/MA copolymer, prunus amygdalus dulcis oil unsaponifiables, Pseudozyma epicola/Camellia sinensis seed oil/glucose/glycine soja meal/malt extract/yeast extract ferment filtrate, PVP montmorillonite, PVP/decene copolymer, pyrus malus fiber, quaternium-90 sepiolite, rhamnolipids, sclerotium gum, hydrogenated sesame seed oil, sesquiethoxytriethanolamine, sesquioctyldodecyl lauroyl glutamate, shea butter glycerides, silica dimethyl silylate, silica silylate, beta-sitosterol, sodium acrylate/acryloyldimethyltaurate/dimethylacrylamide crosspolymer, sodium acrylate/sodium acryloyldimethyl taurate copolymer, sodium acrylate/sodium acryloyldimethyl taurate/acrylamide copolymer, sodium acrylate/vinyl alcohol copolymer, sodium acrylates/vinyl isodecanoate crosspolymer, sodium acryloyldimethyl taurate/acrylamide/VP copolymer, sodium acryloyldimethyltaurate/VP crosspolymer, sodium arachidate, sodium C4-12 olefin/maleic acid copolymer, sodium carbomer, sodium carboxymethyl cellulose, sodium carboxymethyl dextran, sodium carboxymethyl starch, sodium carrageenan, sodium cellulose sulfate binding, sodium cocoyl barley amino acids, sodium cocoyl/stearoyl (alanine/arginine/asparagine/aspartic acid/glutamic acid/glutamine/glycine/histidine, sodium cyclodextrin sulfate, sodium dextrin octenylsuccinate, sodium laneth sulfate, sodium polyacrylate, sodium polyacrylate starch, sodium polyacryloyldimethyl taurate, sodium polygamma-glutamate, sodium polygamma-glutamate crosspolymer, sodium polyglutamate crosspolymer, sodium polymethacrylate, sodium polynaphthalenesulfonate, sodium polystyrene sulfonate, sodium starch octenyl succinate, sodium styrene/MA copolymer, sodium tocopheryl phosphate antioxidants, sodium trehalose octenylsuccinate, sodium/TEA-undecylenoyl alginate, sodium/TEA-undecylenoyl carrageenan, sorbitan palmate, soy protein phthalate, soyamide DEA, sparassis crispa extract, starch hydroxypropyltrimonium chloride, iso-steareth-200 palmitate, stearic acid, stearyl alcohol, stearyl glycol, stearyl vinyl ether/MA copolymer, sterculia urens gum, stigmasteryl chloride, stigmasteryl nonanoate, stigmasteryl succinate, styrene/ma copolymer, sucrose polypalmate, sunflower seed oil ethyl ferulate esters, sunflower seed oil polyglyceryl-10 esters, sunflower seed oil polyglyceryl-6 esters, tallow alcohol, tallow amide cosmetic agents, tamarindus indica seed gum, TEA-alginate, TEA-dextrin octenylsuccinate, tetradecyleicosanoic acid, tetradecyloctadecanoic acid, tetradecyloctadecyl behenate, tetradecyloctadecyl myristate, tetradecyloctadecyl stearate, tetrasodium etidronate, theobroma grandiflorum seed butter glyceryl esters, tocopheryl succinate methylglucamide, tremella fuciformis polysaccharide, triacontene/VP copolymer, 1-Tridecanol, tripropylene glycol, undeceth-40, undecylenoyl inulin, undecylenoyl xanthan gum, vinyl alcohol/vinylformamide copolymer, bis-vinyl dimethicone/dimethicone copolymer, bis-vinyldimethicone/peg-10 dimethicone crosspolymer, hydrogenated microcrystalline wax hydrotreated, welan gum, xanthan gum, zinc undecylenoyl hydrolyzed wheat protein.

In embodiments the biopolymer composition or suspension according to the invention satisfies ISO 11930 preservative effectiveness test that is a procedure for evaluating the antimicrobial protection of a product. This test has been written specifically for cosmetic products and it is quickly becoming the “go to” test method for evaluating the preservative effectiveness of cosmetics and personal care products. In embodiments the biopolymer composition or suspension according to the invention provides cosmetically useful antimicrobial protection against one or more strains of microorganisms including, but not limited to S. aureus, E. coli, P. aeruginosa, C. albicans, and A. brasiliensis.

As demonstrated in Example 6, insoluble and/or semi-soluble biopolymer may act as an emulsifier may advantageously serve as an emulsifier to a stable emulsion.

In embodiments the biopolymer composition or suspension is obtained by a process other than chemical processing. In embodiments the biopolymer compositions or suspensions according to the invention are obtained by submitting the biopolymer(s) and polar solvent(s) to high-shearing conditions, for instance high mechanical energy. In embodiments the high-shearing conditions and/or high mechanical energy is obtained by a process including, but not limited to mechanical shearing, sheer thinning, planetary ball milling, rolling mill, vibrating ball mill, tumbling stirred ball mill, horizontal media mill, colloid milling. As indicated hereinafter, the high-shearing conditions and/or high mechanical energy can be carried out for a duration, under parameters, under suitable conditions, etc. until a desirable change of state is obtained, e.g., change of color, a change in viscosity, a change from a slurry to a paste, ointment, cream, lotion, gel or milk, etc.

In embodiments the high-shearing conditions and/or high mechanical energy requires using a suitable device or apparatus including, but not limited to, ball miller (e.g., planetary ball miller, rolling miller, vibrating ball miller, tumbling stirred ball miller, horizontal media mill, colloid miller, a magnetic miller), a twin-screw extruder, a high-pressure homogenizer, a blade homogenizer, a stirring homogenizer, a disperser, a rotor-stator homogenizer, a high-shear mixer, a plowshare mixer, a dynamic mixer, a plough mixer, a turbine mixer, a speed mixer, an attrition miller, a sonicator, a tissue tearor, a cell lysor, a polytron, a ribbon agitator, a microfluidizer, and combinations thereof. In preferred embodiments, the present invention utilizes ball milling under wet conditions.

The desired properties of the compositions or suspensions according to the invention (e.g. physical and chemical properties, purity, presence or absence of added chemicals, etc.) may be characterized using any suitable methods or technique known in the art. Examples include, but are not limited to scanning electron microscopy (SEM) which characterizes particle size, rheology which characterizes thixotropy and sheer-thinning behaviour, X-ray diffraction (XRD) which characterizes crystallinity, Dynamic light scattering (DLS) which characterizes particle size distribution, Fourier transform infrared spectroscopy (FTIR) spectroscopy which can be used to obtain the infrared spectrum of absorption, emission, and photoconductivity of solid, liquid, and gas, solid-state nuclear magnetic resonance characterization (SSNMR) which can be used for study of amorphous materials, as well to detect different constituents present in the composition, atomic force microscopy (AFM), mass spectrometry which characterizes wet particle size, cryo-scanning electron microscopy (cryo-SEM) which characterizes wet/frozen particle size, liquid color analysis which characterizes color of the sample, etc.

### Processes for Obtaining Biopolymer Compositions and Suspensions

Additional aspects of the invention concern processes and methods for obtaining biopolymer compositions and suspensions as defined herein.

According to one particular aspect, the invention relates to a mechanical process for obtaining a biopolymer composition, the process comprising subjecting an insoluble and/or semi-soluble biopolymer to mechanical energy in presence of a polar solvent to obtain a stable homogeneous suspension of the insoluble and/or semi-soluble biopolymer(s).

Without being bound by any theory, as indicated hereinbefore, it is proposed that the mechanical energy results in a shearing and/or sheer thinning of the biopolymer. The mechanical energy may also lead to a certain “degradation” or “transformation” of the multimeric biopolymer into smaller monomeric units.

Accordingly, another particular aspect of the invention relates to a process for obtaining a biopolymer composition, the process comprises subjecting an insoluble and/or semi-soluble biopolymer to high-shearing conditions in presence of a polar solvent until a change of state is observed and a stable homogeneous suspension of the insoluble and/or semi-soluble biopolymer is obtained.

In embodiments the insoluble biopolymer is selected from chitin, chitosan, cellulose, hemicellulose, lignin, amylose, actin, fibrin, collagen, silk, fibroin, keratin, wool, and mixtures thereof. In embodiments the semi-soluble biopolymer is selected from gelatin, pectin, starch, amylopectin, agarose, alginic acid, alginate, hyaluronic acid, RNA, DNA, xanthan gum, guar gum, carageenan, latex, polymannans, suberin, cutin, cutan, and mixtures thereof.

In embodiments the insoluble or semi-soluble biopolymer is obtained from fungi and mushrooms. In embodiments the insoluble or semi-soluble biopolymer is obtained from plant materials including, but not limited to, roots, tubers, leaves, petals, seeds, fruits, etc. In particular embodiments, the biopolymer suspension or biopolymer composition according to the present invention is obtained by subjecting to high-shearing conditions and/or high mechanical energy plant materials from one or more of the following: abscess root, agai, alder buckthorn, alfalfa, aloe vera, amargo, arnica, asafoetida, ashoka tree, ashwagandha, asthma-plant, astragalus, avaram senna, balloon flower, barberry, basil, bay laurel, bay leaf, belladonna, Benjamin, bhringraj, bilberry, bitter leaf, bitter-wood, black cohosh, blessed thistle, blue snakeweed, blueberries, borage, burdock, calendula, camelina, cannabis, caraway, carrot, cat's claw, cayenne, celery, centella, chamomile, chaparral, charcoal-tree, chasteberry, chickweed, chicory, chili, cinchona, cinnamon, clove, clover, cocoa, coffee senna, comfrey, coriander, cornflower, cranberry, cucumber, cumin, daisy, dandelion, deodar, digitalis, dock, dogwood, dong quai, drumstick tree, echinacea, elderberry, elderflower, elecampane, ephedra, eucalyptus, eyebright, false sowthistle, fenugreek, fever root, feverfew, field scabious, flaxseed, foxglove, fumitory, galanga, ganja, garden angelica, garlic, geranium, ginger, ginkgo, ginseng, goldenseal, gotu kola, grape, ground-ivy, guava, gum Arabic, hawkweed, hawthorn, heena, helichrysum, hemp, henna, hepatica, hibiscus, hollyhock, hoodia, horse chestnut, horsetail, Humulus lupulus, hyssop, inchplant, jasmine, kalonji, kanna, kapurkachir, karvy, kava, khat, konjac, kratom, lady's mantle, laurustinus, lavender, lemon, lemon balm, lemon citrus, lichen, licorice root, lilly, liquorice, lotus, lungwort, madreselva, magnolia-bark, mallow, manjistha, marigold, marijuana, marsh-mallow, melon, milk thistle, minnieroot, mint, mistletoe, moringa, mullein, myrrh, neem, nettle, nigella, noni, oat, opium poppy, orange, oregano, orris, pansy, papaya, passion flower, peppermint, plantai, plantain, platycodon, poppy, primrose, purple coneflower, robert geranium, rose, rosemary, saffron, sage, salae, sandalwood, saponaria, savory, sea buckthorn, shikakai, shoreline purslane, small-leaved linden, snapdragon root, snowdrop, soap wort, speedwell, St. john's wort, star anise, summer snowflake, sunflower, sweet flag, syrian rue, tea, tea tree oil, thyme, tomato, tulsi, turmeric, umckaloabo, valerian, velvetleaf, verbena, veronica, vetiver, violet, wafer ash, wahoo, water germander, water-plantain, watercress, wheat germ, wheatgrass, white buttercup, white snakeroot, white willow, wild cherry bark, witch-hazel, yarrow, yellow lady's slipper, yerba mate, yerba santa, and zedoary.

In embodiments the polar solvent is selected from polar protic solvents, polar aprotic solvents and mixture thereof. The polar solvent may be an aqueous solvent. The present invention encompasses the use of more than one solvent in the same or in different categories. Envisioned examples of polar protic solvents, polar aprotic solvents and aqueous solvent are as defined hereinbefore.

Various sources of biopolymer may be used and the present invention is not limited to particular sources of materials. For instance, suitable sources of chitin may include, but are not limited to, green plants, algae, and fungi. Suitable sources of chitin and chitosan may include, but are limited to, fungi, crustaceans (e.g. crabs and shrimps) and insects.

In embodiments the biopolymer(s) which is subjected to the mechanical energy or to high-shearing conditions is a powder of pure biopolymer materials (e.g., Sigma). In embodiments, the biopolymer(s) is a dry biopolymer (e.g., not wet and/or not swollen). In embodiments, the biopolymer(s) is a dry biopolymer that is not a wet biopolymer that has been left to dry (such wet then dried biopolymer typically looks porous in SEM).

In embodiments, the biopolymer is a biopolymer other than wet chitin, pre-wet chitin and/or swollen chitin, like chitin extracted from shells and exposed to acid for demineralization and to a base for deproteinization. In embodiments, the biopolymer is a biopolymer that was originally in a dry form and thereafter rendered wet, pre-wet and/or swollen prior to being submitted to mechanical energy/high-shearing conditions.

It may also be envisioned according to the present invention, to use “less pure” extracts of biopolymers, such as extracts obtained from prawn shells, crab shells, shrimp shells, lobster shells, insects, fungus, woods, plant cellulose, etc.

In embodiments, the biopolymer composition or suspension is achieved without the use of catalysts or other chemical additives. In embodiments, the processes of the invention do not require chemical processing, which is different from existing methods which typically require chemicals residues such as acids, bases, reactive chemicals, and/or organic salts and/or inorganic salts to produce biopolymers suspensions. Therefore, the processes of the invention may provide biopolymer compositions and suspension which are substantially free from any chemicals, additives, etc. as defined hereinabove. Avoiding chemicals is advantageous to obtain biopolymer compositions and suspensions that are substantially pure, natural, biocompatible, biodegradable and/or free of toxic ingredients.

In embodiments the high-shearing conditions and/or high mechanical energy is obtained by a process including, but not limited to mechanical shearing, sheer thinning, planetary ball milling, rolling mill, vibrating ball mill, tumbling stirred ball mill, horizontal media mill, colloid milling.

Whenever necessary, or preferred, the biopolymer materials used in the suspension process may be altered prior to being subjected to the mechanical energy or to high-shearing conditions. Examples of possible alterations include, but are not limited to, cutting with scissors, grinding with a blade grinder, freeze-thawing, and/or dry ball milling, etc. to reduce particle size.

In embodiments the high-shearing conditions and/or high mechanical energy requires using a suitable device or apparatus including, but not limited to, ball miller (e.g., planetary ball miller, rolling miller, vibrating ball miller, tumbling stirred ball miller, horizontal media mill, colloid miller), a twin-screw extruder, a high-pressure homogenizer, a blade homogenizer, a stirring homogenizer, a disperser, a rotor-stator homogenizer, a high-shear mixer, a plowshare mixer, a dynamic mixer, a plough mixer, a turbine mixer, a sonicator, a tissue tearor, a cell lysor, a polytron, a ribbon agitator, a microfluidizer, and combinations thereof.

In one particular embodiment the process is carried out using a vertical planetary mill (e.g., Tencan XQM-2A™) with 100 mL capacity zirconia jars and 10 mm diameter zirconia balls. Other types of balls (e.g., 5 mm to 15 mm) and other jar sizes (i.e., 250 mL) may also be used. In one particular embodiment the process is carried out using a Flacktek™ speedmixer (DAC 330-11 SE) with 40 mL zirconia jar with 5 mm diameter zirconia balls or zirconia rings. In one particular embodiment the process is carried out using a 1.5 L Supermill Plus™ using 1.4-1.7 mm zirconia beads.

The present invention encompasses different ways to use ball millers including, but not limited to unidirectional milling continuous (no pausing), unidirectional milling with cyclical pauses (e.g., at either 10, 20, or 30 minutes), alternating milling direction with cyclical pauses (e.g., at either 10, 20, or 30 minutes), etc. In embodiments, the method comprises alternating milling wherein the biopolymer is milled for a certain period of time (e.g., 10 min, 15 min, or 20 min, or 30 min or more) followed by a short pause (e.g., 30 s, or 1 min, or 2 min, or 5 min, or 10 min, or 15 min or more) then milling in the opposite direction for a certain period of time (e.g., 10 min, 15 min, or 20 min, or 30 min, or more) for a total of 1 hour, or 2 hours, or 3 hours, or 5 hours, or 10 hours, or 12 hours, or 15 hours, or more.

In particular embodiments, biopolymer compositions and suspensions in accordance with the present invention are obtained using a particular protocol referred herein as the “10+1 Alt method”. This method comprises milling of the biopolymer for a certain period of time (e.g., 10 min) followed by a short pause (e.g., one min) then milling in the opposite direction for a certain period of time (e.g., 10 min) for a total of 1 hour, or 2 hours, or 3 hours, or 5 hours, 10 hours, or 12 hours. Uses of that method are described in Examples 8 to 27.

Advantageously, the viscosity of the compositions/suspensions can be altered by varying the high-shearing conditions and/or mechanical energy to which the biopolymer(s) are submitted. These conditions can be adjusted to obtain a stable homogeneous suspension (e.g., a stable colloidal homogeneous suspension) having a desired viscosity. For instance, as illustrated in FIG. 1, the viscosity can be varied such that biopolymer composition or suspension has the decreasing viscosity of a paste, an ointment, a cream, a lotion, a gel or a milk. Typically, providing more mechanical energy will increase the shearing and will reduce accordingly the viscosity of the end product.

Exemplary conditions or parameters that can be varied include, but are not limited to, speed (e.g., rotations per minute (RPM)), vessel size, ball quantity, ball size, vessel media, ball media, processing time, processing cycles, and batch size, ratio of ingredients (e.g., biopolymer:solvent weight ratio), etc.

In embodiments the biopolymer and aqueous solvent are in a biopolymer:solvent weight ratio of about 0.2:20 to about 10:20, or about 0.5:20 to about 3:20, or about 0.75:20, or about 1.0:20, 1.25:20. or about 1.5:20.

In embodiments the mechanical energy or high-shearing conditions are carried out until observation of a change of color. In embodiments such change of color comprises a change from a clear solution with a powder deposit to an opaque off-white homogeneous suspension having the viscosity of a thick paste (see FIG. 1 and Table 1). In one particular embodiment, the method comprises providing a specific mechanical energy of at least 0.4 to 500 W/kg for the total amount of material in the system (i.e., biopolymer(s)+solvent(s)+additive(s)).

In embodiments the mechanical energy or high-shearing conditions are carried out last for at least 15 min, or at least 30 min, or at least 45 min, or at least 60 min, or at least 90 min, or at least 2 hours, or at least 3 hours, or at least 5 hours. In embodiments the mechanical energy/high-shearing conditions is carried out for a period of time and for a duration leading to “degradation” of the multimeric biopolymer into smaller monomeric units. In embodiments the multimeric biopolymer is a polysaccharide and the monomeric unit is a monosaccharide. For instance, the multimeric biopolymer may be chitin and the monomeric unit N-Acetylglucosamine (GlcNAc).

Table 1 below provides non-limiting examples of desirable viscosity for the compositions/suspensions in accordance with the present invention.

TABLE 1
Examples of desired viscosities

| Desired Consistency | Exemplary viscosity (mPa · s) |
|---|---|
| paste | about 40 000 to about 100 000 |
| ointment | about 20 000 to about 50 000 |
| cream | about 1 500 to about 30 000 |
| lotion | about 800 to about 4 000 |
| gel | about 1 000 to about 40 000 |
| milk | about 20 to about 2000 |

As demonstrated in the examples, the processes of the invention may also be used to prepare stable emulsions comprising an oil and/or wax (Examples 6, 18 and 24), or comprising N-Acetyl Glucosamine (Example 16), or comprising additives such as the following additives were added to the cellulose suspension: Cetyl alcohol, Glyceryl stearate, Soy butter, PC90, Tara Gum, PSC3, PEG, Guar, Xantham gum, Agarose, Sodium Hyaluronate, Tween 80™ and Glycerol (Example 20), and with emulsifiers and preservatives (Example 27). Subjecting any of these compounds to mechanical energy or high-shearing conditions in presence of an insoluble and/or semi-soluble biopolymer as defined herein may result in a stable emulsion.

The processes of the invention may further comprise additional step(s), including one or more pre-treatment step(s) including, but not limited to, pre-milling, microwaving, freeze-thawing and steaming. In one particular embodiment the process comprises pre-milling the biopolymer in a dry environment to reduce the particular size and/or to obtain a fine powder (e.g., about less than 10 μm, or less than 5 μm, or less than 3 μm). In embodiments the pre-milling is carried out last for at least 15 min, or at least 30 min, or at least 45 min, or at least 60 min, or at least 90 min, or at least 2 hours, or at least 3 hours, or at least 5 hours, or at least 9 hours, or at least 12 hours. In another particular embodiment, the method comprises a pre-treatment step of freeze/thawing the biopolymer materials in water (e.g. one, two, three or more freeze-thaw cycle) prior to the high-shearing step such as milling. In another particular embodiment, the method comprises a pre-treatment step of microwaving and/or steaming the biopolymer materials prior to the high-shearing step such as milling. In another particular embodiment, the method comprises a pre-treatment step of pre-milling in propanol (e.g. isopropanol), the biopolymer materials prior to the high-shearing step such as milling.

In embodiments, the processes of the invention do not comprise and/or expressly exclude step(s) or technique(s) that may have been used in existing prior art methods to obtained biopolymer composition or suspensions, including, but not limited precipitation, centrifugation, filtration, sonication, homogenization (e.g., high-pressure homogenizer), lyophilisation, salinization, pulverization, stamping, swelling, mashing, cryogenic milling (e.g., liquid nitrogen in conjunction with a stirred ball mill), high shearing by stirring, mixing and/or with an impeller, microfluidization, embrittling, and attrition mill.

The processes of the invention may further comprise adding one or more additive(s) as defined herein prior, during and/or after the pre-treatment step, and/or prior, during and/or after the step of high-shearing and/or high mechanical energy.

It is within the knowledge of those skilled in the art to scale up production of the compositions and/or formulations of the invention in accordance with particular needs (e.g., to obtain at least 1.5 liter, or at least 15 liters, or at least 45 liters, or at least 75 liters, or at least 100 liters, or at least 150 liters or more). For instance, existing equipment for obtaining high-shearing conditions in larger volume include, but are not limited to, SuperMill Plus Media Mill™ 1.5 Liter, SuperMill Plus Media Mill™ 15 Liter, SuperMill Plus Media Mill™ 45 Liter, Batch Mill™ Model 100, Batch Mill™ Model 256, Double Planetary™ Mixer, Planetary PIus™ Mixer 7 Liter, Planetary PIus™ Mixer 150 Liter, Ram Press, Three Roll Mill, and SHRED/In-line Rotor Stator.

### Commercial Applications

The compositions and formulations of the invention may find numerous applications.

Another aspect of the invention relates to cosmetic compositions comprising the biopolymer compositions or suspensions as defined herein. In embodiments the cosmetic composition is formulated as a paste, an ointment, a cream, a lotion, a gel or a milk. In embodiments the cosmetic composition is formulated as a skin care composition, a hair care composition, a base composition, a vehicle composition, an anti-aging composition, a sunscreen blocking composition, a moisturizing composition, a makeup composition. Advantageously the cosmetic composition may comprise smaller monomeric units of a multimeric biopolymer, such as Acetylglucosamine (GlcNAc) and/or oligomers of NAGs and thus exhibits anti-aging and/or UV blocking properties.

The compositions and formulations of the invention is not limited to cosmetic applications as it may find numerous applications in various fields. For instance, it may be envisioned to use the compositions and formulations defined herein in seed coatings, surgical implant coatings, as food additives, paints, material additives, drug release platforms, etc.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following examples, which should not be construed as further or specifically limiting.

### EXAMPLES

This section provides non-limitative examples for obtaining stable homogeneous suspensions of biopolymers, in accordance with the present invention. Unless stated otherwise, the shearing processes were carried out using a vertical planetary mill (i.e., Tencan XQM-2A™) with 100 mL capacity zirconia jars and 10 mm diameter zirconia balls. Other types of balls (i.e., 5 mm to 15 mm) and other jar sizes (i.e., 250 mL) have also been used successfully.

### Example 1: Milling of Chitin with Water (Ratio 0.75:20)

Chitin was milled with water in a ratio of 0.75:20 w/w (chitin:water) for 3 hours at 670 RPM using 15 balls with diameter of 10 mm.

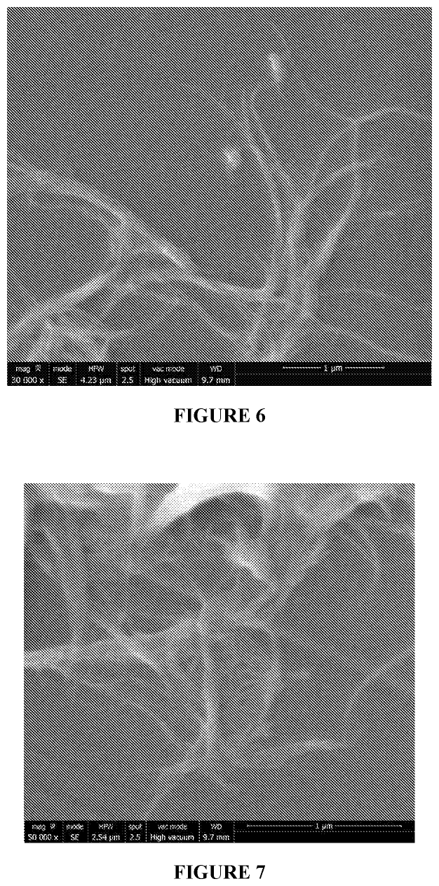

As illustrated in FIGS. 6, 7, 8 and 9, this produced separated fibers with widths on the nano-size with a low shear-rate viscosity of 11819 mPaS. The SEM images show partial fibrillation of the chitin fibers, where agglomeration exists and is represented by globular shapes as well as varied size fibers indicating amorphous regions persist as compared to more uniform, crystalline rods of nanochitin in acid processed chitin.

### Example 2: Milling of Chitin with Water (Ratio 1.5:20)

Chitin was milled with water in a ratio of 1.5:20 w/w (chitin:water) for 3 hours at 670 RPM using 30 balls with diameter of 10 mm, where the chitin was pre-milled for 3 hours at 670 RPM with 30 balls.

As illustrated in FIGS. 4, 5, 10 and 11, this produced separated fibers with widths on the nano-size with a low shear-rate viscosity of 6105 mPaS. This shows that pre-treatment can significantly decrease the viscosity of the suspension when compared to the dynamic modulus above. FIGS. 4 and 5 show the globular agglomeration of the chitin fibers upon drying for SEM preparation.

### Example 3: Samples A-F

Various types of samples were prepared to confirm robustness of the present invention under different conditions. Each sample was measured three times.

Briefly, the samples were labelled A to F and were prepared as described below. The capital letter indicates how it was prepared for the SEM scans after the suspension was prepared. The capital letter indicates any one of: diluted, labelled with the letter (ex: A); further diluted and sonicated, labelled with letter and the number 1 (ex: A1) or freeze-dried (FD), labelled with letter and the number 1 with FD (ex: A1+FD)

Sample A: Chitin milled with water for 3 hours at 670 RPM with 15 balls at a ratio of 0.75:20. This sample corresponds to Example 1 defined above.

Sample B: Chitin milled with water for 9 hours at 670 RPM with 30 balls at a ratio of 1.00:20.

Sample C: Dry chitin milled for 15 minutes at 670 RPM with 5 balls. Chitin milled with water for 3 hours at 670 RPM with 30 balls at a ratio of 1.00:20.

Sample D: Dry chitin milled for 1 hour at 670 RPM with 5 balls. Chitin milled with water for 3 hours at 670 RPM with 30 balls at a ratio of 1.00:20.

Sample E: Dry chitin milled for 3 hours at 670 RPM with 30 balls. Chitin milled with water for 3 hours at 670 RPM with 30 balls at a ratio of 1.25:20.

Sample F: Dry chitin milled for 3 hours at 670 RPM with 30 balls. Chitin milled with water for 3 hours at 670 RPM with 30 balls at a ratio of 1.50:20. This sample corresponds to Example 2 defined above.

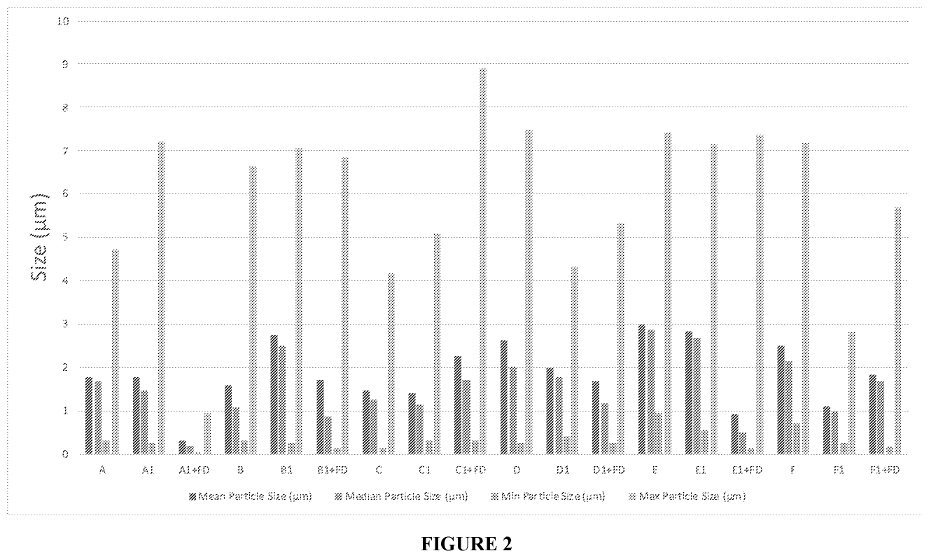

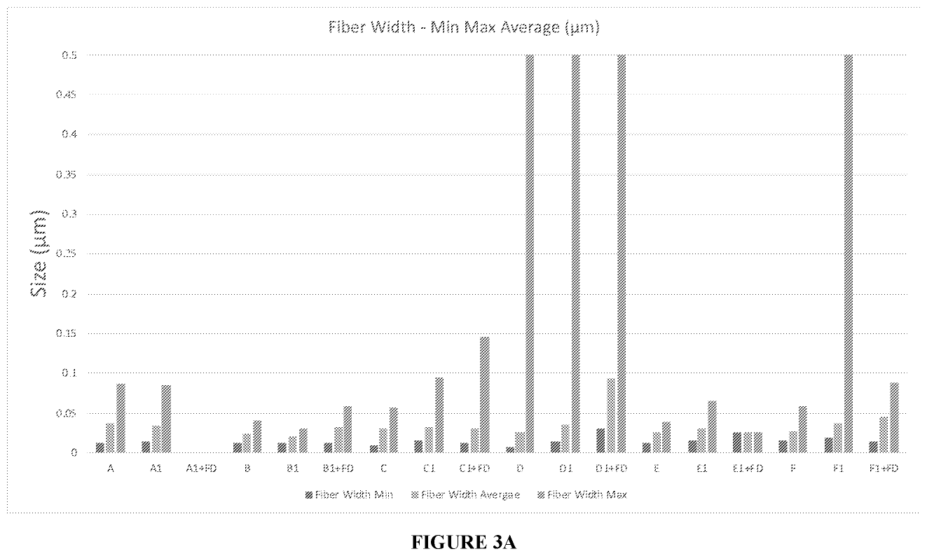

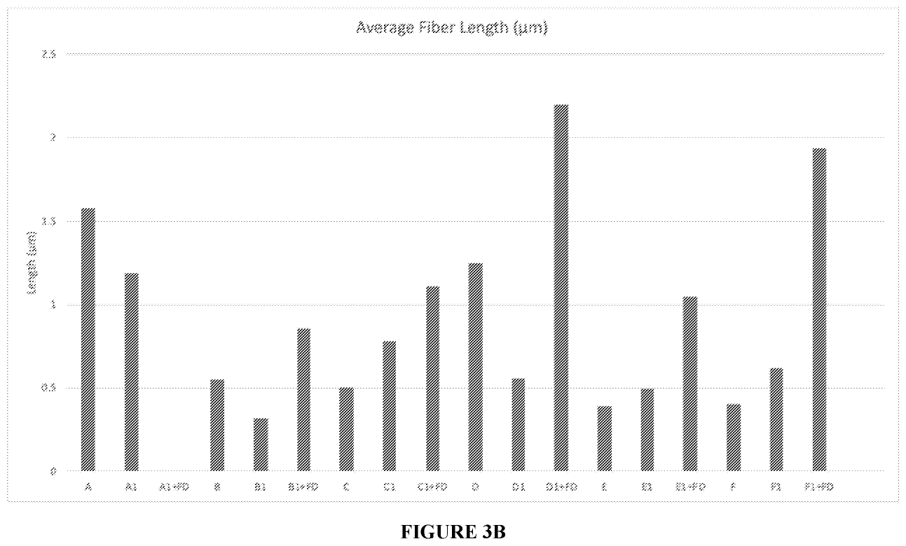

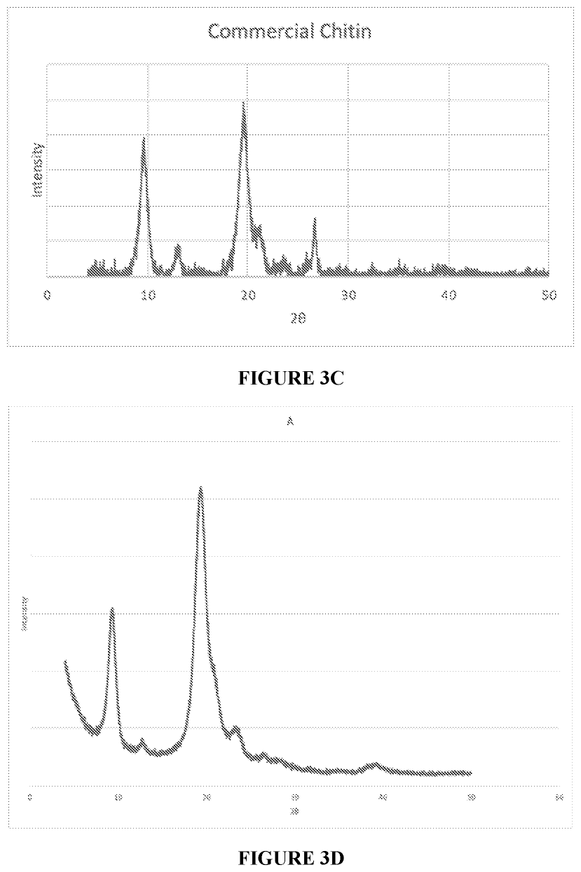

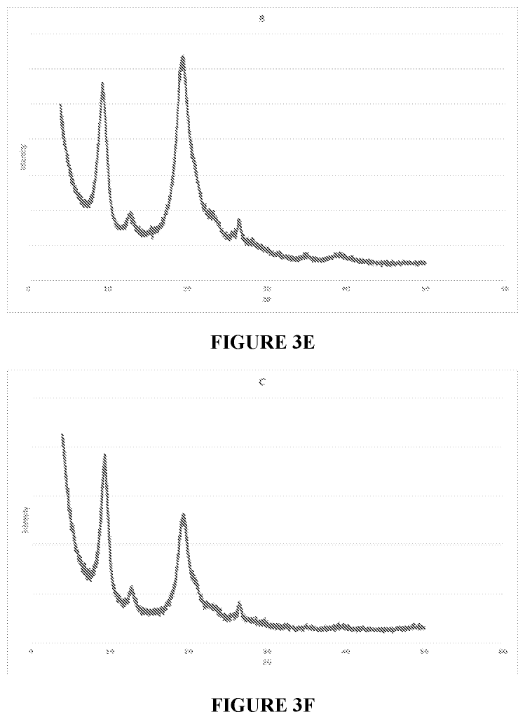

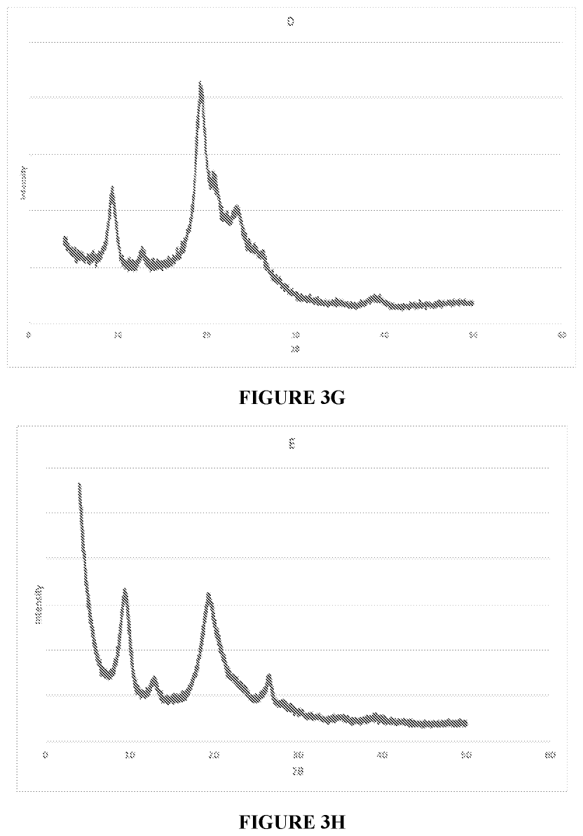

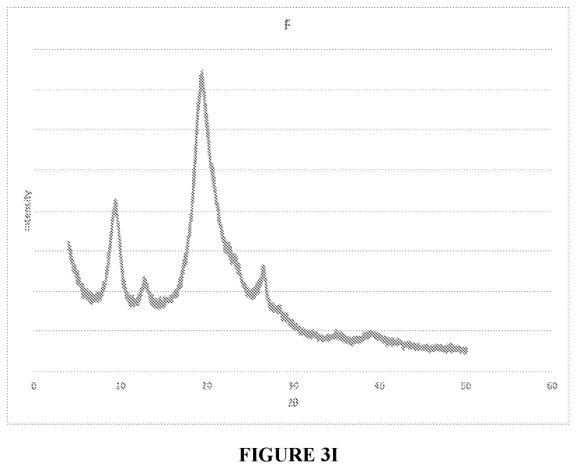

The results are presented in FIGS. 2 and 3. Particularly, in the chitin suspensions, particles varied from 50 nm to 9 μm with averages ranging from 200 nm to 3 μm in size (FIG. 2). Fiber widths varied from 7 nm to 3 μm as agglomerates with an average ranging from 20 nm to 93 nm (FIG. 3A). Fiber length for these samples ranges from 350 nm to 2.5 μm (FIG. 3B).

Table 2 below show the measured viscosities for Samples A-F.

TABLE 2
Measured viscosities of Sample A-F

| Sample | Max Viscosity (mPa · s) |
|---|---|
| A | 11819.13 |
| B | 10849.29 |
| C | 3661.398 |
| D | 11662.38 |
| E | 4077.273 |
| F | 6105.36 |

The results of these experiments show that pre-milling reduces viscosity of the final suspensions. The viscosity was also reduced with an increasing number of balls, and increasing time of mill and increasing speed (i.e., RPM).

On the other hand, it was also possible to obtain suspensions with high viscosities. In one experiment chitin was milled with water for 3 hours at 670 RPM with 10 balls at a ratio of 1.00:20, yielding a viscosity of 40028 mPa·s (data not shown). In another experiment chitin was milled with water for 3 hours at 670 RPM with 20 balls at a ratio of 1.50:20. Yielding a viscosity of 85608 mPa·s (data not shown).

The milling strongly impacted the powder x-ray diffraction (pXRD) patterns. FIG. 3C shows the powder x-ray diffraction of commercial chitin prior to milling. The x-ray pattern demonstrates it to be chitin based on peak positions at about 9.5° and 19.5° 20. This pattern was thus considered to correspond to the signature of chitin in accordance with the present technique (i.e., reference).

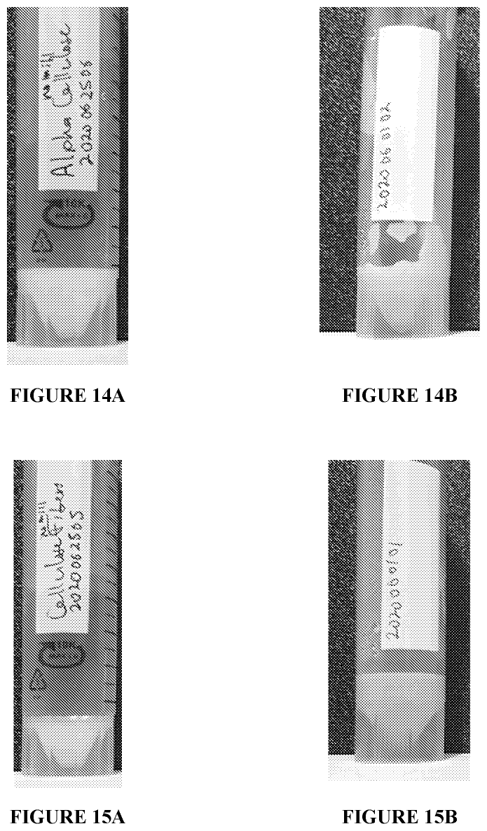

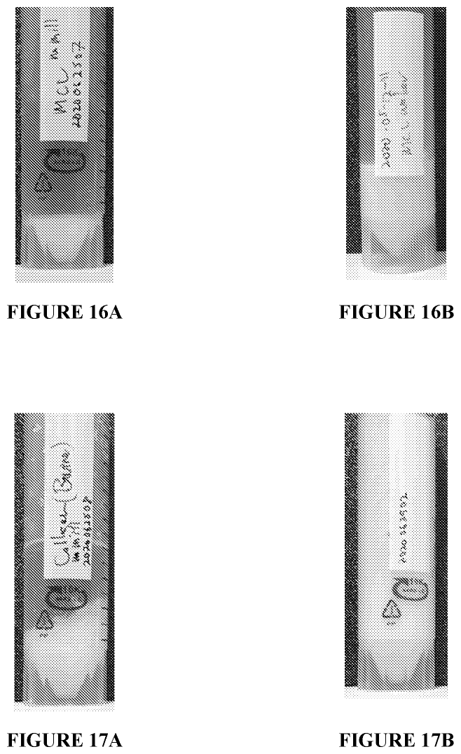

The x-ray patterns for the samples 3A-F for the suspensions (not dried) are shown in FIGS. 3D to 31. Overall, the patterns show that the samples are partially crystalline and partially amorphous, while the x-ray pattern demonstrates it to be chitin based on peak positions at about 9.5° and 19.5° 2θ.

### Example 4: Additional Biopolymer Suspensions

To demonstrate that the present invention is applicable to many different biopolymers, the following insoluble biopolymers were used in processes according to the invention: chitin, chitosan, cellulose (fibres, alpha, microcrystalline), collagen (bovine) and silk.

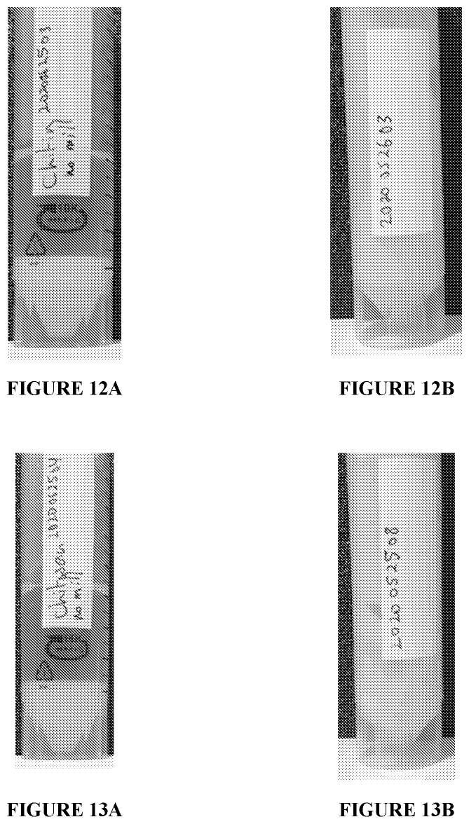

Briefly, biopolymers were milled with water for 3 hours at 670 RPM with 10 balls with diameter of 10 mm at a ratio of 1:20. As depicted in FIGS. 12A to 17B stable homogenous suspensions were successfully obtained for all these biopolymers.

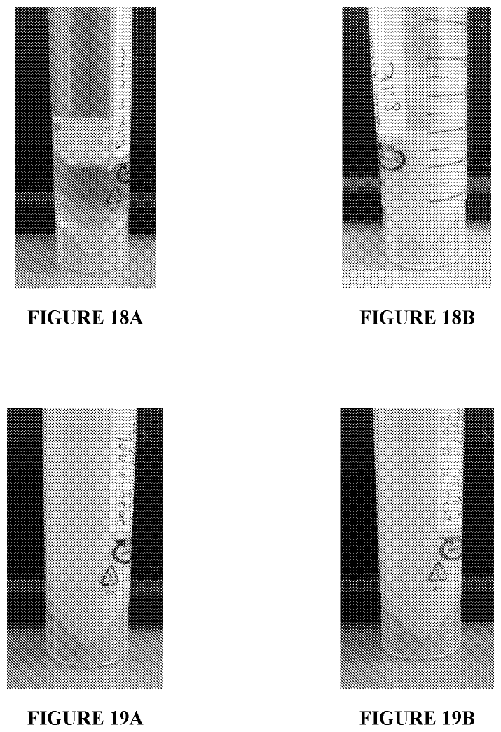

Likewise, silk was pre-milled dry with 40 balls of 10 mm diameter for 3 hours. Silk Fibroin was milled with water for 1 hour at 670 RPM with 40 balls at a ratio of 1:20. As depicted in FIGS. 18A and 18B, a stable homogenous suspension was obtained using that biopolymer.

### Example 5: Combinations of Biopolymers

Chitin and chitosan were milled with water for 3 hours at 670 RPM with 30 balls of 10 mm diameter at a ratio of 0.5:0.5:20 (chitin:chitosan:water). A stable homogenous suspension was obtained (see FIG. 19A).

Chitin and chitosan were milled with water for 3 hours at 670 RPM with 30 balls of 10 mm diameter at a ratio of 0.6:0.4:20 (chitin:chitosan:water). A stable homogenous suspension was obtained (see FIG. 19B).

### Example 6: Additives

### Chitin and Beeswax

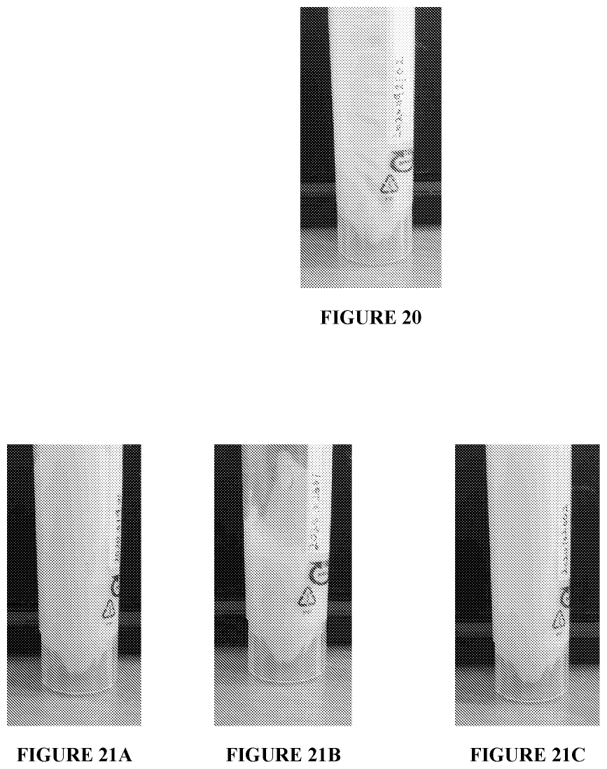

Chitin and beeswax were milled with water for 3 hours at 670 RPM with 50 balls of 10 mm diameter at a ratio of 1:0.25:20 (chitin:beeswax:water). A stable homogenous suspension was obtained (see FIG. 20).

### Chitin and Vegetable Oil

Chitin and vegetable oil were milled with water for 3 hours at 670 RPM with 30 balls of 10 mm diameter at a ratio of 1:20:20 (chitin:vegetable oil:water). A stable homogenous suspension was obtained (see FIG. 21A).

Chitin and vegetable oil milled with water for 3 hours at 670 RPM with 30 balls of 10 mm diameter at a ratio of 1:2:20 (chitin:vegetable oil:water). A stable homogenous suspension was obtained (see FIG. 21B).

Chitin and vegetable oil milled with water for 3 hours at 670 RPM with 30 balls of 10 mm diameter at a ratio of 1:1:20 (chitin:vegetable oil:water). A stable homogenous suspension was obtained (see FIG. 21C).

### Chitin and Soybean Oil

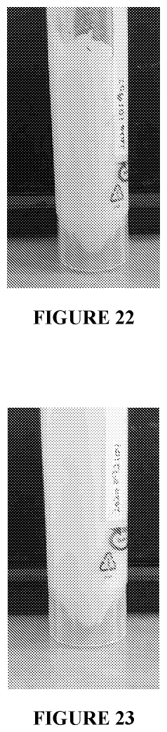

Chitin and soybean oil were milled with water for 3 hours at 670 RPM with 30 balls of 10 mm diameter at a ratio of 1:20:20 (chitin:soybean oil:water). A stable homogenous suspension was obtained (see FIG. 22).

### Example 7: Mixture of Solvents

A combination of two solvents was tested, namely glycerol+water. Briefly, chitin and glycerol were milled with water for 3 hours at 670 RPM with 50 balls of 10 mm diameter at a ratio of 1:0.5:20 (chitin:glycerol:water). A stable homogenous suspension was obtained FIG. 23).

### Example 8: Characterization of Samples by FTIR, SSNMR and PXRD

1) Sample Preparation

The following samples were prepared and used for characterization by FTIR, SSNMR, and PXRD. Milling was carried out using a vertical planetary mill (Tencan XQM-2A™) with 100 mL capacity zirconia jars and 10 mm diameter zirconia balls.

Silk

Fluffy silk was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The silk suspension was generated by milling pre-milled silk in water with a 2.00:20 ratio at 670 RPM with fifty units of 10 mm ball using the 10+1 Alt method, where it is milled for ten minutes followed by a pause for one minute then milling for ten minutes in the opposite direction for a total of 1 hour.

Cellulose

This cellulose suspension was generated by milling cellulose in water with a 1.50:20 ratio at 670 RPM with fifty units of 10 mm ball using the 10+1 Alt method, where it is milled for ten minutes followed by a pause for one minute then milling for ten minutes in the opposite direction for a total of 1 hour.

Collagen

Collagen was pre-milled dry for at 670 RPM with fifty units of 10 mm ball using the 10+1 Alt method, where it is milled for ten minutes followed by a pause for one minute then milling for ten minutes in the opposite direction for a total of 2 hours.

This collagen suspension was generated by milling pre-milled collagen in water with a 2.00:20 ratio at 670 RPM with 50 units of 10 mm ball using the 10+1 Alt method, where it is milled for ten minutes followed by a pause for one minute then milling for ten minutes in the opposite direction for a total of 6 hours.

Alginic Acid

This alginic acid suspension was generated by milling alginic acid in water with a :20 ratio at 670 RPM with 50 units of 10 mm ball using the 10+1 Alt method, where it is milled for ten minutes followed by a pause for one minute then milling for ten minutes in the opposite direction for a total of 3 hours.

Chitin

The chitin suspension was generated by milling chitin in water with a 1.00:20 ratio at 670 RPM with 50 units of 10 mm ball using the 10+1 Alt method, where it is milled for ten minutes followed by a pause for one minute then milling for ten minutes in the opposite direction for a total of 3 hours.

Chitosan

Chitosan was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The chitosan suspension was generated by milling pre-milled chitosan in water with a 0.75:20 ratio at 670 RPM with 30 units of 10 mm ball using the 10+1 Alt method, where it is milled for ten minutes followed by a pause for one minute then milling for ten minutes in the opposite direction for a total of 3 hours.

2) Fourier Transform Infrared Spectroscopy (FTIR) Analysis

Polymer suspensions were prepared as described above. The suspensions were then dried and ground to a powder to perform FTIR spectroscopy. A total of 24 cumulative scans were acquired in the range from 4000 cm-1 to 400 cm-1 with a resolution of 4 cm-1. Graphs of the FTIR spectroscopy analysis are shown in FIGS. 24A to 24F.

Silk

The graph of the FTIR spectroscopy analysis for this sample is shown at FIG. 24A. Relevant peaks are: 3600-2800 cm-1—OH and NH stretch; 1630, 1509 and 1222 cm-1 amide.

Cellulose

The graph of the FTIR spectroscopy analysis for this sample is shown at FIG. 24B. Relevant peaks are: 3300 cm-1—OH stretch; 1624 cm-1—water molecules absorbed in cellulose; ˜1348 cm-1—CH2 and CH3 bends; 1011 cm-1—C—O stretch.

Collagen

The graph of the FTIR spectroscopy analysis for this sample is shown at FIG. 24C. Relevant peaks are: 3262 cm-1—NH stretch; 3039, 2917, 2850 cm-1—C—H stretch; 1625, 1523 cm-1—Amide bends.

Alginic Acid

The graph of the FTIR spectroscopy analysis for this sample is shown at FIG. 24D. Relevant peaks are: 3335 cm-1—OH stretch; 2909 cm-1—C—H stretch; 1711, 1617 cm-1—Carboxylic Acid; 1026 cm-1—C—O stretch.

Chitin

The graph of the FTIR spectroscopy analysis for this sample is shown at FIG. 24E. Relevant peaks are: 3245 cm-1—OH stretch; 3070 cm-1—NH stretch; 2903, 2854 cm-1—Alkenes; 1625, 1540 cm-1—Amide; 1022 cm-1—C—O stretch.

Chitosan

The graph of the FTIR spectroscopy analysis for this sample is shown at FIG. 24F. Relevant peaks are: 3300 cm-1—OH and NH stretch; 2860 cm-1—Alkenes; 1636, 1546 cm-1—Amide; 1020 cm-1—C—O stretch.

3) Solid-State Nuclear Magnetic Resonance Characterization (SSNMR)

Solid-State Nuclear Magnetic Resonance (13C) (SSNMR) was used to determine the composition of the biopolymer suspensions post drying. The suspensions were prepared as described above then dried and ground to a powder.

The data were acquired using a VNMRS 400™ widebore spectrometer operating at 399.9 MHz for ¹H and 100.5 MHz for ¹³C in a 4 mm Varian Chemagnetics™ double-resonance probe. The recycle delay was 4 s. The samples were spun at 13 kHz, with a CP contact time a 2 ms, and 2048 scans were collected for each sample. Graphs of these analysis are shown in FIGS. 25A to 25F.

Silk

The graph of the SSNMR analysis for this sample is shown at FIG. 25A. The following peak shifts demonstrate the corresponding functional group associated to the carbon: 172 ppm—amide; 156 ppm—carbonyl; 62 ppm—C—O; 55 ppm—CH; 49 ppm—CH2; 43 ppm—CH2; 17 ppm—CH3.

Cellulose

The graph of the SSNMR analysis for this sample is shown at FIG. 25B. The following peak shifts demonstrate the corresponding functional group associated to the carbon: 104 ppm—C—O; 82 ppm—O—CH; 75.4 ppm—O—CH; 62.5 ppm—O—CH2.

Collagen

The graph of the SSNMR analysis for this sample is shown at FIG. 25C. The following peak shifts demonstrate the corresponding functional group associated to the carbon: 174 ppm—carbonyl/amide; 71 ppm—C—O; 59 ppm—20 ppm—CH variations; 17 ppm—CH3.

Alginic Acid

The graph of the SSNMR analysis for this sample is shown at FIG. 25D. The following peak shifts demonstrate the corresponding functional group associated to the carbon: 170 ppm—Carbonyl; 103 ppm—C—O; 79 ppm—O—CH; 72 ppm—67 ppm—O—CH.

Chitin

The graph of the SSNMR analysis for this sample is shown at FIG. 25E. The following peak shifts demonstrate the corresponding functional group associated to the carbon: 174 ppm—Carbonyl; 104 ppm—C—O; 83 ppm—55 ppm—O—CH; 23 ppm—CH3.

Chitosan

The graph of the SSNMR analysis for this sample is shown at FIG. 25F. The following peak shifts demonstrate the corresponding functional group associated to the carbon: 174 ppm—Carbonyl; 104 ppm—C—O; 83 ppm—55 ppm—O—CH; 23 ppm—CH3.

4) Power X-Ray Diffraction (PXRD) Characterization

Power X-Ray Diffraction (PXRD) was used to investigate the crystallinity pattern of the biopolymer suspensions post drying. Such patterns can be used as an identification tool for the dried product.

The suspensions were prepared as described above, then dried and ground to a powder. The sample diffractogram was recorded from 4° to 50° with an increment of 0.02 degrees on a zero-background plate using a Bruker D8 ADVANCE™ X-Ray diffractometer equipped with a Cu—Kα (λ=1.54 Å) source. Graphs of the PXRD patterns are shown in FIGS. 26A to 26F.

Silk

The graph of the PXRD pattern for this sample is shown at FIG. 26A. The main peaks are: 2θ=10.71°, 20.64°, 30.45°.

Cellulose

The graph of the PXRD pattern for this sample is shown at FIG. 26B. The main peaks are: 2θ=20.39°, 28.34°, 30.43°, 31.53°, 35.32°.

Collagen

The graph of the PXRD pattern for this sample is shown at FIG. 26C. The main peaks are: 2θ=8.21°, 19.8°, 20.5°, 26.82°, 28.56°, 30.47°, 31.57°, 35.48°.

Alginic Acid

The graph of the PXRD pattern for this sample is shown at FIG. 26D. The main peaks are: 2θ=14.58°, 15.85°, 20.97°, 28.5°, 30.43°.

Chitin

The graph of the PXRD pattern for this sample is shown at FIG. 26E. The main peaks are: 2θ=9.58°, 13.06°, 19.62°, 20.95°, 20.62°, 26.40°, 28.36°, 30.43°, 31.57°, 35.30°.

Chitosan

The graph of the PXRD pattern for this sample is shown at FIG. 26F. The main peaks are: 2θ=13.52°, 20.21°, 28.42°, 30.33°, 31.63°, 35.40°.

### Example 9: Characterization of Samples by Dynamic Light Scattering (DLS)

Dynamic light scattering was used to determine particle size in suspension. The suspensions were prepared as described below.

1) Samples Preparation

Silk

Fluffy silk was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The silk suspension was generated by milling pre-milled silk in water with a 2.00:20 ratio at 670 RPM with fifty units of 10 mm ball using the 10+1 Alt method, where it is milled for ten minutes followed by a pause for one minute then milling for ten minutes in the opposite direction for a total of 1 hour.

Cellulose

The cellulose suspension was generated by milling cellulose in water with a 1.50:20 ratio at 670 RPM with fifty units of 10 mm ball using the 10+1 Alt method, where it is milled for ten minutes followed by a pause for one minute then milling for ten minutes in the opposite direction for a total of 3 hours.

Collagen

Collagen was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The collagen suspension was generated by milling pre-milled collagen in water with a 1.25:20 ratio at 670 RPM with 50 units of 10 mm ball using the 10+1 Alt method, where it is milled for ten minutes followed by a pause for one minute then milling for ten minutes in the opposite direction for a total of 3 hours.

Alginic Acid

The alginic acid suspension was generated by milling alginic acid in water with a 1.00:20 ratio at 670 RPM with 50 units of 10 mm ball using the 10+1 Alt method, where it is milled for ten minutes followed by a pause for one minute then milling for ten minutes in the opposite direction for a total of 3 hours.

Chitosan

Chitosan was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The chitosan suspension was generated by milling pre-milled chitosan in water with a 1.50:20 ratio at 670 RPM with 30 units of 10 mm ball using the 10+1 Alt method, where it is milled for ten minutes followed by a pause for one minute then milling for ten minutes in the opposite direction for a total of 2 hours.

2) DLS Measurements

Samples prepared as described above were diluted in water, using one drop of sample in 15 mL of water where the dilution is not turbid. Measurements were completed in triplicate for a duration of 2 minutes each time. The temperature was maintained at 25° C., Viscosity (cP): 0.8900, Refractive Index: 1.3310, and Scattering Angle 900.

The values determined using DLS represent swollen polymer particles, as compared to dry polymer particles with SEM.

3) Results

Table 3 below summarize the results of the measurements of the particle size in each of the samples:

TABLE 3
DLS measurements of prepared suspensions

| | Average | Mean | Mean | Mean |
|---|---|---|---|---|
| | Effective | Diam. by | Diam. by | Diam. by |
| | Diameter 50% | Intensity | Volume | Number |
| Sample | (nm) | (nm) | (nm) | (nm) |
| Silk | 900.96 | 1288.84 | 998.67 | 335.39 |
| 2021030502 | | | | |
| Cellulose | 1092.22 | 1756.43 | 1092.66 | 414.15 |
| 2021040802 | | | | |
| Collagen | 2506.07 | 4830.67 | 4471.28 | 848.98 |
| 2021030803 | | | | |
| Alginic Acid | 844.12 | 1129.44 | 958.51 | 471.23 |
| 2021031910 | | | | |
| Chitosan | 659.57 | 1289.85 | 1132.48 | 177.135 |
| 2021030801 | | | | |

### Example 10: Characterization of Samples by Light Transmittance

Transmittance demonstrates the ability for light to pass through a substance. This measure can indicate the opacity of a suspension and spectra can be compared for distinguishing various nano-biopolymer suspensions/solutions.

1) Samples Preparation

Silk

Fluffy silk was pre-milled dry for at 670 RPM with fifty units of 10 mm ball using the 10+1 Alt method, where it is milled for ten minutes followed by a pause for one minute then milling for ten minutes in the opposite direction for a total of 1 hour.

The silk suspension was generated by milling pre-milled silk in water with a 1.50:20 ratio at 670 RPM with fifty units of 10 mm ball using the 10+1 Alt method, where it is milled for ten minutes followed by a pause for one minute then milling for ten minutes in the opposite direction for a total of 1 hour.

Cellulose

The cellulose suspension was generated by milling cellulose in water with a 2.00:20 ratio at 670 RPM with fifty units of 10 mm ball using the 10+1 Alt method, where it is milled for ten minutes followed by a pause for one minute then milling for ten minutes in the opposite direction for a total of 3 hours.

Collagen

Collagen was pre-milled dry for at 670 RPM with fifty units of 10 mm ball using the 10+1 Alt method, where it is milled for ten minutes followed by a pause for one minute then milling for ten minutes in the opposite direction for a total of 2 hours.

The collagen suspension was generated by milling pre-milled collagen in water with a 1.00:20 ratio at 670 RPM with 50 units of 10 mm ball using the 10+1 Alt method, where it is milled for ten minutes followed by a pause for one minute then milling for ten minutes in the opposite direction for a total of 3 hours.

Alginic Acid

The alginic acid suspension was generated by milling alginic acid in water with a 1.00:20 ratio at 670 RPM with 50 units of 10 mm ball using the 10+1 Alt method, where it is milled for ten minutes followed by a pause for one minute then milling for ten minutes in the opposite direction for a total of 3 hours.

Chitin

The chitin suspension was generated by milling chitin in water with a 0.60:20 ratio at 670 RPM with 50 units of 10 mm ball using the 10+1 Alt method, where it is milled for ten minutes followed by a pause for one minute then milling for ten minutes in the opposite direction for a total of 3 hours.

Chitosan

Chitosan was pre-milled dry for at 670 RPM with thirty units of 10 mm ball using the 10+1 Alt method, where it is milled for ten minutes followed by a pause for one minute then milling for ten minutes in the opposite direction for a total of 1 hour.

The chitosan suspension was generated by milling pre-milled chitosan in water with a 1.00:20 ratio at 670 RPM with 30 units of 10 mm ball using the 10+1 Alt method, where it is milled for ten minutes followed by a pause for one minute then milling for ten minutes in the opposite direction for a total of 1 hour.

2) Transmittance Measurements

The above samples were measured as is in a quartz cuvette for % transmittance mode from 200 nm to 800 nm on a thermo Scientific Evolution™ 260 bio. Additionally, absorbance in the range of 400-320 nm is the UV-A range and in the range of 320-280 nm is the UV-B range for sunscreen. All suspensions show absorbance from 290 nm to 800 nm.

Graphs of the transmittance spectra are shown in FIGS. 27A to 27F for silk (FIG. 27A), for cellulose (FIG. 27B), for collagen (FIG. 27C), for alginic acid (FIG. 27D), for chitin (FIG. 27E) and chitosan (FIG. 27F). Although not shown, the percentage of transmittance increased when the sample was diluted.

### Example 11: Characterization of Samples by Scanning Electron Microscope (SEM)

1) Sample Preparation

Silk

Fluffy silk was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The silk suspension was generated by milling pre-milled silk in water with a 1.00:20 ratio at 670 RPM with fifty units of 10 mm ball using the 10+1 Alt method, where it is milled for ten minutes followed by a pause for one minute then milling for ten minutes in the opposite direction for a total of 15 minutes or 1 hour or 3 hours.

Cellulose

The cellulose suspension was generated by milling cellulose in water with a 1.00:20 ratio at 670 RPM with fifty units of 10 mm ball using the 10+1 Alt method, where it is milled for ten minutes followed by a pause for one minute then milling for ten minutes in the opposite direction for a total of 15 minutes or 1 hour or 3 hours.

Alginic Acid

The alginic acid suspension was generated by milling alginic acid in water with a 1.00:20 ratio at 670 RPM with 50 units of 10 mm ball using the 10+1 Alt method, where it is milled for ten minutes followed by a pause for one minute then milling for ten minutes in the opposite direction for a total of 15 minutes or 1 hour or 3 hours.

Chitin

The chitin suspension was generated by milling chitin in water with a 1.00:20 ratio at 670 RPM with 50 units of 10 mm ball using the 10+1 Alt method, where it is milled for ten minutes followed by a pause for one minute then milling for ten minutes in the opposite direction for a total of 15 minutes, or 1 hour or 3 hours.

Chitosan

Chitosan was pre-milled dry for at 670 RPM with thirty units of 10 mm ball using the 10+1 Alt method, where it is milled for ten minutes followed by a pause for one minute then milling for ten minutes in the opposite direction for a total of 3 hours.

The chitosan suspension was generated by milling pre-milled chitosan in water with a 1.00:20 ratio at 670 RPM with 50 units of 10 mm ball using the 10+1 Alt method, where it is milled for ten minutes followed by a pause for one minute then milling for ten minutes in the opposite direction for a total of 15 minutes or 1 hour or 3 hours.

2) SEM Imaging

Polymer suspensions were prepared as described above. Then sample suspensions were diluted, one drop into 5 mL of water. One drop of the dilution was added to an SEM stub then coated with platinum prior to measurement.

SEM pictures are shown in FIGS. 28A to 32G. Table 4 below summarizes the observed properties of the samples milled in water for either 15 mins, 1 hour or 3 hours.

TABLE 4
Properties of samples assessed by SEM

| | | | Range of | | | |
|---|---|---|---|---|---|---|
| | | | particles | Average | Median | |
| | | | size (nm) | size (nm) | size (nm) | Dominant shape |
| Alginic acid | 15 | min | 11-149 | 48 | 43 | agglomerated spheres + |
| | | | | | | presence of fibers |
| | 1 | hour | 10-202 | 72 | 64 | agglomerated spheres + |
| | | | | | | presence of platelets |
| | 3 | hours | 14-174 | 44 | 36 | agglomerated spheres |
| Cellulose | 15 | min | 34-115 | 59 | 56 | agglomerated spheres |
| | 1 | hour | 15-195 | 55 | 40 | agglomerated spheres |
| | 3 | hours | 23-200 | 71 | 63 | agglomerated spheres |
| Chitin | 15 | min | 3-188 | 78 | 74 | agglomerated spheres |
| | 1 | hour | 10-215 | 69 | 60 | agglomerated spheres |
| | 3 | hours | 23-110 | 52 | 51 | agglomerated spheres + |
| | | | | | | agglomerated fibers |
| Chitosan | 15 | min | 22-204 | 85 | 81 | agglomerated spheres |
| | 1 | hour | 13-516 | 103 | 80 | agglomerated spheres |
| | 3 | hours | 24-431 | 110 | 92 | agglomerated spheres |
| Silk | 15 | min | 26-422 | 107 | 85 | agglomerated spheres + |
| | | | | | | agglomerated fibers |
| | 1 | hour | 20-153 | 46 | 43 | agglomerated spheres |
| | 3 | hours | 57-404 | 157 | 137 | agglomerated spheres + |
| | | | | | | presence of platelets |

### Example 12: Biopolymer Sweep Suspension Tests

To further investigate biopolymer/material suspensions produced via milling, a sweep of milled samples and their non-milled version were compared for a visual confirmation of differentiation. Chitin, chitosan, cellulose, collagen, pectin, gelatin, and beeswax were studied for suspension-ability.

Fine powdered biopolymer samples were added to water in a 1:20 ratio. Mixtures were milled in accordance to the 10+1 Alt method, where it is milled for ten minutes followed by a pause for one minute then milling for ten minutes in the opposite direction for a total of 3 hours.

Visual assessments prior and after milling were assessed and compared. Chitin, chitosan, cellulose, collagen, alginic acid were all found to not dissolve in water prior to the milling process, whereas they were successfully suspended after the milling. However, pectin, gelatin, lignin, guar gum and xantham gum showed some or complete solubility and could, in some cases, be milled to dissolve further. Beeswax does not dissolve nor suspend with milling; it floats at the surface of the water. Agarose does not dissolve in water and milling creates a thick solid gel. These observations are summarized in Table 5.

TABLE 5
Suspension of biopolymer prior and after milling

| Biopolymer | Simple Mixing | Milling | Conclusion |
|---|---|---|---|
| Chitin (Sigma) | Mixing the biopolymer | Milling the mixture | Suspendable |
| | with water does not | under standard | |
| | show any solubility or | conditions produces a | |
| | suspension | suspension | |
| Chitosan | Mixing the biopolymer | Milling the mixture | Suspendable |
| (Sigma) | with water does not | under standard | |
| | show any solubility or | conditions produces a | |
| | suspension | suspension | |
| Alpha | Mixing the biopolymer | Milling the mixture | Suspendable |
| Cellulose | with water does not | under standard | |
| (Sigma) | show any solubility or | conditions produces a | |
| | suspension | suspension | |
| Cellulose | Mixing the biopolymer | Milling the mixture | Suspendable |
| Fibers | with water does not | under standard | |
| (Sigma) | show any solubility or | conditions produces a | |
| | suspension | suspension | |
| Microcrystalline | Mixing the biopolymer | Milling the mixture | Suspendable |
| Cellulose | with water does not | under standard | |
| (Ingredient | show any solubility or | conditions produces a | |
| Depot) | suspension | suspension | |
| Collagen | Mixing the biopolymer | Milling the mixture | Suspendable |
| (Sigma) | with water does not | under standard | |
| | show any solubility or | conditions produces a | |
| | suspension | suspension | |
| Silk | Mixing the biopolymer | Milling the mixture | Suspendable |
| | with water does not | under standard | |
| | show any solubility or | conditions produces a | |
| | suspension | suspension | |
| Alginic acid | Mixing the biopolymer | Milling the mixture | Suspendable |
| | with water does not | under standard | |
| | show any solubility or | conditions produces a | |
| | suspension | suspension | |
| Pectin (Sigma) | Mixing the biopolymer | Milling the biopolymer | Dissolves |
| | with water shows | with water under | |
| | solubility | standard conditions | |
| | | also shows solubility | |
| Pectin | Mixing the biopolymer | Milling the mixture | Dissolves |
| (Bernardin) | with water shows | under standard | |
| | solubility | conditions appears to | |
| | | dissolve it | |
| Gelatin | Mixing the biopolymer | Milling the mixture | Dissolves |
| (Sigma) | with water shows | under standard | |
| | solubility | conditions appears to | |
| | | dissolve it (most likely | |
| | | from heat generation | |
| | | during milling) | |
| Gelatin (Knox) | Mixing the biopolymer | Milling the mixture | Dissolves |
| | with water shows some | under standard | |
| | solubility | conditions appears to | |
| | | dissolve it (most likely | |
| | | from heat generation | |
| | | during milling) | |
| Kraft Lignin | Mixing the biopolymer | N/A | Dissolves |
| | with water does not | | |
| | show any solubility or | | |
| | suspension | | |
| Guar gum | Mixing the biopolymer | N/A | Dissolves/Swells |
| | with water shows | | |
| | solubility | | |
| Xanthan gum | Mixing the biopolymer | N/A | Dissolves/Swells |
| | with water shows | | |
| | solubility | | |
| Beeswax | Mixing the biopolymer | Milling the mixture | Not |
| (Sigma) | with water does not | under standard | Suspendable |
| | show any solubility or | conditions does not | |
| | suspension | produce a suspension. | |
| ABLE beeswax | Mixing the biopolymer | Milling the mixture | Not |
| | with water does not | under standard | Suspendable |
| | show any solubility or | conditions does not | |
| | suspension | produce a suspension. | |
| Agarose | Mixing the biopolymer | Milling the mixture | Suspendable/ |
| | with water does not | under standard | Dissolvable |
| | show any solubility or | conditions produces a | |
| | suspension | thick suspension/gel. | |

### Example 13: Rheological Behavior

Rheological data of biopolymer suspensions may be useful to demonstrate that sheer thinning is observed. It may also give an example of the viscosity achieved with a specific formulation. Accordingly, rheological behavior of chitin, chitosan, cellulose, collagen, silk, and alginic acid various polymer suspensions were investigated as well as blends consisting of chitin-silk-collagen, chitin-mineral oil and chitin-beeswax.

1) Samples Preparation

Silk

Fluffy silk was pre-milled dry for at 670 RPM with fifty units of 10 mm ball using the 10+1 Alt method, where it is milled for ten minutes followed by a pause for one minute then milling for ten minutes in the opposite direction for a total of 6 hours.

The silk suspension was generated by milling pre-milled silk in water with a 2.00:20 ratio at 670 RPM with fifty units of 10 mm ball using the 10+1 Alt method, where it is milled for ten minutes followed by a pause for one minute then milling for ten minutes in the opposite direction for a total of 6 hours.

Cellulose

The cellulose suspension was generated by milling cellulose in water with a 1.50:20 ratio at 670 RPM with fifty units of 10 mm ball using the 10+1 Alt method, where it is milled for ten minutes followed by a pause for one minute then milling for ten minutes in the opposite direction for a total of 3 hours.

Collagen 1.25

Collagen was pre-milled dry for at 670 RPM with fifty units of 10 mm ball using the 10+1 Alt method, where it is milled for ten minutes followed by a pause for one minute then milling for ten minutes in the opposite direction for a total of 3 hours.

The collagen suspension was generated by milling pre-milled collagen in water with a 1.25:20 ratio at 670 RPM with 50 units of 10 mm ball using the 10+1 Alt method, where it is milled for ten minutes followed by a pause for one minute then milling for ten minutes in the opposite direction for a total of 3 hours.

Collagen 1.50

Collagen was pre-milled dry for at 670 RPM with fifty units of 10 mm ball using the 10+1 Alt method, where it is milled for ten minutes followed by a pause for one minute then milling for ten minutes in the opposite direction for a total of 2 hours.

The collagen suspension was generated by milling pre-milled collagen in water with a 1.50:20 ratio at 670 RPM with 50 units of 10 mm ball using the 10+1 Alt method, where it is milled for ten minutes followed by a pause for one minute then milling for ten minutes in the opposite direction for a total of 1 hour.

Alginic Acid

The alginic acid suspension was generated by milling alginic acid in water with a 1.00:20 ratio at 670 RPM with 50 units of 10 mm ball using the 10+1 Alt method, where it is milled for ten minutes followed by a pause for one minute then milling for ten minutes in the opposite direction for a total of 3 hours.

Chitin

The chitin suspension was generated by milling chitin in water with a 1.00:20 ratio at 670 RPM with 50 units of 10 mm ball using the 10+1 Alt method, where it is milled for ten minutes followed by a pause for one minute then milling for ten minutes in the opposite direction for a total of 3 hours.

Chitosan

Chitosan was pre-milled dry for at 670 RPM with 30 units of 10 mm ball using the 10+1 Alt method, where it is milled for ten minutes followed by a pause for one minute then milling for ten minutes in the opposite direction for a total of 1 hour.

The chitosan suspension was generated by milling pre-milled chitosan in water with a 1.50:20 ratio at 670 RPM with 50 units of 10 mm ball using the 10+1 Alt method, where it is milled for ten minutes followed by a pause for one minute then milling for ten minutes in the opposite direction for a total of 1 hour.

Chitin Mineral Oil

The chitin suspension was generated by milling chitin with mineral oil in water with a 1.00:0.50:20 ratio at 670 RPM with 50 units of 10 mm ball using the 10+1 Alt method, where it is milled for ten minutes followed by a pause for one minute then milling for ten minutes in the opposite direction for a total of 3 hours.

Chitin Beeswax

The chitin suspension was generated by milling chitin in water with a 0.90:20 ratio at 670 RPM with 50 units of 10 mm ball using the 10+1 Alt method, where it is milled for ten minutes followed by a pause for one minute then milling for ten minutes in the opposite direction for a total of 3 hours. Beeswax was added to a ratio of 0.50:0.90:20 and milled for 3 hours under the same conditions.

Chitin Collagen Silk

Collagen was pre-milled dry for at 670 RPM with fifty units of 10 mm ball using the 10+1 Alt method, where it is milled for ten minutes followed by a pause for one minute then milling for ten minutes in the opposite direction for a total of 2 hours.

Fluffy silk was pre-milled dry for at 670 RPM with fifty units of 10 mm ball using the 10+1 Alt method, where it is milled for ten minutes followed by a pause for one minute then milling for ten minutes in the opposite direction for a total of 6 hours.

The chitin collagen silk suspension was generated by milling chitin, collagen and silk in water with a 0.70:0.15:0.15:20 ratio at 670 RPM with 50 units of 10 mm ball using the 10+1 Alt method, where it is milled for ten minutes followed by a pause for one minute then milling for ten minutes in the opposite direction for a total of 3 hours.

2) Results

FIG. 33 shows the rheology polymer sweep of each the polymer suspensions as well as for blends thereof.

### Example 14: Rheology Chitin Pre-Mill Effect

The rheological effect of chitin suspensions was compared with ratio and pre-mill effects. Chitin suspensions were prepared under the same conditions for ratios of 0.60, 0.80, 1.00 and 2.00, where the chitin was used as-is or it was pre-milled to reduce particle size.

No pre-milling: The chitin suspension was generated by milling chitin in water with a 0.60:20 (or 0.8:20, or 1.00:20 or 2.00:20) ratio at 670 RPM with 50 units of 10 mm ball using the 10+1 Alt method, where it is milled for ten minutes followed by a pause for one minute then milling for ten minutes in the opposite direction for a total of 3 hours.

With pre-milling: Chitin was pre-milled dry for at 670 RPM with 50 units of 10 mm ball using the 10+1 Alt method, where it is milled for ten minutes followed by a pause for one minute then milling for ten minutes in the opposite direction for a total of 3 hours. The chitin suspension was generated by milling pre-milled chitin in water with a 0.60:20 (or 0.8:20, or 1.00:20 or 2.00:20) ratio at 670 RPM with 50 units of 10 mm ball using the 10+1 Alt method, where it is milled for ten minutes followed by a pause for one minute then milling for ten minutes in the opposite direction for a total of 3 hours.

As shown in FIG. 34, the overall comparison shows that pre-milling reduces final viscosity regardless of the chitin ratio used. Therefore, these results show that the viscosity can be reduced by more than half by a pre-milling step. This is very advantageous since it allows for more material to be included, if required, without affecting the final viscosity of the biopolymer suspension.

### Example 15: 1 H NMR of N-Acetyl Glucosamine

In general, the chitin suspensions described herein are composed solely of chitin and water with the extent of chitin degradation predicted to reach water-soluble forms of the polymer. ¹HNMR spectroscopy was conducted in order to gain insight into the types of species of biopolymers present in the present chitin formulations. Preliminary results indicate the presence of water-soluble components with signatures partially matching predicted spectrums for monomer and dimer forms of chitin.

1) Samples Preparation

Two chitin suspensions were generated as follows. Chitin was milled in water with a 0.90:20 ratio at 670 RPM with ninety units of 10 mm ball using the 10+1Alt method, where it is milled for ten minutes followed by a pause for one minute then milling for ten minutes in the opposite direction for a total of 12 hours. The chitin suspension was then filtered under vacuum using a 3 μm Whatman™ filter in order to capture water soluble components of the formulations.

Samples were then submitted to ¹H NMR spectroscopy, where the filtrates were lyophilized and resulting solids were resuspended with D₂O prior to analyses. A N-Acetyl Glucosamine standard was also analyzed simultaneously.

2) Results

Predictive Spectrum

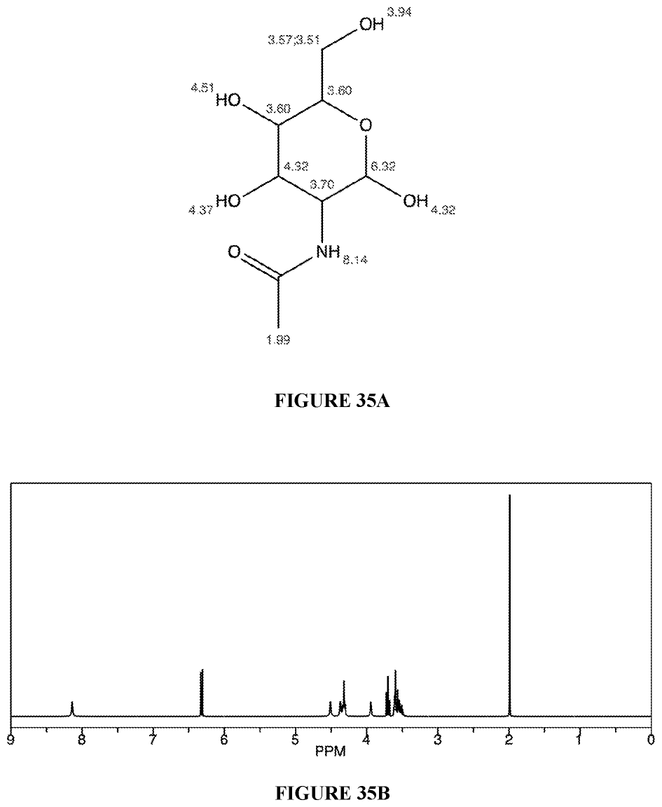

The predictive spectrum for N-Acetyl Glucosamine (NAG; FIG. 35A) ¹HNMR was generated using the ChemDraw™ software (V 16.0.1.49) as shown in FIG. 35B.

Comparison of NAG Standard to Chitin Suspension Water-Soluble Filtrate

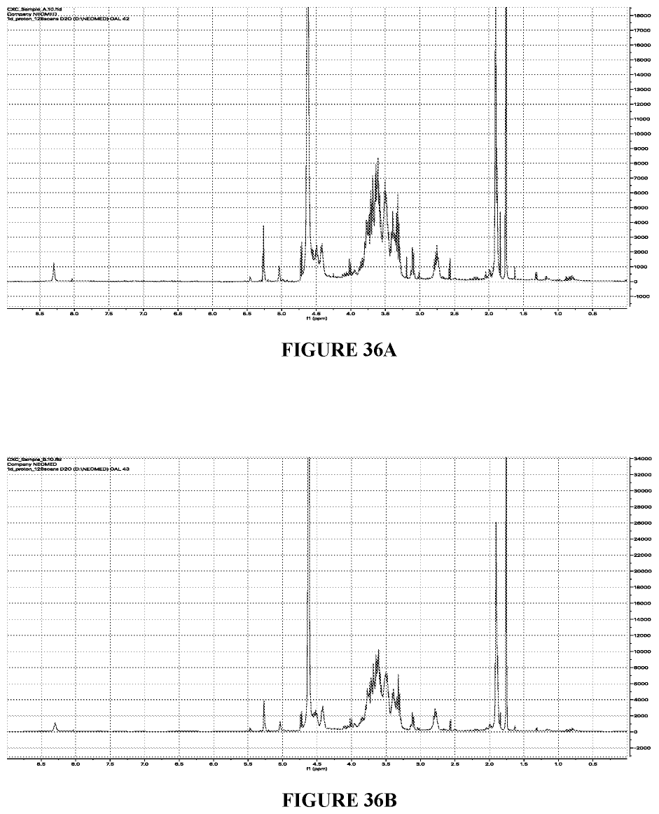

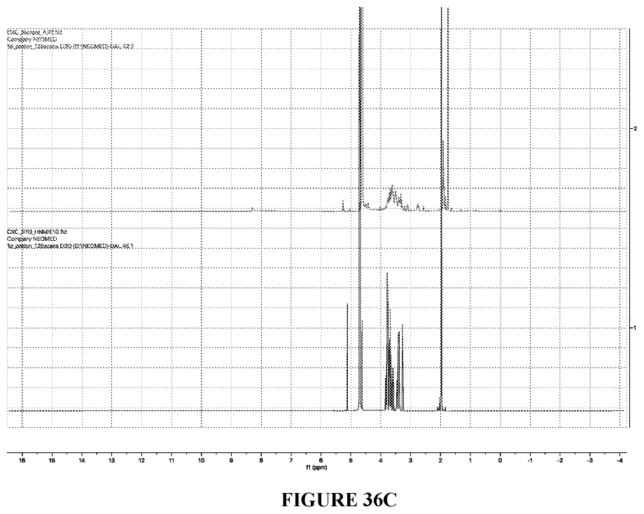

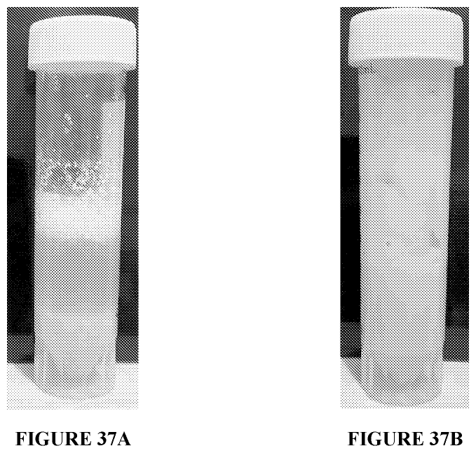

Two chitin suspensions (#1 and #2) were generated as follows. Briefly, chitin was milled in water with a 0.90:20 ratio at 670 RPM with ninety units of ten mm ball using the 10+1Alt method, where it is milled for ten minutes followed by a pause for one minute then milling for ten minutes in the opposite direction for a total of 12 hours. The chitin suspensions was then filtered under vacuum using a 3 μm Whatman™ filter in order to capture water soluble components of the formulations. The two suspensions were next analyzed via ¹HNMR spectrometry. Similar overall spectra were noted for both replicates indicating consistent generation of water-soluble components through the methods described (FIG. 36A and FIG. 36B).

¹HNMR signatures for chitin suspension #2 was subsequently compared to the ¹HNMR spectrum generated for an N-Acetyl Glucosamine standard. As shown in FIG. 36C an overall concordance was found between these spectra providing preliminary evidence for the presence of chitin monomer species and other water-soluble chitin components in the chitin suspensions in accordance with the present invention.

### Example 16: N-Acetyl Glucosamine Doped Chitin Suspension

Since the chitin suspensions in accordance with the present invention consist uniquely of long chains of N-Acetyl Glucosamine, the stability of the suspensions was investigated with the addition of N-Acetyl Glucosamine monomers.

A chitin suspension was generated by milling chitin and N-Acetyl Glucosamine (NAG) in water with a ratio of a) 0.80:0.04:20 (i.e., 5% w/w NAG) or b) 0.80:0.08:20 (i.e., 10% w/w NAG) at 670 RPM with fifty units of 10 mm ball using the 10+1 Alt method, where it is milled for ten minutes followed by a pause for one minute then milling for ten minutes in the opposite direction for a total of 3 hours.

Although not shown, the resulting chitin-NAG-water suspensions were homogeneous and stable. These results demonstrate that N-Acetyl Glucosamine monomers can be incorporated into the chitin suspensions as additives, thereby showing great promise for the addition of established anti-aging agents such as NAG to this formulations of the present invention.

### Example 17: Makeup Color Tests

Studies were conducted to test the ability of the biopolymer suspensions of the invention to carry colored additives and powders such as mica powders.

A chitin suspension was generated by milling chitin in water with a 0.80:20 ratio at 670 RPM with thirty units of 10 mm ball using the 10+1 Alt method, where it is milled for ten minutes followed by a pause for one minute then milling for ten minutes in the opposite direction for a total of 3 hours.

Commercially-available mica powders of various colors (e.g., bronze, mustard, cobalt, teal, mauve, red) were then added to chitin suspensions individually and mixed manually with a spatula. Mica quantities ranging from 10 mg to 100 mg in 3 ml of suspensions were prepared.

As an exemplary test, mica powders (100 mg) of various colors were homogenously suspended in the chitin preparations and then applied to the skin.

Although not shown, the preparations were found to dry evenly and were smooth to the touch without flaking off. The intensity of color saturation was proportional to the quantity of mica introduced to the suspensions. Colored suspensions were easily washed off, by rubbing with water, without leaving colored residues on the users' skin.

These results indicate that mica is able to suspend properly in chitin suspensions, confirming the uses of the biopolymer formulations of the invention for make-up-related cosmetics applications.

### Example 18: Oil and Wax Biopolymer Suspensions

Biopolymer suspensions in accordance with the present invention were investigated for their ability to remain homogeneous in the presence of additives such as oils and waxes.

1) Samples Preparation

Chitin-Mineral Oil: A chitin suspension was generated by milling chitin and mineral oil in water with a 1.00:0.50:20 ratio at 670 RPM with fifty units of 10 mm ball using the 10+1 Alt method, where it is milled for ten minutes followed by a pause for one minute then milling for ten minutes in the opposite direction for a total of 3 hours.

Chitin-Beeswax: A chitin suspension was generated by milling chitin in water with a 0.90:20 ratio at 670 RPM with fifty units of 10 mm ball using the 10+1 Alt method, where it is milled for ten minutes followed by a pause for one minute then milling for ten minutes in the opposite direction for a total of 3 hours. Beeswax was added to the chitin suspension then milled for another 3 hours, to yield a final chitin:beeswax:water ratio of 0.90:0.50:20.

Chitosan-Additive: Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The chitosan suspension was generated by milling chitosan in water with a 1.20:20 ratio at 670 RPM with fifty units of 10 mm ball using the 10+1 Alt method, where it is milled for ten minutes followed by a pause for one minute then milling for ten minutes in the opposite direction for a total of 2 hours. Beeswax or mineral oil was added to the chitosan suspension then milled for another 2 hours, to yield a final chitosan:beeswax:water or chitosan:mineral oil:water ratio of 1.20:0.50:20.

Cellulose-Additive: A cellulose suspension was generated by milling cellulose in water with a 1.00:20 ratio at 670 RPM with fifty units of 10 mm ball using the 10+1 Alt method, where it is milled for ten minutes followed by a pause for one minute then milling for ten minutes in the opposite direction for a total of 1 hour. Beeswax or mineral oil was added to the cellulose suspension then milled for another 1 hour, to yield a final cellulose:beeswax:water or cellulose:mineral oil:water ratio of 1.00:0.50:20.

Alginic Acid-Additive: An alginic acid suspension was generated by milling alginic acid in water with a 2.00:20 ratio at 670 RPM with fifty units of 10 mm ball using the 10+1 Alt method, where it is milled for ten minutes followed by a pause for one minute then milling for ten minutes in the opposite direction for a total of 3 hours. Beeswax or mineral oil was added to the alginic acid suspension then milled for another 3 hours, to yield a final alginic acid:beeswax:water or alginic acid:mineral oil:water ratio of 2.00:0.50:20.

Collagen-Additive: Collagen was pre-milled dry at 670 RPM with fifty units of 10 mm ball using the 10+1 Alt method, where it is milled for ten minutes followed by a pause for one minute then milling for ten minutes in the opposite direction for a total of 3 hours. The collagen suspension was generated by milling collagen in water with a 1.00:20 ratio at 670 RPM with fifty units of 10 mm ball using the 10+1 Alt method, where it is milled for ten minutes followed by a pause for one minute then milling for ten minutes in the opposite direction for a total of 3 hours. Beeswax or mineral oil was added to the collagen suspension then milled for another 3 hours, to yield a final collagen:beeswax:water or collagen:mineral oil:water ratio of 1.00:0.50:20.

Silk-Additive: Silk was pre-milled dry at 670 RPM with fifty units of 10 mm ball using the 10+1 Alt method, where it is milled for ten minutes followed by a pause for one minute then milling for ten minutes in the opposite direction for a total of 3 hours. The silk suspension was generated by milling silk in water with a 1.00:20 ratio at 670 RPM with fifty units of 10 mm ball using the 10+1 Alt method, where it is milled for ten minutes followed by a pause for one minute then milling for ten minutes in the opposite direction for a total of 6 hours. Beeswax or mineral oil was added to the silk suspension then milled for another 3 hours, to yield a final silk:beeswax:water or silk:mineral oil:water ratio of 1.00:0.50:20.

2) Results

The resulting biopolymer-additive-water suspensions were stable and homogeneous for chitin blends, chitosan blends cellulose blends, alginic acid blends, collagen blends, and silk blends. All resulting blends provided smooth application on the skin (data not shown). These results confirm that the biopolymer suspensions in accordance with the present invention can successfully incorporate additives.

### Example 19: Preparation of a Ginseng Suspension

A ginseng suspension was generated by milling ginseng powder in water with a 1.00:20 ratio at 670 RPM with fifty units of 10 mm ball using the 10+1 Alt method, where it is milled for ten minutes followed by a pause for one minute then milling for ten minutes in the opposite direction for a total of 1 hour.

As depicted in (Erreur ! Source du renvoi introuvable. and 2), ginseng powder was not soluble in water but milling resulted in a stable and homogeneous suspension. It was also possible to decrease the viscosity of the milled suspension by increasing the shear rate, as detailed in Table 6.

TABLE 6
Viscosity of suspended ginseng

| Shear Rate (s−1) | Viscosity (mPas) |
|---|---|
| 0.14 | 21000 |
| 0.28 | 10080 |
| 0.56 | 5375 |
| 1.4 | 2683 |
| 2.24 | 1729 |
| 2.8 | 1358 |

### Example 20: Preparation of Suspensions Containing Additives

Separation is often an issue when making biopolymer suspensions because some water can separate at the top of the suspension due to agglomeration and cohesion of the particles. Emulsifiers typically help to keep the oil and water phases together.

Different additives were tested for their ability to solve any separation issue that could occur in the biopolymer suspensions of the present invention.

Samples were assessed for separation, formation of agglomerates, and viscosity. The samples which showed no separation after suspension were tested with a centrifuge test for 10 minutes at 4000 RPM. The following additives were added to the cellulose suspension: Cetyl alcohol, Glyceryl stearate, PC90, PSC3, PEG, Guar, Xantham gum, Agarose, Sodium Hyaluronate, Tween 80™, Glycerol (humectant).

1) Cellulose

1.1) Tween 80™

The cellulose suspension was generated by milling cellulose in water with a cellulose to additive to water ratio of 1.5:1.25:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: <1 mL; formation of agglomerates: none; color change: none, still white; Viscosity: see Table 7.

TABLE 7
Viscosity of a cellulose suspension comprising
a Tween 80 ™ additive

| Shear Rate (Hz) | Viscosity (mPa · s) |
|---|---|
| 0.14 | 30330 |
| 0.28 | 20580 |
| 0.56 | 13580 |
| 1.4 | 8483 |
| 2.24 | 6917 |
| 2.8 | 6292 |

1.2) Glyceryl Stearate

The cellulose suspension was generated by milling cellulose in water with a cellulose to additive to water ratio of 1.5:1.25:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none, still white; Viscosity: see Table 8.

TABLE 8
Viscosity of a cellulose suspension comprising
a Glyceryl stearate additive

| Shear Rate (Hz) | Viscosity (mPa · s) |
|---|---|
| 0.14 | 253800 |
| 0.28 | 109900 |
| 0.56 | 61500 |
| 1.4 | 34600 |
| 2.24 | 27350 |
| 2.8 | 24790 |

1.3) Cetyl Alcohol

The cellulose suspension was generated by milling cellulose in water with a cellulose to additive to water ratio of 1.5:1.25:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: <1 mL; formation of agglomerates: none; color change: none, still white; Viscosity: see Table 9.

TABLE 9
Viscosity of a cellulose suspension
comprising a Cetyl alcohol additive

| Shear Rate (Hz) | Viscosity (mPa · s) |
|---|---|
| 0.14 | 114500 |
| 0.28 | 46330 |
| 0.56 | 22920 |
| 1.4 | 10630 |
| 2.24 | 7490 |
| 2.8 | 6192 |

1.4) Sodium Hyaluronate

The cellulose suspension was generated by milling cellulose in water with a cellulose to additive to water ratio of 1.5:1.25:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: light grey, still white; Viscosity: Too viscose to measure with the Brookfield viscometer with the spindle and chamber we have (max 1M mPa·s).

Another cellulose suspension was generated by milling cellulose in water with a cellulose to additive to water ratio of 1.5:0.2:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none, still white; Viscosity: see Table 10.

TABLE 10
Viscosity of a cellulose suspension comprising
a Sodium Hyaluronate additive

| Shear Rate (Hz) | Viscosity (mPa · s) |
|---|---|
| 0.14 | 106800 |
| 0.28 | 88420 |
| 0.56 | 60960 |
| 1.4 | 34870 |
| 2.24 | 26040 |
| 2.8 | 22920 |

1.5) PSC3

The cellulose suspension was generated by milling cellulose in water with a cellulose to additive to water ratio of 1.5:1.25:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none, still white; Viscosity: see Table 11.

TABLE 11
Viscosity of a cellulose suspension comprising a PSC3 additive

| Shear Rate (Hz) | Viscosity (mPa · s) |
|---|---|
| 0.14 | 190200 |
| 0.28 | 81670 |
| 0.56 | 47040 |
| 1.4 | 26570 |
| 2.24 | 21490 |
| 2.8 | 20560 |

1.6) PC90

The cellulose suspension was generated by milling cellulose in water with a cellulose to additive to water ratio of 1.5:1.30:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: ˜2 mL; formation of agglomerates: none; color change: none, still white; Viscosity: see Table 12.

TABLE 12
Viscosity of a cellulose suspension comprising a PC90 additive

| Shear Rate (Hz) | Viscosity (mPa · s) |
|---|---|
| 0.14 | 22000 |
| 0.28 | 9667 |
| 0.56 | 5708 |
| 1.4 | 7100 |
| 2.24 | 5563 |
| 2.8 | 3933 |

1.7) Agarose

The cellulose suspension was generated by milling cellulose in water with a cellulose to additive to water ratio of 1.5:0.5:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: light gray; Viscosity: formed solid gel, too viscose to measure with the Brookfield viscometer with the spindle and chamber we have (max 1M mPa·s).

Another cellulose suspension was generated by milling cellulose in water with a cellulose to additive to water ratio of 1.5:0.2:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none, still white; Viscosity: Too viscose to measure with the Brookfield viscometer with the spindle and chamber we have (max 1M mPa·s).

1.8) Xantham Gum

The cellulose suspension was generated by milling cellulose in water with a cellulose to additive to water ratio of 1.5:0.5:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none, still white; Viscosity: see Table 13.

TABLE 13
Viscosity of a cellulose suspension
comprising a Xantham gum additive

| Shear Rate (Hz) | Viscosity (mPa · s) |
|---|---|
| 0.14 | 483800 |
| 0.28 | 289000 |
| 0.56 | 180100 |

Another cellulose suspension was generated by milling cellulose in water with a cellulose to additive to water ratio of 1.5:0.2:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none, still white; Viscosity: see Table 14.

TABLE 14
Viscosity of a cellulose suspension
comprising a Xantham gum additive

| Shear Rate (Hz) | Viscosity (mPa · s) |
|---|---|
| 0.14 | 233300 |
| 0.28 | 121400 |
| 0.56 | 70250 |
| 1.4 | 39450 |
| 2.24 | 31530 |
| 2.8 | 29440 |

1.9) PEG 20K

The cellulose suspension was generated by milling cellulose in water with a cellulose to additive to water ratio of 1.5:0.5:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: <1 mL; formation of agglomerates: none; color change: none, still white; Viscosity: see Table 15.

TABLE 15
Viscosity of a cellulose suspension comprising a PEG 20K additive

| Shear Rate (Hz) | Viscosity (mPa · s) |
|---|---|
| 0.14 | 104300 |
| 0.28 | 61170 |
| 0.56 | 36460 |
| 1.4 | 21570 |
| 2.24 | 17450 |
| 2.8 | 16720 |

1.10) Glycerol

The cellulose suspension was generated by milling cellulose in water with a cellulose to additive to water ratio of 1.5:1.25:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: <1 mL; formation of agglomerates: none; color change: none, still white; Viscosity: see Table 16.

TABLE 16
Viscosity of a cellulose suspension comprising a Glycerol additive

| Shear Rate (Hz) | Viscosity (mPa · s) |
|---|---|
| 0.14 | 104300 |
| 0.28 | 60080 |
| 0.56 | 34130 |
| 1.4 | 17900 |
| 2.24 | 14010 |
| 2.8 | 12620 |

1.11) Guar

The cellulose suspension was generated by milling cellulose in water with a cellulose to additive to water ratio of 1.5:0.15:20 at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: grey; Viscosity: see Table 17.

TABLE 17
Viscosity of a cellulose suspension comprising a Guar additive

| Shear Rate (Hz) | Viscosity (mPa · s) |
|---|---|
| 0.14 | 173000 |
| 0.28 | 87500 |
| 0.56 | 46750 |
| 1.4 | 20970 |
| 2.24 | 14310 |
| 2.8 | 12510 |

2) Chitin

2.1) Cetyl Alcohol

The chitin suspension was generated by milling chitin in water with a chitin to water ratio of 1:20 at 670 RPM with fifty units of 10 mm ball using the 10+1 Alt method, where it is milled for ten minutes followed by a pause for one minute then milling for ten minutes in the opposite direction for a total of 3 hours. Cetyl alcohol was added to create a chitin to cetyl alcohol to water ratio of 1:1.25:20 then milled under the same conditions for 3 hours.

Results: phase separation: ˜2 mL; formation of agglomerates: none; color change: light grey; Viscosity: see Table 18.

TABLE 18
Viscosity of a chitin suspension comprising a cetyl alcohol additive

| Shear Rate (Hz) | Viscosity (mPa · s) |
|---|---|
| 0.14 | 315500 |
| 0.28 | 137800 |
| 0.56 | 71880 |
| 1.4 | 31680 |
| 2.24 | 19670 |
| 2.8 | 15300 |

2.2) Glyceryl Stearate

The chitin suspension was generated by milling chitin in water with a chitin to water ratio of 1:20 at 670 RPM with fifty units of 10 mm ball using the 10+1 Alt method, where it is milled for ten minutes followed by a pause for one minute then milling for ten minutes in the opposite direction for a total of 3 hours. Cetyl alcohol was added to create a chitin to glyceryl stearate water ratio of 1:1.25:20 then milled under the same conditions for 3 hours.

Results: phase separation: none; formation of agglomerates: none; color change: none, still white; Viscosity: see Table 19.

TABLE 19
Viscosity of a chitin suspension comprising
a glyceryl stearate additive

| Shear Rate (Hz) | Viscosity (mPa · s) |
|---|---|
| 0.14 | 567700 |
| 0.28 | 265600 |
| 0.56 | 148100 |
| 1.4 | 79380 |
| 2.24 | 60070 |
| 2.8 | 49250 |

3) Chitosan

3.1) Cetyl Alcohol

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The chitosan suspension was generated by milling chitosan in water with a chitosan to water ratio of 1.3:20 at 670 RPM with fifty units of 10 mm ball using the 10+1 Alt method, where it is milled for ten minutes followed by a pause for one minute then milling for ten minutes in the opposite direction for a total of 3 hours. Cetyl alcohol was added to create a chitosan to cetyl alcohol to water ratio of 1:1.25:20 then milled under the same conditions for 3 hours.

Results: phase separation: none; formation of agglomerates: none; color change: none, still off-white; Viscosity: see Table 20.

TABLE 20
Viscosity of a chitosan suspension
comprising a cetyl alcohol additive

| Shear Rate (Hz) | Viscosity (mPa · s) |
|---|---|
| 0.14 | 512000 |
| 0.28 | 331300 |
| 0.56 | 169500 |
| 1.4 | 60780 |
| 2.24 | 36490 |
| 2.8 | 29060 |

3.2) Glyceryl Stearate

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The chitosan suspension was generated by milling chitosan in water with a chitosan to water ratio of 1.3:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate was added to create a chitosan to glyceryl stearate to water ratio of 1:1.25:20 then milled under the same conditions for 3 hours.

Results: phase separation: none; formation of agglomerates: none; color change: none, still off-white; Viscosity: see Table 21.

TABLE 21
Viscosity of a chitosan suspension comprising
a Glyceryl stearate additive

| Shear Rate (Hz) | Viscosity (mPa · s) |
|---|---|
| 0.14 | 197000 |
| 0.28 | 92420 |
| 0.56 | 48460 |
| 1.4 | 19370 |
| 2.24 | 10830 |
| 2.8 | 8275 |

4) Centrifuge Separation Tests

Samples were centrifuged for 10 minutes at 4000 RPM. The results were as follows:
Cellulose with glyceryl stearate <200 μL separated;
Cellulose with cetyl alcohol ˜5 mL separated;
Cellulose with PSC3, no separation;
Cellulose with xantham gum, no separation;
Cellulose with Sodium Hyaluronate, no separation;
Cellulose with Sodium Hyaluronate, ˜6 mL separation of gel not just water;
Chitin with cetyl alcohol, ˜4 mL separated;
Chitin with glyceryl stearate, no separation;
Chitosan with cetyl alcohol, ˜2 mL separated;
Chitosan with glyceryl stearate, ˜2 mL separated

Conclusions

Several additives appear adequate to reduce or eliminate any phase separation that may be observed in the original water and biopolymers formulations. Glyceryl stearate, cetyl alcohol, tara gum, sodium hyaluronate, PSC3, xantham gum, and guar appear to stabilize the suspension with adequate amounts of the additives used. Overall viscosities of the formulations containing additives were noted to be significantly increased, which could be mitigated by the addition of other additives. With further separation testing via centrifugation (4000 RPM for 10 minutes), glyceryl stearate, PSC3, xantham gum and sodium hyaluronate blends were shown to persist in their stable state.

### Example 21: Flower Based Suspensions

Lavender, chrysanthemum, rosebud, jasmine and calendula flowers were transformed into suspensions containing only the flower and water.

For all samples herein, the flowers were acquired dry. The dry flowers were ground to smaller particles in a blade grinder for 30 seconds.

The dry, ground flower particles were pre-milled dry at 670 RPM with 50 units of 10 mm ball using the 10+1 Alt method, where it is milled for ten minutes followed by a pause for one minute then milling for ten minutes in the opposite direction for a total of 1 hour, to produce a fine powder.

The flower suspension was generated by milling flower powder in water in a ratio of 2.00:20 at 670 RPM with fifty units of 10 mm ball using the 10+1 Alt method, where it is milled for ten minutes followed by a pause for one minute then milling for ten minutes in the opposite direction for a total of 1 hour.

1) Lavender: Appearance of the suspension: homogenous dark green. Viscosity: see Table 22.

TABLE 22
Viscosity of a Lavender suspension 2:20

| Shear Rate (Hz) | Viscosity (mPa · s) |
|---|---|
| 0.14 | 278200 |
| 0.28 | 149200 |
| 0.56 | 81670 |
| 1.4 | 33050 |
| 2.24 | 16340 |
| 2.8 | 12670 |

2) Chrysanthemum: Appearance of the suspension: homogenous dark beige. Viscosity: see Table 23.

TABLE 23
Viscosity of a Chrysanthemum suspension 2:20

| Shear Rate (Hz) | Viscosity (mPa · s) |
|---|---|
| 0.14 | 287000 |
| 0.28 | 141000 |
| 0.56 | 79290 |
| 1.4 | 40430 |
| 2.24 | 28740 |
| 2.8 | 22910 |

3) Rosebud: Appearance of the suspension: homogenous yellow/beige. Viscosity: see Table 24.

TABLE 24
Viscosity of a Rosebud suspension 2:20

| Shear Rate (Hz) | Viscosity (mPa · s) |
|---|---|
| 0.14 | 79500 |
| 0.28 | 48830 |
| 0.56 | 27630 |
| 1.4 | 13130 |
| 2.24 | 9052 |
| 2.8 | 7567 |

4) Jasmine: Appearance of the suspension: homogenous brown. Viscosity: see Table 25.

TABLE 25
Viscosity of a Jasmine suspension 2:20

| Shear Rate (Hz) | Viscosity (mPa · s) |
|---|---|
| 0.14 | 5500 |
| 0.28 | 4583 |
| 0.56 | 3792 |
| 1.4 | 2367 |
| 2.24 | 1656 |
| 2.8 | 1417 |

5) Calendula: Appearance of the suspension: homogenous dark mustard. Viscosity: see Table 26.

TABLE 26
Viscosity of a Calendula suspension 2:20

| Shear Rate (Hz) | Viscosity (mPa · s) |
|---|---|
| 0.14 | 279700 |
| 0.28 | 145900 |
| 0.56 | 81080 |
| 1.4 | 40170 |
| 2.24 | 28640 |
| 2.8 | 24630 |

Conclusions: Advantageously, dry flowers can be a suitable material for producing homogenous suspensions with adequate viscosities. The smells overall are still pleasant, immediately after production. A preservative may be preferable to stabilize the suspensions for long-term storage.

### Example 22: Freeze/Thaw Pretreatment

Freeze/thawing was tested as a pre-treatment technique prior to milling because it has the potential to disrupt hydrogen bonding between the polymer chains, thereby, increasing the swell of the biopolymer.

The biopolymer was wetted then frozen at −15° C. for 10 hours prior to being thawed. This freeze/thaw cycle was repeated 2 times. The processed biopolymer was then milled to suspend.

The biopolymer was combined with at least enough water until wet and saturated with water. The mixture was frozen at −15° C. for 10 hours then thaw. This freeze/thaw cycle was repeated 2 times. The processed biopolymer was then milled to suspend. Visual observational results were noted for the following: phase separation, formation of agglomerates, color change and viscosity.

1) Chitin

Freezing pre-treatment of a Chitin mixture was prepared with additional water to create a 1:20 ratio suspension. The mixture was milled at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: from beige to off-white; Viscosity: see Table 27.

TABLE 27
Viscosity of a chitin suspension following
a frozen/thaw pre-treatment

| Shear Rate (Hz) | Viscosity (mPa · s) |
|---|---|
| 0.14 | 139000 |
| 0.28 | 62830 |
| 0.56 | 30710 |
| 1.4 | 13200 |
| 2.24 | 8740 |
| 2.8 | 7158 |

2) Chitosan

Freezing pre-treatment of a Chitosan mixture was prepared with additional water to create a 1.30:20 ratio suspension. The mixture was milled at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: from beige to beige-white; Viscosity: see Table 28.

TABLE 28
Viscosity of a Chitosan suspension following
a frozen/thaw pre-treatment

| Shear Rate (Hz) | Viscosity (mPa · s) |
|---|---|
| 0.14 | 417000 |
| 0.28 | 193400 |
| 0.56 | 82580 |
| 1.4 | 35480 |
| 2.24 | 21830 |
| 2.8 | 17150 |

3) Cellulose

Freezing pre-treatment a cellulose mixture was prepared with additional water to create a 1:20 ratio suspension. The mixture was milled at 670 RPM with fifty units of 10 mm ball using the 10+1 Alt method, where it is milled for ten minutes followed by a pause for one minute then milling for ten minutes in the opposite direction for a total of 3 hours.

Results: phase separation: none; formation of agglomerates: none; color change: none; Viscosity: see Table 29.

TABLE 29
Viscosity of a Cellulose suspension
following a frozen/thaw pre-treatment

| Shear Rate (Hz) | Viscosity (mPa · s) |
|---|---|
| 0.14 | 53000 |
| 0.28 | 22170 |
| 0.56 | 13630 |
| 1.4 | 12880 |
| 2.24 | 8573 |
| 2.8 | 7058 |

Conclusions

The freezing pre-treatment had a decrease of ˜18% on the viscosity of chitin, an increase of 115% on the viscosity of chitosan and an increase of ˜25% on the viscosity of cellulose. The increase in viscosity could be a result of polymer separation and the decrease in viscosity could be a result of polymer chain breakage.

### Example 23: Low Energy Milling for Chitin, Chitosan and Cellulose Suspensions

The particle size at the crossover point of biopolymer suspension was investigated using low energy milling. This allowed for particle size analysis of poorly suspended samples and identification of morphology change of the particles or fibers.

The analysis was done by milling at low RPM. Aliquots were removed during the milling process at different time intervals. Horiba particle size analysis and SEM imaging were used to analyze particle size and morphology, respectively.

The biopolymer was suspended with the planetary mill in a 1:20 ratio for chitin, 1.30:20 ratio for chitosan and 1.5:20 ratio for cellulose at 200 RPM and 400 RPM, with 10 units of 10 mm.

1) Chitin

Chitin was milled for different durations with different power outputs to see the effect on suspension and particle size. Chitin particle size was also measured in water without milling and after generation of the fully suspended version. Table 30 summarizes the results, with milling conditions described in sections below

TABLE 30
Particle size of chitin under different milling conditions

| | Milling conditions* | | | |
|---|---|---|---|---|
| Chitin | C0 | C18 | C6 | CF |
| Particle Size (μm) | 218.7 | 181.8 | 172.2 | 110.3 |
| min (μm) | 15.56 | 11 | 9.25 | 1.156 |
| max (μm) | 418.6 | 418.6 | 418.6 | 74 (large spike at~160 was |
| | | | | ignored) |

*C0 = no mill; C18 = 200 RPM; C6 = 400 RPM; CF = Standard mill

1.1) Chitin Milling at 200 RPM (C18)

The chitin suspension was generated by milling chitin in water with a chitin to water ratio of 1:20 ratio at 200 RPM with 10 units of 10 mm ball in ten-minute increments, where aliquots were removed for imaging at 10, 20, 30, 60 and 180 minutes.

Suspension appearance: fluffed polymer but separated, not fully suspended. Particle Size analysis: Number average particle size: 181.8 μm; Particle size range: 11.00-418.6 μm. Details of the measurements are depicted in FIG. 38A and Table 31. SEM imaging is shown in FIG. 38B, the picture showing some larger particles with smaller agglomerated particles.

TABLE 31
Particle Size analysis for Chitin milling at 200 RPM (C18)

| | |
|---|---|
| Summary | Percentiles |

| Data | Value | % Tile | Size(um) |
|---|---|---|---|
| MV(um): | 181.8 | 10.00 | 37.31 |
| MN(um): | 20.08 | 20.00 | 59.15 |
| MA(um): | 85.29 | 30.00 | 83.97 |
| CS: | 7e−2 | 40.00 | 115.8 |
| SD: | 143.2 | 50.00 | 158.5 |
| Mz: | 181.9 | 60.00 | 221.0 |
| si: | 126.8 | 70.00 | 253.5 |
| Ski: | 0.2569 | 80.00 | 306.5 |
| Kg: | 0.727 | 90.00 | 367.8 |
| | | 95.00 | 389.5 |

1.2) Chitin Milling at 400 RPM (C6)

The chitin suspension was generated by milling chitin in water with a chitin to water ratio of 1:20 ratio at 400 RPM with 10 units of 10 mm ball in ten-minute increments, where aliquots were removed for imaging at 10, 30 and 60 minutes.

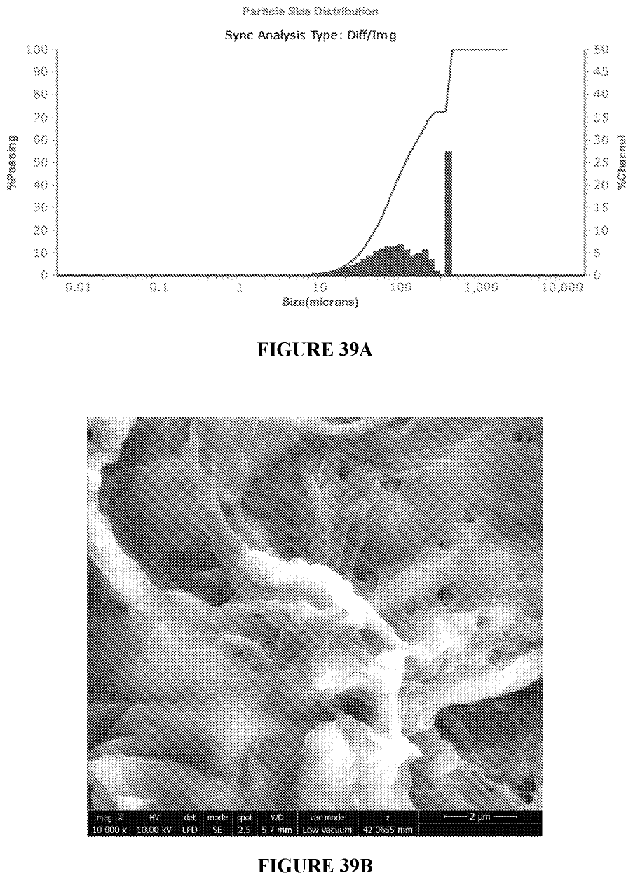

Suspension appearance: Partially suspended, ˜15% separation. Particle Size analysis: Number average particle size: 172.2 μm; Particle size range: 9.25-418.6 μm. Details of the measurements are depicted in FIG. 39A and Table 32. SEM imaging is shown in FIG. 39B.

TABLE 32
Particle Size analysis for Chitin milling at 400 RPM (C6)

| | |
|---|---|
| Summary | Percentiles |

| Data | Value | % Tile | Size(um) |
|---|---|---|---|
| MV(um): | 172.2 | 10.00 | 30.76 |
| MN(um): | 17.78 | 20.00 | 47.16 |
| MA(um): | 69.81 | 30.00 | 63.76 |
| CS: | 8.6e−2 | 40.00 | 84.12 |
| SD: | 169.2 | 50.00 | 110.2 |
| Mz: | 176.7 | 60.00 | 159.1 |
| si: | 142.5 | 70.00 | 223.3 |
| Ski: | 0.563 | 80.00 | 370.8 |
| Kg: | 0.516 | 90.00 | 392.0 |
| | | 95.00 | 403.6 |

1.3) Chitin No Mill (C0)

For reference, particle size analysis of water and chitin was conducted. Chitin and water were combined in a 1:20 ratio without any milling.

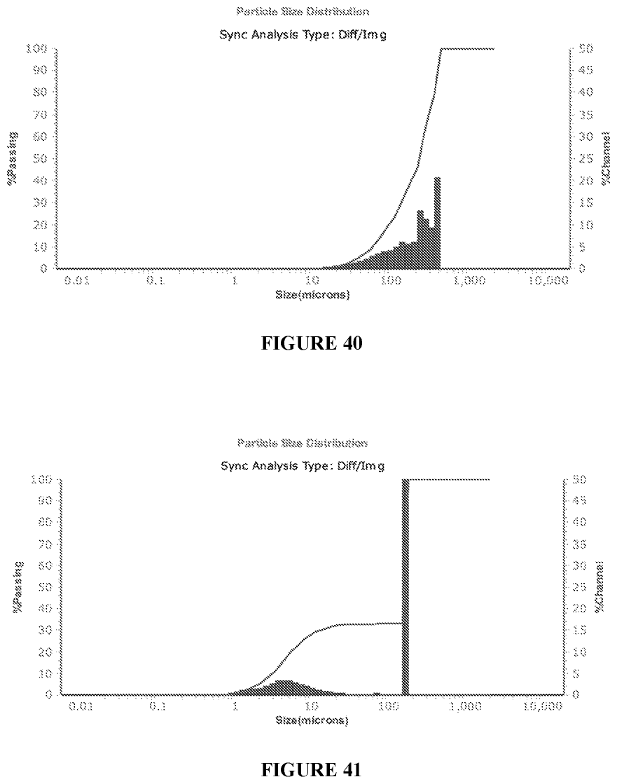

Particle Size analysis: Number average particle size: 218.7 μm; Particle size range: 15.56-418.6 μm. Details of the measurements are depicted in FIG. 40 and Table 33.

TABLE 33
Particle Size analysis for Chitin no mill (C0)

| | |
|---|---|
| Summary | Percentiles |

| Data | Value | % Tile | Size(um) |
|---|---|---|---|
| MV(um): | 218.7 | 10.00 | 56.86 |
| MN(um): | 28.49 | 20.00 | 92.50 |
| MA(um): | 122.9 | 30.00 | 132.5 |
| CS: | 4.9e−2 | 40.00 | 178.0 |
| SD: | 144.2 | 50.00 | 222.6 |
| Mz: | 222.2 | 60.00 | 252.6 |
| si: | 126.6 | 70.00 | 295.5 |
| Ski: | −0.01444 | 80.00 | 354.1 |
| Kg: | 0.681 | 90.00 | 382.8 |
| | | 95.00 | 398.0 |

1.4) Chitin Standard Mill (CF)

The chitin suspension was generated by milling chitin in water with a chitin to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours.

Suspension appearance: Fully suspended. Particle Size analysis: Number average particle size: 110.3 μm; Particle size range: 1.156-74 μm. Details of the measurements are depicted in FIG. 41 and Table 34.

TABLE 34
Particle Size analysis for Chitin standard mill (CF)

| | |
|---|---|
| Summary | Percentiles |

| Data | Value | % Tile | Size(um) |
|---|---|---|---|
| MV(um): | 110.3 | 10.00 | 3.06 |
| MN(um): | 1.583 | 20.00 | 5.31 |
| MA(um): | 9.93 | 30.00 | 12.45 |
| CS: | 6.04e−1 | 40.00 | 151.2 |
| SD: | 81.81 | 50.00 | 155.5 |
| Mz: | 109.2 | 60.00 | 159.1 |
| si: | 66.87 | 70.00 | 162.7 |
| Ski: | −0.82018 | 80.00 | 156.4 |
| Kg: | 0.447 | 90.00 | 170.8 |
| | | 95.00 | 173.4 |

2) Chitosan

Chitosan was milled at different times with different power outputs to see the effect on suspension and particle size. Chitosan particle size was also measured in water without milling and as the fully suspended version. Table 35 summarizes the results, with milling conditions described below.

TABLE 35
Particle size of chitosan under different milling conditions

Milling conditions*

| Chitosan | B0 | B18 | B6 | BF |
|---|---|---|---|---|
| Particle Size (μm) | 65.76 | 92.78 | 95.83 | 32.99 |
| min (μm) | 7.78 | 5.5 | 7.78 | 3.89 |
| max (μm) | 248.9 | 248.9 | 296 | 148 |

*B0 = no mill; B18 = 200 RPM; B6 = 400 RPM; BF = Standard mill

1.1) Chitosan Milling at 200 RPM (B18)

The chitosan suspension was generated by milling chitosan in water with a chitosan to water ratio of 1.3:20 ratio at 200 RPM with 10 units of 10 mm ball in ten-minute increments, where aliquots were removed for imaging at 10, 20, 30, 60 and 180 minutes.

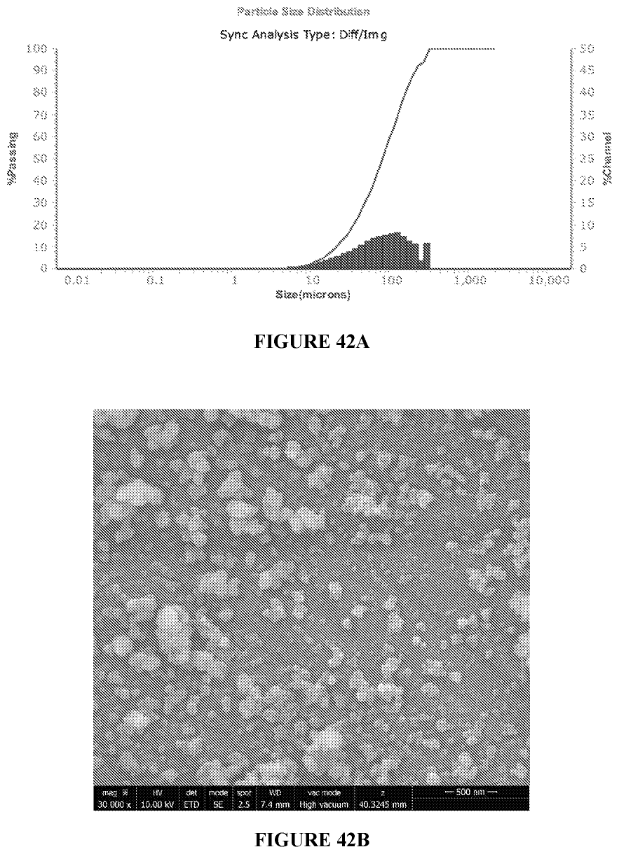

Suspension appearance: fluffed polymer but separated, not fully suspended. Particle Size analysis: Number average particle size: 92.78 μm; Particle size range: 5.50-248.9 μm. Details of the measurements are depicted in FIG. 42A and Table 36. SEM imaging is shown in FIG. 42B, the picture showing small nano sized particles.

TABLE 36
Particle Size analysis for chitosan milling at 200 RPM (B18)

| | |
|---|---|
| Summary | Percentiles |

| Data | Value | % Tile | Size(um) |
|---|---|---|---|
| MV(um): | 92.78 | 10.00 | 19.60 |
| MN(um): | 10.23 | 20.00 | 32.12 |
| MA(um): | 43.84 | 30.00 | 45.04 |
| CS: | 1.37e−1 | 40.00 | 58.76 |
| SD: | 67.68 | 50.00 | 74.72 |
| Mz: | 88.07 | 60.00 | 93.61 |
| si: | 70.58 | 70.00 | 115.8 |
| Ski: | 0.394 | 80.00 | 145.8 |
| Kg: | 1.095 | 90.00 | 191.8 |
| | | 95.00 | 255.7 |

2.2) Chitosan Milling at 400 RPM (B6)

The chitosan suspension was generated by milling chitosan in water with a chitosan to water ratio of 1:20 ratio at 400 RPM with 10 units of 10 mm ball in ten-minute increments, where aliquots were removed for imaging at 10, 30 and 60 minutes.

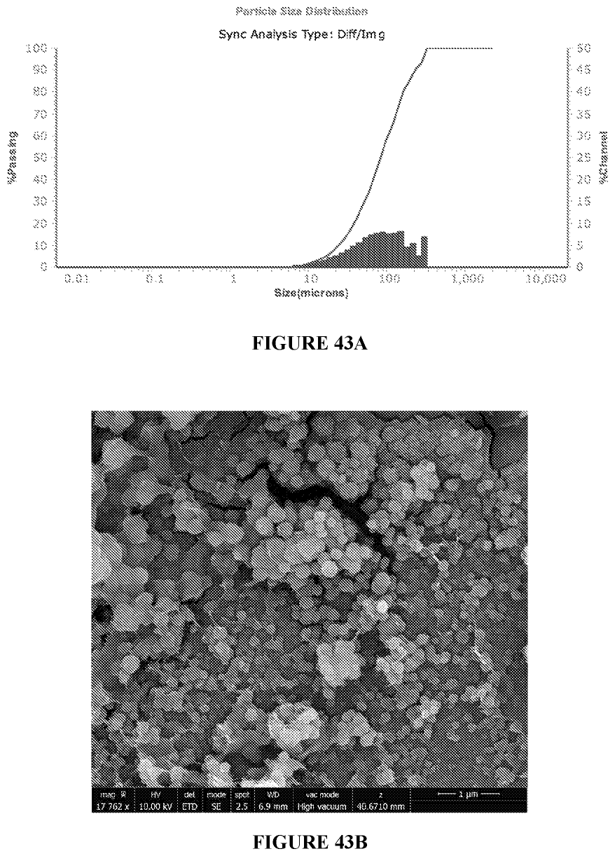

Suspension appearance: Partially suspended, ˜15% separation. Particle Size analysis: Number average particle size: 95.83 μm; Particle size range: 7.78-296 μm. Details of the measurements are depicted in FIG. 43A and Table 37. SEM imaging is shown in FIG. 43B, the picture showing nano sized particles.

TABLE 37
Particle Size analysis for Chitosan milling at 400 RPM (B6)

| | |
|---|---|
| Summary | Percentiles |

| Data | Value | % Tile | Size(um) |
|---|---|---|---|
| MV(um): | 95.83 | 10.00 | 22.08 |
| MN(um): | 12.28 | 20.00 | 34.71 |
| MA(um): | 47.68 | 30.00 | 47.03 |
| CS: | 1.26e−1 | 40.00 | 60.17 |
| SD: | 69.57 | 50.00 | 75.17 |
| Mz: | 91.26 | 60.00 | 93.59 |
| si: | 72.13 | 70.00 | 118.0 |
| Ski: | 0.430 | 80.00 | 146.0 |
| Kg: | 1.120 | 90.00 | 206.7 |
| | | 95.00 | 261.6 |

2.3) Chitosan No Mill (B0)

For reference, particle size analysis of water and chitin was conducted. Chitin and water were combined in a 1:20 ratio without any milling.

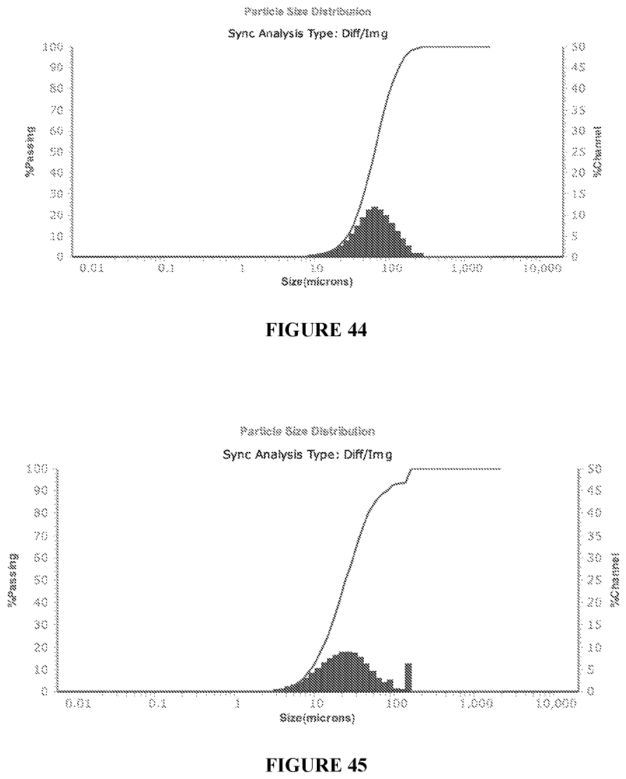

Number average particle size: 65.76 μm. Particle size range: 7.78-248.9 μm. Details of the measurements are depicted in FIG. 44 and Table 38.

TABLE 38
Particle Size analysis for Chitosan no mill (B0)

| | |
|---|---|
| Summary | Percentiles |

| Data | Value | % Tile | Size(um) |
|---|---|---|---|
| MV(um): | 65.76 | 10.00 | 24.78 |
| MN(um): | 17.37 | 20.00 | 33.78 |
| MA(um): | 44.91 | 30.00 | 41.52 |
| CS: | 1.34e−1 | 40.00 | 48.94 |
| SD: | 35.60 | 50.00 | 56.80 |
| Mz: | 62.94 | 60.00 | 65.90 |
| si: | 36.67 | 70.00 | 77.22 |
| Ski: | 0.324 | 80.00 | 92.88 |
| Kg: | 1.098 | 90.00 | 119.4 |
| | | 95.00 | 143.4 |

2.4) Chitosan Standard Mill (BF)

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The CXC chitosan suspension was generated by milling chitosan in water with a chitosan to water ratio of 1.30:20 at 670 RPM with fifty units of 10 mm ball using the 10+1 Alt method, where it is milled for ten minutes followed by a pause for one minute then milling for ten minutes in the opposite direction for a total of 3 hours.

Suspension appearance: Fully suspended. Particle Size analysis: Number average particle size: 32.99 μm; Particle size range: 3.89-148.0 μm. Details of the measurements are depicted in FIG. 45 and Table 39.

TABLE 39
Particle Size analysis for Chitosan standard mill (BF)

| | |
|---|---|
| Summary | Percentiles |

| Data | Value | % Tile | Size(um) |
|---|---|---|---|
| MV(um): | 32.99 | 10.00 | 8.15 |
| MN(um): | 6.45 | 20.00 | 11.58 |
| MA(um): | 16.78 | 30.00 | 14.87 |
| CS: | 3.58e−1 | 40.00 | 18.40 |
| SD: | 19.74 | 50.00 | 22.43 |
| Mz: | 27.47 | 60.00 | 27.29 |
| si: | 28.61 | 70.00 | 33.50 |
| Ski: | 0.560 | 80.00 | 43.20 |
| Kg: | 2.075 | 90.00 | 72.36 |
| | | 95.00 | 129.8 |

3) Cellulose

Cellulose was milled at different times with different power outputs to see the effect on suspension and particle size. Cellulose particle size was also measured in water without milling and as the fully suspended version. Table 40 summarizes the results, with milling conditions described below.

TABLE 40
Particle size of cellulose under different milling conditions

Milling conditions*

| Cellulose | A0 | A18 | A6 | AF |
|---|---|---|---|---|
| Particle Size (μm) | 82.83 | 81.19 | 82.72 | 1.117 |
| min (μm) | 11 | 9.25 | 7.72 | 0.578 |
| max (μm) | 296 | 296 | 296 | 124.5 |

*A0 = no mill; A18 = 200 RPM; A6 = 400 RPM; AF = Standard mill

3.1) Cellulose Milling at 200 RPM (A18)

The cellulose suspension was generated by milling cellulose in water with a cellulose to water ratio of 1:20 ratio at 200 RPM with 10 units of 10 mm ball in ten-minute increments, where aliquots were removed for imaging at 10, 20, 30, 60 and 180 minutes.

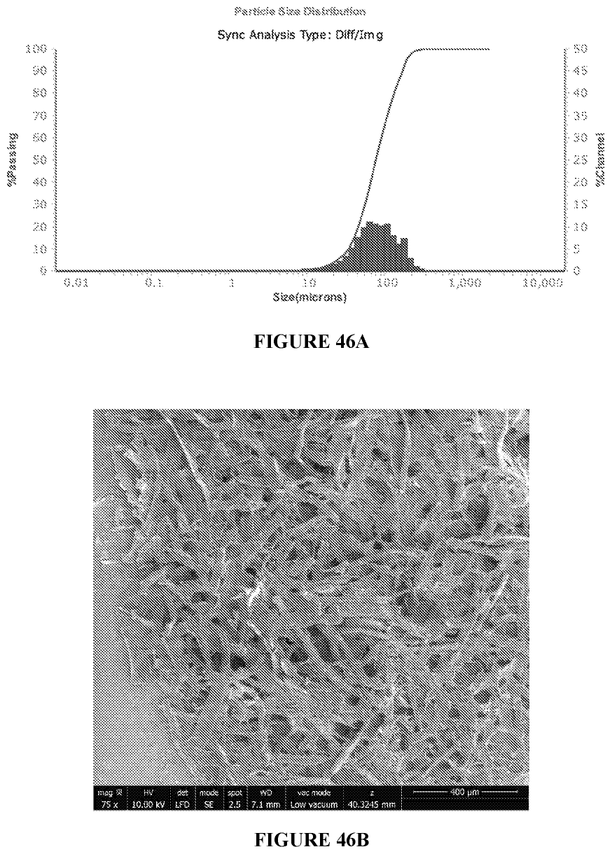

Suspension appearance: fluffed polymer but separated, not fully suspended. Particle Size analysis: Number average particle size: 81.19 μm; Particle size range: 9.25-296 μm. Details of the measurements are depicted in FIG. 46A and Table 41. SEM imaging is shown in FIG. 46B, the picture showing large fibers 20-62 μm wide.

TABLE 41
Particle Size analysis for cellulose milling at 200 RPM (B18)

| | |
|---|---|
| Summary | Percentiles |

| Data | Value | % Tile | Size(um) |
|---|---|---|---|
| MV(um): | 81.19 | 10.00 | 31.15 |
| MN(um): | 19.87 | 20.00 | 41.78 |
| MA(um): | 54.97 | 30.00 | 50.39 |
| CS: | 1.09e−1 | 40.00 | 59.15 |
| SD: | 46.18 | 50.00 | 69.48 |
| Mz: | 79.26 | 60.00 | 82.45 |
| si: | 45.73 | 70.00 | 97.24 |
| Ski: | 0.347 | 80.00 | 117.5 |
| Kg: | 1.023 | 90.00 | 153.7 |
| | | 95.00 | 172.4 |

3.2) Cellulose Milling at 400 RPM (A6)

The cellulose suspension was generated by milling cellulose in water with a cellulose to water ratio of 1:20 ratio at 400 RPM with 10 units of 10 mm ball in ten-minute increments, where aliquots were removed for imaging at 10, 30 and 60 minutes.

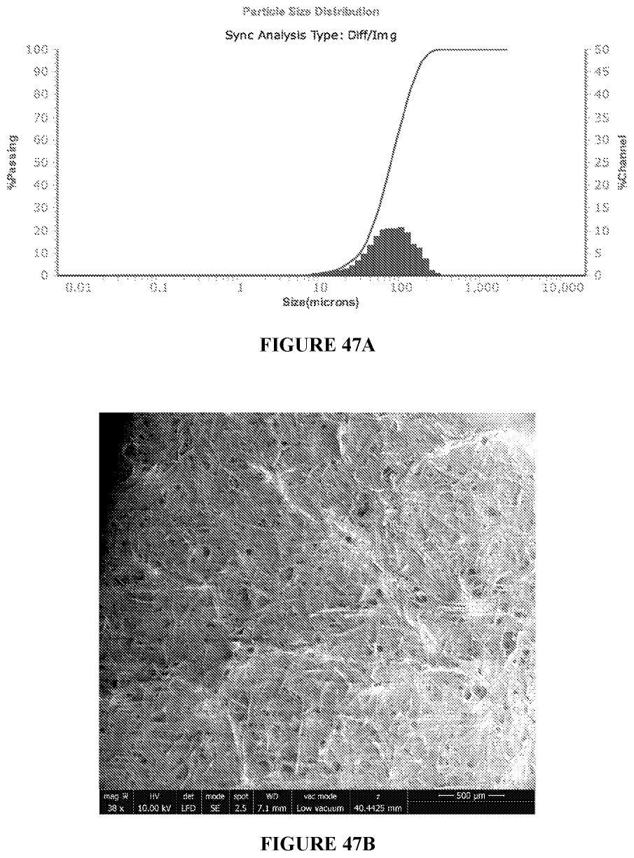

Suspension appearance: Partially suspended, ˜15% separation. Particle Size analysis: Number average particle size: 82.72 μm. Particle size range: 7.72-296 μm. Details of the measurements are depicted in FIG. 47A and Table 42. SEM imaging is shown in FIG. 47B, the picture showing large fibers 4-16 μm wide.

TABLE 42
Particle Size analysis for Cellulose milling at 400 RPM (A6)

| | |
|---|---|
| Summary | Percentiles |

| Data | Value | % Tile | Size(um) |
|---|---|---|---|
| MV(um): | 82.72 | 10.00 | 28.72 |
| MN(um): | 16.26 | 20.00 | 41.71 |
| MA(um): | 53.83 | 30.00 | 51.45 |
| CS: | 1.11e−1 | 40.00 | 61.17 |
| SD: | 46.89 | 50.00 | 72.26 |
| Mz: | 80.27 | 60.00 | 85.46 |
| si: | 47.14 | 70.00 | 100.7 |
| Ski: | 0.300 | 80.00 | 120.3 |
| Kg: | 1.018 | 90.00 | 153.2 |
| | | 95.00 | 177.4 |

3.3) Cellulose No Mill (A0)

For reference, particle size analysis of water and chitin was conducted. Cellulose and water were combined in a 1:20 ratio without any milling.

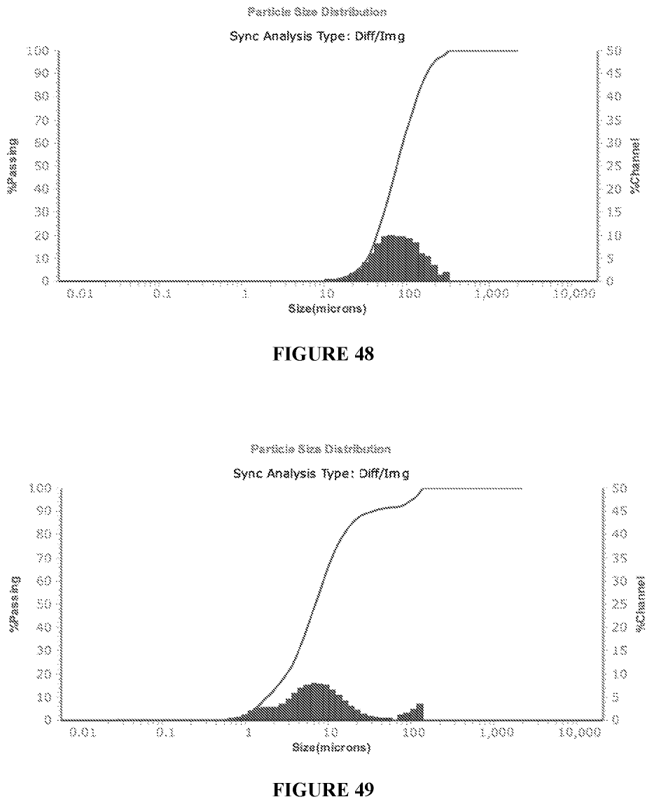

Suspension appearance: Fully separated. Particle Size analysis: Number average particle size: 82.83 μm; Particle size range: 11-296 μm. Details of the measurements are depicted in FIG. 48 and Table 43.

TABLE 43
Particle Size analysis for Cellulose no mill (A0)

| | |
|---|---|
| Summary | Percentiles |

| Data | Value | % Tile | Size(um) |
|---|---|---|---|
| MV(um): | 82.83 | 10.00 | 30.08 |
| MN(um): | 23.81 | 20.00 | 39.56 |
| MA(um): | 55.02 | 30.00 | 48.02 |
| CS: | 1.09e−1 | 40.00 | 57.17 |
| SD: | 47.73 | 50.00 | 68.17 |
| Mz: | 78.57 | 60.00 | 81.55 |
| si: | 49.09 | 70.00 | 97.95 |
| Ski: | 0.395 | 80.00 | 119.2 |
| Ka: | 1.066 | 90.00 | 157.8 |
| | | 95.00 | 190.0 |

3.4) Cellulose Standard Mill (AF)

The CXC cellulose suspension was generated by milling cellulose in water with a cellulose to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours.

Suspension appearance: Fully suspended. Particle Size analysis: Number average particle size: 1.117 μm; Particle size range: 0.578-124.5 μm. Details of the measurements are depicted in FIG. 49 and Table 44.

TABLE 44
Particle Size analysis for Cellulose standard mill (AF)

| | |
|---|---|
| Summary | Percentiles |

| Data | Value | % Tile | Size(um) |
|---|---|---|---|
| MV(um): | 15.01 | 10.00 | 1.663 |
| MN(um): | 1.117 | 20.00 | 2.884 |
| MA(um): | 4.04 | 30.00 | 3.98 |
| CS: | 1.486 | 40.00 | 5.08 |
| SD: | 7.06 | 50.00 | 6.32 |
| Mz: | 8.41 | 60.00 | 7.88 |
| si: | 17.48 | 70.00 | 10.01 |
| Ski: | 0.666 | 80.00 | 13.71 |
| Kg: | 4.67 | 90.00 | 29.60 |
| | | 95.00 | 93.32 |

Conclusion

Overall, there appears to be a maximum particle size for the biopolymers, where, when reduced produces a suspension.

### Example 24: Oil Incorporation into Chitin Chitosan and Cellulose Suspension

In cosmetics, the inclusion of oils is common. The stability of the mixture can be affected by the amount of oil added to a system. A base material that can accommodate a high quantity of oil improves applicability and would reduce the amount of emulsifier needed to maintain the integrity of the suspension.

For the biopolymer suspensions, the oil quantity was modified from 10% and higher of overall liquid content to test the effect of overall oil concentration on the stability of the suspensions. The suspensions were produced with the planetary mill.

The biopolymer was suspended with the planetary mill in a 1:20 ratio for chitin, 1.30:20 ratio for chitosan and 1.5:20 ratio for cellulose, where the liquid content was varied from 90% water/10% oil, up to 50% water/50% oil.

The viscosity of the samples was measured with a Brookfield RVDVNX Rheometer. Visual observational results were noted for the following: phase separation, formation of agglomerates, color change.

1) Chitin with Oil

The chitin suspension was generated by milling chitin in water with oil in a ratio of either, 1:18:2 (10% oil), 1:16:4 (20% oil), 1:14:6 (30% oil), 1:12:8 (40% oil) or 1:10:10 (50% oil) at 670 RPM with fifty units of 10 mm ball using the 10+1 Alt method, where it is milled for ten minutes followed by a pause for one minute then milling for ten minutes in the opposite direction for a total of 3 hours.

1.1) Chitin 10% Oil in Water

Results: Phase separation: No. Formation of agglomerates: homogeneous appearance. Color: off-white. Viscosity: see Table 45.

TABLE 45
Viscosity of a Chitin 10% oil in water suspension

| Shear Rate (Hz) | Viscosity (mPa · s) |
|---|---|
| 0.14 | 148200 |
| 0.28 | 75170 |
| 0.56 | 40210 |
| 1.4 | 17250 |
| 2.24 | 11890 |
| 2.8 | 9850 |

1.2) Chitin 20% Oil in Water

Results: phase separation: tiny amount ˜30 μL. Formation of agglomerates: homogeneous appearance. Color: off-white. Viscosity: see Table 46.

### Viscosity:

TABLE 46
Viscosity of a Chitin 20% oil in water suspension

| Shear Rate (Hz) | Viscosity (mPa · s) |
|---|---|
| 0.14 | 258500 |
| 0.28 | 101200 |
| 0.56 | 47040 |
| 1.4 | 19720 |
| 2.24 | 14150 |
| 2.8 | 12820 |

1.3) Chitin 30% Oil in Water

Results: phase separation: tiny amount ˜30 μL. Formation of agglomerates: homogeneous appearance. Color: off-white. Viscosity: see Table 47.

TABLE 47
Viscosity of a Chitin 30% oil in water suspension

| Shear Rate (Hz) | Viscosity (mPa · s) |
|---|---|
| 0.14 | 286700 |
| 0.28 | 98000 |
| 0.56 | 51670 |
| 1.4 | 20950 |
| 2.24 | 11790 |
| 2.8 | 10500 |

2) Chitosan with Oil

The chitosan suspension was generated by milling chitosan in water with oil in a ratio of either, 1:18:2 (10% oil), 1:17:3 (15% oil), or 1:16:4 (20% oil) at 670 RPM with fifty units of 10 mm ball using the 10+1 Alt method, where it is milled for ten minutes followed by a pause for one minute then milling for ten minutes in the opposite direction for a total of 3 hours.

2.1) Chitosan 10% Oil in Water

Results: Phase separation: No. Formation of agglomerates: homogeneous appearance. Color: off-white/beige. Viscosity: see Table 48.

TABLE 48
Viscosity of a Chitosan 10% oil in water suspension

| Shear Rate (Hz) | Viscosity (mPa · s) |
|---|---|
| 0.14 | 302000 |
| 0.28 | 141400 |
| 0.56 | 64750 |
| 1.4 | 23400 |
| 2.24 | 13120 |
| 2.8 | 9742 |

2.2) Chitosan 15% Oil in Water

Results: Phase separation: no. Formation of agglomerates: homogeneous appearance. Color: light grey. Viscosity: see Table 49.

TABLE 49
Viscosity of a Chitosan oil in water suspension

| Shear Rate (Hz) | Viscosity (mPa · s) |
|---|---|
| 0.14 | 58330 |
| 0.28 | 22170 |
| 0.56 | 9375 |
| 1.4 | 3617 |
| 2.24 | 2917 |
| 2.8 | 2075 |

2.3) Chitosan 20% Oil in Water

Results: Phase separation: small amount ˜1 mL; Formation of agglomerates: homogeneous appearance. Color: light grey. Viscosity: see Table 50.

TABLE 50
Viscosity of a Chitosan 20% oil in water suspension

| Shear Rate (Hz) | Viscosity (mPa · s) |
|---|---|
| 0.14 | 498200 |
| 0.28 | 220500 |
| 0.56 | 92750 |
| 1.4 | 32400 |
| 2.24 | 18640 |
| 2.8 | 13970 |

3) Cellulose with Oil

The CXC cellulose suspension was generated by milling cellulose in water with oil in a ratio of either, 1:18:2 (10% oil), 1:16:4 (20% oil), 1:14:6 (30% oil), 1:12:8 (40% oil) or 1:10:10 (50% oil at 670 RPM with fifty units of 10 mm ball using the 10+1 Alt method, where it is milled for ten minutes followed by a pause for one minute then milling for ten minutes in the opposite direction for a total of 3 hours.

3.1) Cellulose 10% Oil in Water

Results: Phase separation: no. Formation of agglomerates: homogeneous appearance. Color: white. Viscosity: see Table 51.

TABLE 51
Viscosity of a Cellulose 10% oil in water suspension

| Shear Rate (Hz) | Viscosity (mPa · s) |
|---|---|
| 0.14 | 184500 |
| 0.28 | 56500 |
| 0.56 | 26540 |
| 1.4 | 13230 |
| 2.24 | 9521 |
| 2.8 | 8425 |

3.2) Cellulose 20% Oil in Water

Results: Phase separation: no. Formation of agglomerates: homogeneous appearance. Color: white. Viscosity: see Table 52.

TABLE 52
Viscosity of a Cellulose 20% oil in water suspension

| Shear Rate (Hz) | Viscosity (mPa · s) |
|---|---|
| 0.14 | 274200 |
| 0.28 | 133400 |
| 0.56 | 60960 |
| 1.4 | 24370 |
| 2.24 | 16270 |
| 2.8 | 15090 |

3.3) Cellulose 30% Oil in Water

Results: Phase separation: no. Formation of agglomerates: homogeneous appearance. Color: white. Viscosity: see Table 53.

TABLE 53
Viscosity of a Cellulose 30% oil in water suspension

| Shear Rate (Hz) | Viscosity (mPa · s) |
|---|---|
| 0.14 | 390200 |
| 0.28 | 210200 |
| 0.56 | 102300 |
| 1.4 | 39420 |
| 2.24 | 22520 |
| 2.8 | 17470 |

3.4) Cellulose 40% Oil in Water

Results: Phase separation: no. Formation of agglomerates: homogeneous appearance; Color: white. Viscosity: see Table 54.

TABLE 54
Viscosity of a Cellulose 40% oil in water suspension

| Shear Rate (Hz) | Viscosity (mPa · s) |
|---|---|
| 0.14 | 584800 |
| 0.28 | 318800 |
| 0.56 | 168600 |
| 1.4 | 71650 |
| 2.24 | 46510 |
| 2.8 | 36580 |

Conclusions

Oil incorporation into chitin, chitosan and cellulose suspension is possible to significant amounts, above 10%. Chitin was stable up to at least 30% oil; Chitosan was stable up to at least 20% oil, and cellulose was stable up to at least 40% oil. This shows the emulsifying capability of the polymers as Pickering agents.

### Example 25: Ranges of Incorporation of Chitin Chitosan and Cellulose in Suspensions

Tests were carried to help define possible ranges of biopolymer incorporation in suspensions. This was accomplished by starting at a high biopolymer to water ratio follow by an increase in the biopolymer quantity until appearance of a non-homogeneous suspension, i.e., presence of non-suspended particles, or a viscosity that prevents processing via the planetary mill (clumps together with the balls in the jar). Homogeneity and smoothness were further assessed.

1) Chitin

The chitin suspension was generated by milling chitin in water with a chitin to water ratio of either 3:20, 4:20, or 5:20 ratio at 670 RPM with fifty units of 10 mm ball using the 10+1 Alt method, where it is milled for ten minutes followed by a pause for one minute then milling for ten minutes in the opposite direction for a total of 3 hours. The results are presented in Table 55.

TABLE 55
Appearance of various chitin suspensions

| chitin to water ratio | Appearance |
|---|---|
| 3:20 | Homogeneous, non-flowing Smooth |
| 4:20 | Homogeneous, non-flowing, Smooth |
| 5:20 | Homogeneous, non-flowing, Smooth |

2) Chitosan

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The chitosan suspension was generated by milling chitosan in water with a chitosan to water ratio of either 3:20, 4:20, or 5:20 ratio at 670 RPM with fifty units of 10 mm ball using the 10+1 Alt method, where it is milled for ten minutes followed by a pause for one minute then milling for ten minutes in the opposite direction for a total of 3 hours. The results are presented in Table 56 and Table 57.

TABLE 56
Appearance of various Chitosan suspensions

| Chitosan to water ratio | Appearance |
|---|---|
| 3:20 | Homogeneous, flowing, Smooth |
| 4:20 | Homogeneous, non-flowing, Smooth |
| 5:20 | Homogeneous, non-flowing, Smooth |

TABLE 57
Viscosity 3:20 chitosan:water suspension

| Shear Rate (Hz) | Viscosity (mPa · s) |
|---|---|
| 0.14 | 173000 |
| 0.28 | 74670 |
| 0.56 | 29460 |
| 1.4 | 9867 |
| 2.24 | 5406 |
| 2.8 | 4133 |

3) Cellulose

The cellulose suspension was generated by milling cellulose in water with a cellulose to water ratio of either 3:20, or 4:20, 5:20 at 670 RPM with fifty units of 10 mm ball using the 10+1 Alt method, where it is milled for ten minutes followed by a pause for one minute then milling for ten minutes in the opposite direction for a total of 3 hours. The results are presented in Table 58.

TABLE 58
Appearance of various cellulose suspensions

| chitin to water ratio | Appearance |
|---|---|
| 3:20 | Homogeneous, non-flowing Smooth |
| 4:20 | Homogeneous, non-flowing, Smooth |

Conclusions

With all three biopolymers tested—chitin, chitosan and cellulose—suspension occurs with biopolymer to water ratios of at least 3:20, at least 4:20, or at least 5:20.

### Example 26: pH Stability of Chitin Chitosan and Cellulose

In cosmetics, the pH of the ingredients and mixtures can vary. A base material that can accommodate a wide pH range improves applicability.

Accordingly, tests were carried to help define possible ranges of pH. To do so, the pH biopolymer suspensions was altered to extremes, low and high pH, to test the effect on the stability of the suspensions. pH of the suspension mixtures were measured with pH paper. Visual observational results were noted for the following: phase separation, formation of agglomerates, and color change.

1) Chitin (Starting pH: 6-7)

1.1) Chitin Acid Test

The chitin suspension was generated by milling chitin in water with a chitin to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours.

The chitin suspension (6.08 g) and 1M HCl (5.45 g) were combined and vortexed for 20 seconds. Results: Phase separation: none; formation of agglomerates: none; color change: none; final pH: ˜1.

1.2) Chitin Base Test

The chitin suspension (6.15 g) and 1M NaOH (5.09 g) were combined and vortexed for 20 seconds. Results: phase separation: none; formation of agglomerates: none; color change: none; final pH: >12.

2) Chitosan (Starting pH: 7-8)

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours.

The chitosan suspension was generated by milling chitosan in water with a chitosan to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours.

2.1) Chitosan Acid Test

The chitosan suspension (6.03 g) and 1M HCl (5.11 g) were combined and vortexed for 20 seconds. Results: phase separation: none; formation of agglomerates: none; color change: opaque translucent off-white; final pH: ˜1.

2.1) Chitosan Base Test

The chitosan suspension (6.08 g) and 1M NaOH (5.01 g) were combined and vortexed for 20 seconds. Results: phase separation: none; formation of agglomerates: none; color change: none; final pH: >12.

3) Cellulose (Starting pH: 6-7)

The cellulose suspension was generated by milling cellulose in water with a cellulose to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours.

3.1) Cellulose Acid Test

The cellulose suspension, 6.12 g and 1M HCl, and 5.24 g were combined and vortexed for 20 seconds. Results: phase separation: none; formation of agglomerates: none; color change: none; final pH: ˜1.

3.2) Cellulose Base Test

The Cellulose suspension (6.01 g) and 1M NaOH (5.04 g) were combined and vortexed for 20 seconds. Results: phase separation: none; formation of agglomerates: none; color change: none; final pH: >12.

Conclusions

All samples appear stable (i.e., no separation) at a pH range 1 to 12. The Chitosan suspension with acid changed from a solid opaque suspension to a translucent opaque suspension. This is to be expected as chitosan does dissolve in acid, although it did not form a transparent dissolved polymer solution.

### Example 27: Complete Formulations of Chitin Chitosan and Cellulose Suspensions

Complete formulations of the biopolymer suspensions were generated, the formulations including additives for preservation and for emulsifying. The formulation stability was further tested by the addition of mineral oil.

For all the particular examples listed below: 1) Benzoic acid was used as the preservative; 2) Glyceryl stearate and cetyl alcohol were used as an emulsifier; 3) Centrifuge separation test was conducted to test phase separation of water from the biopolymer suspension phase; 4) Viscosity was measured for the final composition.

1) Chitin

1.1) Chitin with an Emulsifier and a Preservative

The chitin suspension was generated by milling chitin in water with a chitin to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate and Benzoic Acid were added to create a chitin to glyceryl stearate to benzoic acid to water ratio of 1:1.25:0.10:20 then milled under the same conditions for 3 hours.

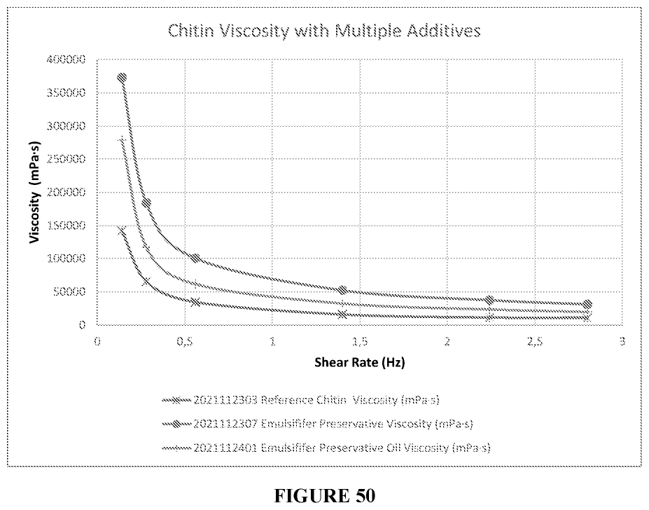

Viscosity of the suspension is shown in FIG. 50. A centrifuge separation test, 10 mins @ 4000 RPM showed some separation, ˜100 μL.

1.2) Chitin with an Emulsifier, a Preservative and Oil

The chitin suspension was generated by milling chitin in water with a chitin to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate and Benzoic Acid were added to create a chitin to glyceryl stearate to benzoic acid to water ratio of 1:1.25:0.10:20 then milled under the same conditions for 3 hours.

Mineral oil was added in the stage yielding a final ratio of chitin to glyceryl stearate to benzoic acid to mineral oil to water ratio of 1:1.25:0.10:0.50:20 then milled under the same conditions for 3 hours.

Viscosity of the suspension is shown in FIG. 50. A centrifuge separation test, 10 mins @ 4000 RPM showed some separation, ˜0.5 mL.

2) Chitosan

2.1) Chitosan with an Emulsifier and a Preservative

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The CXC chitosan suspension was generated by milling chitosan in water with a chitosan to water ratio of 1.30:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate and Benzoic Acid were added to create a chitosan to glyceryl stearate to benzoic acid to water ratio of 1.30:1.25:0.10:20 then milled under the same conditions for 3 hours.

The viscosity was too viscose to be measured with a Brookfield™ viscometer. A centrifuge separation test, 10 mins @ 4000 RPM showed no separation.

2.2) Chitosan with an Emulsifier, a Preservative and Oil

Chitosan was pre-milled dry at 670 RPM with thirty units of 10 mm ball using the 10+1 Alt method, where it is milled for ten minutes followed by a pause for one minute then milling for ten minutes in the opposite direction for a total of 3 hours. The CXC chitosan suspension was generated by milling chitosan in water with a chitosan to water ratio of 1.30:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate and Benzoic Acid were added to create a chitosan to glyceryl stearate to benzoic acid to water ratio of 1.30:1.25:0.10:20 then milled under the same conditions for 3 hours.

Mineral oil was added in the stage yielding a final ratio of chitosan to glyceryl stearate to benzoic acid to mineral oil to water ratio of 1.30:1.25:0.10:0.50:20 then milled under the same conditions for 3 hours.

The viscosity was too viscose to be measured with a Brookfield™ viscometer. A centrifuge separation test, 10 mins @ 4000 RPM: no separation.

3) Cellulose

3.1) Cellulose with Emulsifier and Preservative

The cellulose suspension was generated by milling cellulose in water with a cellulose to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate and Benzoic Acid were added to create a cellulose to glyceryl stearate to benzoic acid to water ratio of 1:1.25:0.10:20 then milled under the same conditions for 3 hours.

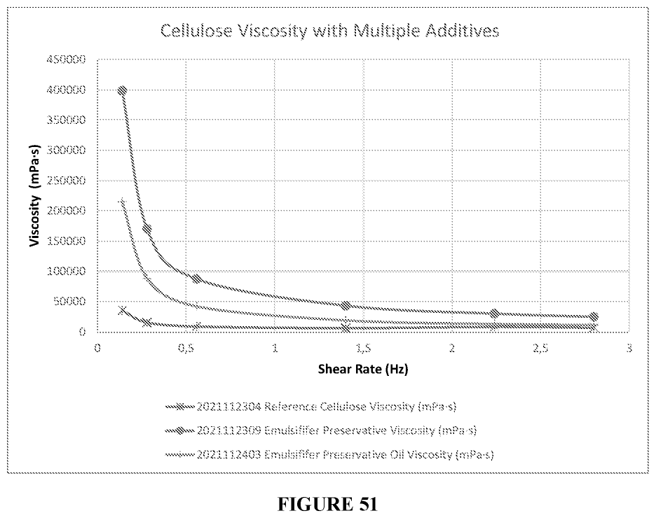

Viscosity of the suspension is shown in FIG. 51. A centrifuge separation test, 10 mins @ 4000 RPM showed some separation, ˜1 mL.

3.2) Cellulose with Emulsifier and Preservative and Oil

The cellulose suspension was generated by milling cellulose in water with a cellulose to water ratio of 1.00:20 at 670 RPM with fifty units of 10 mm ball using the 10+1 Alt method, where it is milled for ten minutes followed by a pause for one minute then milling for ten minutes in the opposite direction for a total of 3 hours. Glyceryl stearate and Benzoic Acid were added to create a cellulose to glyceryl stearate to benzoic acid to water ratio of 1:1.25:0.10:20 then milled under the same conditions for 3 hours.

Mineral oil was added in the stage yielding a final ratio of cellulose to glyceryl stearate to benzoic acid to mineral oil to water ratio of 1:1.25:0.10:0.50:20 then milled under the same conditions for 3 hours.

Viscosity of the suspension is shown in FIG. 51. A centrifuge separation test, 10 mins @ 4000 RPM revealed some separation, ˜3 mL.

Conclusions

The inclusion of emulsifiers and a preservative within the formulations yielded stable chitin, chitosan and cellulose formulations. The further inclusion of oils also produced stable formulations for all three biopolymers. Based on results from the centrifuge separation test, it seems that chitosan formulations exhibited the highest stability, while separation was observed for chitin (to a lower degree) and cellulose (to a more severe degree) formulations.

### Example 28: Large Batch Scale-Up

Chitin, chitosan and cellulose were suspended in a scale up process using the 1.5 L Supermill Plus™, a flow through horizontal mill.

The general milling conditions were 2400 FPM (feet per minute) rotation speed with a pump flow rate of 7.3 GPH (gallons per hour) using 982 mL of 1.4-1.7 mm zirconia beads, where 20 liters of slurry were processed in a 5% solids content (1.05:20).

Chitin, chitosan and cellulose were successfully suspended through the scale up process while milling for 140 mins, producing homogeneous suspensions with the viscosities reported below in Table 59, Table 60 and Table 61.

TABLE 59
Viscosity of Chitin following the scale up process

| Shear Rate (s−1) | Viscosity (mPas) |
|---|---|
| 0.14 | 646000 |
| 0.28 | 341400 |
| 0.56 | 179000 |
| 1.4 | 82050 |
| 2.24 | 55570 |
| 2.8 | 46150 |

TABLE 60
Viscosity of Chitosan following the scale up process

| Shear Rate (s−1) | Viscosity (mPas) |
|---|---|
| 0.14 | 571500 |
| 0.28 | 346000 |
| 0.56 | 185100 |
| 1.4 | 56880 |
| 2.24 | 32510 |
| 2.8 | 25450 |

TABLE 61
Viscosity of Cellulose following the scale up process

| Shear Rate (s−1) | Viscosity (mPas) |
|---|---|
| 0.14 | 614300 |
| 0.28 | 283600 |
| 0.56 | 139000 |
| 1.4 | 58500 |
| 2.24 | 40300 |
| 2.8 | 34580 |

Conclusion

The horizontal media mill can produce biopolymer useful suspensions, showing a successful scale up translation method yielding high viscosity suspensions.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a biopolymer” includes one or more of such biopolymer and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1-3. (canceled)

4. A biopolymer composition comprising a stable homogeneous suspension of an insoluble and/or semi-soluble biopolymer in a polar solvent.

5. (canceled)

6. The biopolymer composition according to claim 4, wherein said insoluble biopolymer is selected from the group consisting of chitin, chitosan, cellulose, hemicellulose, lignin, amylose, actin, fibrin, collagen, silk, fibroin, keratin, wool, alginic acid and mixtures thereof.

7-8. (canceled)

9. The biopolymer composition according to claim 4, wherein the polar solvent is selected from the group consisting of water, ethanol, propanol, methanol, glycerol, isopropanol, acetic acid, and mixtures thereof.

10-14. (canceled)

15. The biopolymer composition according to claim 4, wherein said stable homogeneous suspension comprises biopolymer fibers, and wherein:
said biopolymer fibers have of a width of at least 10 nm, or at least 20 nm, or at least 30 nm, or at least 40 nm, or at least 50 nm, or at least 75 nm, or at least 100 nm, or at least 250 nm, or at least 500 nm, or at least 750 nm, or at least 1 μm, or at least 2 μm, or at least 3 μm, or at least 4 μm, or at least 5 μm, or at least 10 μm or wider; and
said biopolymer fibers have a length of about 50 nm to about 10 μm, or about 100 nm to about 10 μm, or about 500 nm to about 10 μm, or about 750 nm to about 10 μm, or about 800 nm to about 10 μm, or about 900 nm to about 5 μm, or about 1 μm to about 10 μm, or about 1 μm to about 5 μm, or about 1 μm to about 3 μm.

16-20. (canceled)

21. The biopolymer composition according to claim 4, wherein said stable homogeneous suspension comprises at least one of: (i) biopolymer fibers having a globular shape; and (ii) spherical bodies, wherein said spherical bodies comprises an average effective diameter as defined in Table 3, and/or a mean diameter by intensity as defined in Table 3, and/or a mean diameter by volume as defined in Table 3, and/or a mean diameter by number as defined in Table 3.

22-23. (canceled)

24. The biopolymer composition according to claim 4, wherein said stable homogeneous suspension comprises a range of particle sizes, as measured by SEM, as defined in Table 4, or as defined in any one of Tables 30-34.

25. (canceled)

26. The biopolymer composition according to claim 4, comprising at least one of: (i) particles of cellulose having an average size of about 50 nm to about 80 nm; (ii) particles of chitin having an average size of about 45 nm to about 85 nm; (iii) particles of chitosan having an average size of about 75 nm to about 120 nm; (iv) and particles of silk having an average size of about 40 nm to about 165 nm.

27-31. (canceled)

32. The biopolymer composition according to claim 4, wherein said biopolymer composition comprises a viscosity of about 20 mPa*s to about 100 000 mPa*s, or about 20 mPa*s to about 500 mPa*s, or about 1 000 mPa*s to about 40 000 mPa*s, or about 500 mPa*s to about 2 000 mPa*s, or about 1 500 mPa*s to about 30 000 mPa*s, or about 20 000 mPa*s to about 50 000 mPa*s, or about 40 000 mPa*s to about 100 000 mPa*s.

33. The biopolymer composition according to claim 4, wherein said biopolymer composition consists essentially of said biopolymer and water.

34. (canceled)

35. The biopolymer composition according to claim 4, wherein said biopolymer composition is substantially free from any added acid, any added base, any added reactive chemical, and/or any added salt.

36. The biopolymer composition according to claim 4, wherein said biopolymer composition is obtained by a process other than chemical processing.

37. The biopolymer composition according to claim 4, wherein said biopolymer composition is obtained by a process selected from the group consisting of mechanical shearing, sheer thinning, ball milling and colloid milling.

38. (canceled)

39. A cosmetic composition comprising the biopolymer composition defined in claim 4, wherein said cosmetic composition is formulated as a paste, an ointment, a cream, a lotion, a gel or a milk.

40. (canceled)

41. The cosmetic composition of claim 39, wherein said cosmetic composition comprises N-Acetylglucosamine (GlcNAc) and/or oligomers of NAGs.

42. The cosmetic composition of claim 39, wherein said cosmetic composition exhibits anti-aging and/or UV blocking properties.

43. (canceled)

44. A mechanical process for obtaining a biopolymer composition as defined in claim 4, comprising subjecting an insoluble and/or semi-soluble biopolymer to mechanical energy in presence of a polar solvent to obtain a stable homogeneous suspension of said insoluble and/or semi-soluble biopolymer(s).

45. A process for obtaining a biopolymer composition as defined in claim 4, comprising subjecting an insoluble and/or semi-soluble biopolymer to high-shearing conditions in presence of a polar solvent until a change of state is observed and a stable homogeneous suspension of the insoluble and/or semi-soluble biopolymer is obtained.

46. The process of claim 45, wherein the biopolymer and polar solvent are in a biopolymer:solvent weight ratio of about 0.25:20 to about 10:20, or about 0.5:20 to about 3:20.

47. (canceled)

48. The process of claim 45, wherein said high-shearing conditions comprises at least one of mechanical shearing, sheer thinning, planetary ball milling, rolling mill, vibrating ball mill, tumbling stirred ball mill, horizontal media mill, and colloid milling.

49-77. (canceled)

78. The composition of claim 4, further comprising at least one preservative.
